(19) United States
(12) Patent Application Publication
(10) Pub. No.: US 2022/0213542 A1
(43) Pub. Date: Jul. 7, 2022

(54) SEQUENCING BY SYNTHESIS WITH ENERGY TRANSFER DYE PAIRS

(71) Applicants: Jingyue Ju, Englewood Cliffs, NJ (US); Shiv Kumar, Belle Mead, NJ (US); James Russo, New York, NY (US); Xiaoxu Li, New York, NY (US); Steffen Jockusch, New York, NY (US); Minchen Chien, Tenafly, NJ (US); Chuanjuan Tao, Fort Lee, NJ (US)

(72) Inventors: Jingyue Ju, Englewood Cliffs, NJ (US); Shiv Kumar, Belle Mead, NJ (US); James Russo, New York, NY (US); Xiaoxu Li, New York, NY (US); Steffen Jockusch, New York, NY (US); Minchen Chien, Tenafly, NJ (US); Chuanjuan Tao, Fort Lee, NJ (US)

(73) Assignee: THE TRUSTEES OF COLUMBIA UNIVERSITY IN THE CITY OF NEW YORK, New York, NY (US)

(21) Appl. No.: 17/600,593

(22) PCT Filed: Apr. 2, 2020
(86) PCT No.: PCT/US2020/026378

§ 371 Date: Sep. 30, 2021

Related U.S. Application Data
(60) Provisional application No. 62/828,031, filed on Apr. 2, 2019.

(51) Int. Cl. C12Q 1/6869; C12Q 1/6848

(52) U.S. Cl. CPC C12Q 1/6869; C12Q 1/6848

(57) ABSTRACT

The invention provides nucleotide analogues and methods of use thereof for sequencing by synthesis using energy transfer dye pairs.

This application claims priority of U.S. Provisional Application No. 62/828,031, filed Apr. 2, 2019, the contents of which are hereby incorporated by reference.

Throughout this application, various publications and patents are referenced. Full citations for these references may be found at the end of the specification immediately preceding the claims. The disclosures of these publications and patents in their entirety are hereby incorporated by reference into this application to more fully describe the state of the art to which this invention pertains.

### BACKGROUND OF THE INVENTION

DNA sequencing is a fundamental tool in biological and medical research, and is especially important for the paradigm of personalized medicine. Various new DNA sequencing methods have been investigated with the aim of eventually realizing the goal of the $1,000 genome; the dominant method is sequencing by synthesis (SBS), an approach that determines DNA sequences during the polymerase reaction (Hyman 1988; Ronaghi et al. 1998; Ju et al. 2003; Li 2003; Braslavsky et al. 2003; Ruparel et al. 2005; Margulies et al. 2005; Ju et al. 2006; Wu et al. 2007; Guo et al. 2008; Bentley et al. 2008; Harris et al. 2008; Eid et al. 2009; Rothberg et al. 2011).

Presented herein are two novel classes of energy transfer-based sequencing by synthesis approaches.

### SUMMARY

The invention disclosed herein provides a method of sequencing nucleic acid comprising:
a) providing at least one nucleic acid template hybridized to a primer;
b) providing a first nucleic acid polymerase and extending the primer hybridized to said at least one nucleic acid template with the first nucleic acid polymerase and a fluorescently labeled nucleotide analogue if the nucleotide analogue is complementary to a nucleotide residue which is immediately 5′ to the nucleotide residue of the nucleic acid template hybridized to the 3′ terminal nucleotide residue of the primer, wherein the nucleotide analogue is either:
   (i) a fluorescently labeled nucleotide analogue comprising a base and a fluorescent label and a blocking group attached to the base of the nucleotide analogue via a cleavable linker, wherein said blocking group prevents or greatly reduces incorporation of a subsequent nucleotide analogue into the extended primer strand, and wherein said fluorescent label comprises an energy transfer acceptor or donor dye; or
   (ii) a fluorescently labeled reversibly blocked nucleotide analogue comprising a base and a fluorescent label attached to the nucleotide analogue via a cleavable linker and a blocking group at the 3′-OH position, wherein said blocking group prevents incorporation of a subsequent nucleotide analogue into the extended primer strand, and wherein the fluorescent label comprises an energy transfer acceptor or donor dye;
c) removing the first nucleic acid polymerase and providing a second nucleic acid polymerase having an attached fluorescent label, wherein said fluorescent label comprises an energy transfer acceptor or donor dye for the energy transfer acceptor or donor dye attached to the nucleotide analogue in step b;
d) identifying the fluorescence signal due to incorporation of the fluorescently labeled nucleotide analogue onto the primer;
e) optionally extending any unextended primer with a 3′ blocked nucleotide analogue without any base modifications;
f) cleaving the label and the blocking group from any incorporated nucleotide analogue of step b);
g) wherein if no fluorescence signal is detected in step d), iteratively repeating steps b) to f) with a fluorescently labeled nucleotide analogue having a different base until the fluorescently labeled nucleotide analogue is incorporated;
h) optionally extending any unextended primer with a 3′ blocked nucleotide analogue without any base modifications if the optional chase step e) was not carried out;
i) iteratively performing steps b) to h) for each nucleotide residue of the nucleic acid template, thereby determining the sequence of the nucleic acid template.

The invention further provides a method of sequencing nucleic acid comprising:
a) providing at least one nucleic acid template hybridized to a primer;
b) providing a first nucleic acid polymerase and four different labeled nucleotide analogues (A, C, T, G) and extending the primer hybridized to said at least one nucleic acid template with the first nucleic acid polymerase and one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) fluorescently labeled nucleotide analogues comprising a base and a blocking group linked to the base via a cleavable linker and a fluorescent label linked distal to the blocking group via either an uncleavable or a different cleavable linker, wherein said blocking group prevents or greatly reduces incorporation of a subsequent nucleotide analogue into the extended primer strand, wherein each of the different nucleotide analogues (A, C, G, T) have the same fluorescent label and different cleavable linkers, and wherein said fluorescent label comprises an energy transfer acceptor or donor dye; or
   (ii) fluorescently labeled nucleotide analogues comprising a base and a fluorescent label attached to the base via a cleavable linker and a blocking group at the 3′-OH position, wherein said blocking group prevents incorporation of a subsequent nucleotide analogue into the extended primer strand, and wherein each of the different nucleotide analogues (A, C, G, T) have the same fluorescent label and different cleavable linkers, and wherein said fluorescent label comprises an energy transfer acceptor or donor dye;
c) removing the first nucleic acid polymerase and providing a second nucleic acid polymerase having an attached fluorescent label, wherein said fluorescent label comprises an energy transfer acceptor or donor dye for the energy transfer acceptor or donor dye attached to the nucleotide analogue incorporated in step b;
d) optionally extending any unextended primer with a 3′ blocked nucleotide analogue without any base modifications;
e) identifying the fluorescence resonance energy transfer (FRET) signal due to incorporation of the fluorescently labeled nucleotide analogue;
f) contacting the incorporated labeled nucleotide analogues with a cleaving agent that cleaves the cleavable linker to remove the label from one of the four different labeled nucleotide analogues, wherein said cleaving agent does not cleave the cleavable label from the remaining labeled nucleotide analogues;
g) replenishing the second nucleic acid polymerase and identifying any loss of FRET signal due to the cleavage carried out in step f) to partially or completely identify the incorporated nucleotide;
h) iteratively repeating steps f) and g) with a cleaving agent that cleaves the cleavable linker to remove the label from a different labeled nucleotide analogue, wherein said cleaving agent does not cleave the label from the remaining labeled nucleotide analogues;
i) determining the labeled nucleotide analogue incorporated in step b) by comparing the results obtained in the multiple iterations of step g); and
j) cleaving the blocking group and at the same time cleaving any remaining fluorescent labels from the extended primers, and iteratively carrying out steps b to j to obtain the sequence of the nucleic acid template.

The invention further provides a method of sequencing nucleic acid comprising:
a) providing at least one nucleic acid template hybridized to a primer;
b) providing a first nucleic acid polymerase and four different anchor labeled nucleotide analogues (A, C, T, G) and extending the primer hybridized to said at least one nucleic acid template with the first nucleic acid polymerase and one of the anchor labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different anchor labeled nucleotide analogues are either:
   (i) anchor labeled nucleotide analogues each comprising a base, a blocking group linked to the base via a cleavable linker, and an anchor linked to the base via an uncleavable linker distal to the blocking group, wherein said blocking group prevents or greatly reduces incorporation of a subsequent nucleotide analogue into the extended primer strand, wherein each different anchor labeled nucleotide analogue (A, C, G, T) has a different anchor and the same cleavable linker; or
   (ii) anchor labeled nucleotide analogues each comprising a base, an anchor linked to the base via a cleavable linker, and a blocking group at the 3′-OH position, wherein said blocking group prevents incorporation of a subsequent nucleotide analogue into the extended primer strand, and wherein each different anchor labeled nucleotide analogue (A, C, G, T) has different anchor from the remaining anchor labeled nucleotide analogues and the same linker;
c) removing the first nucleic acid polymerase and providing a second nucleic acid polymerase having an attached fluorescent label, wherein said fluorescent label attached to the polymerase is an energy transfer donor or acceptor dye;
d) optionally extending any unextended primer with a 3′ blocked nucleotide analogue without any base modifications;
e) identifying any background fluorescence resonance energy transfer (FRET) signal;
f) labeling any primer extension products with a fluorescently labeled anchor binding molecule specific for one of the four anchors of the nucleotide analogues of step b), wherein the anchor binding molecule comprises a fluorescent label, wherein said fluorescent label is an energy transfer donor or acceptor dye for the energy transfer donor or acceptor dye attached to the second nucleic acid polymerase;
g) optionally replenishing the second nucleic acid polymerase and identifying any fluorescence resonance energy transfer (FRET) signal due to the anchor binding molecule binding to the anchor labeled nucleotide analogue incorporated in step b);
h) iteratively repeating steps f) and g) with a fluorescently labeled anchor binding molecule specific for each of the remaining anchor labeled nucleotide analogues one by one, wherein the same fluorescent dye is attached to all four anchor binding molecules;
i) determining the specific nucleotide analogue incorporated by comparing the results obtained in the multiple iterations of step g);
j) contacting the incorporated with a cleaving agent to cleave the blocking group and the anchor and fluorescent labels from the incorporated nucleotide analogue of step b); and iteratively carrying out steps b) to j) to thereby obtain the sequence of the nucleic acid template.

The invention further provides a method of sequencing nucleic acid comprising:
a) providing at least one nucleic acid template hybridized to a primer;
b) providing a first nucleic acid polymerase and four different anchor labeled nucleotide analogues (A, C, T, G) and extending the primer hybridized to said at least one nucleic acid template with the first nucleic acid polymerase and one of the anchor labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different anchor labeled nucleotide analogues are either:
   (i) anchor labeled nucleotide analogues each comprising a base and a blocking group linked to the base via the same cleavable linker, wherein said blocking group prevents or greatly reduces incorporation of a subsequent nucleotide analogue into the extended primer strand, wherein two of the different anchor labeled nucleotide analogues (A, C, G, T) comprise the same anchor and the remaining two different anchor labeled nucleotide analogues comprise the same anchor, wherein the anchor of two of the anchor labeled nucleotide analogues is attached distal to the blocking group via an uncleavable linker and the anchor of each of two of the anchor labeled nucleotide analogues is attached distal to the blocking group via the same cleavable linker; or
   (ii) anchor labeled nucleotide analogues each comprising a base, an anchor attached to the base via a cleavable linker, and a blocking group at the 3′-OH position, wherein said blocking group prevents incorporation of a subsequent nucleotide analogue into the extended primer strand, wherein two of the different anchor labeled nucleotide analogues (A, C, G, T) comprise the same anchor and the remaining two different anchor labeled nucleotide analogues comprise the same anchor, wherein the cleavable linker of two of the anchor labeled nucleotide analogues is the same, and wherein the cleavable linker of the remaining two anchor labeled nucleotide analogues is the same and different cleavable groups;
c) removing the first nucleic acid polymerase and providing a second nucleic acid polymerase having an attached fluorescent label, wherein said fluorescent label attached to the polymerase is an energy transfer donor or acceptor dye;
d) optionally extending any unextended primer with a 3′ blocked nucleotide analogue without any base modifications;
e) identifying any background fluorescence resonance energy transfer (FRET) signal;
f) labeling any primer extension products with a fluorescently labeled anchor binding molecule specific for one of the anchors of the nucleotide analogues of step b), wherein the anchor binding molecule comprises a fluorescent label, wherein said fluorescent label is an energy transfer donor or acceptor dye for the energy transfer donor or acceptor dye attached to the second nucleic acid polymerase;
g) identifying newly generated FRET signals due to the labeling in step f) to partially identify the incorporated nucleotide analogue of step b);
h) repeating steps e and f with a second fluorescently labeled anchor binding molecule specific for the second anchor;
i) cleaving the dye from the fluorescently labeled nucleotides with a specific cleavable agent that cleaves one of the cleavable linkers but does not cleave any remaining linker;
j) optionally replenishing the second nucleic acid polymerase and identifying loss of FRET signal due to the cleavage carried out in step i);
k) determining the specific nucleotide analogue incorporated by comparing the results obtained in steps g) and j);
l) cleaving the blocking group and at the same time cleaving the remaining anchors and fluorescent labels from any extended primers;
   and iteratively carrying out steps b) to l) to obtain the sequence of the nucleic acid template.

The invention further provides a method of sequencing nucleic acid comprising:
a) providing at least one nucleic acid template hybridized to a primer;
b) providing a first nucleic acid polymerase, four different labeled nucleotide analogues (A, C, T, G) and extending the primer hybridized to said at least one nucleic acid template with the first nucleic acid polymerase and one of the nucleotide analogues if the nucleotide analogue is complementary to a nucleotide residue which is immediately 5′ to the nucleotide residue of the nucleic acid template hybridized to the 3′ terminal nucleotide residue of the primer, wherein the four labeled nucleotide analogues are either:
   (i) two different fluorescently labeled nucleotide analogues that comprise a base and a blocking group linked to the base via a first cleavable linker,
      wherein one of the fluorescently labeled nucleotide analogues comprises a fluorescent label attached distal to the blocking group via a second cleavable linker wherein said fluorescent label is an energy transfer donor or acceptor dye, and
      wherein the remaining fluorescently labeled nucleotide analogue comprises a fluorescent label attached distal to the blocking group via an uncleavable linker wherein said fluorescent label is an energy transfer donor or acceptor dye, and
   two different anchor labeled nucleotide analogues that comprise a base and a blocking group linked to the base via the first cleavable linker,
      wherein one of the anchor labeled nucleotide analogues comprises an anchor attached distal to the blocking group via the second cleavable linker, and
      wherein one the remaining anchor labeled nucleotide analogue comprises the same anchor attached distal to the blocking group via an uncleavable linker;
   (ii) two different fluorescently labeled nucleotide analogues that comprise a base and a blocking group at the 3′-OH position,
      wherein one of the fluorescently labeled nucleotide analogues comprises a fluorescent label attached to the base via a first cleavable linker wherein said fluorescent label is an energy transfer donor or acceptor dye, and
      wherein the remaining fluorescently labeled nucleotide analogue comprises a fluorescent label attached to the base via a second cleavable linker wherein said fluorescent label is an energy transfer donor or acceptor dye, and
   two different anchor labeled nucleotide analogues that comprise a base and a blocking group at the 3′-OH position,
      wherein one of the anchor labeled nucleotide analogues comprises an anchor linked to the base via the first cleavable linker, and
      wherein the remaining anchor labeled nucleotide analogue comprises the same anchor linked to the base via a second cleavable linker;
c) removing the first nucleic acid polymerase and providing a second nucleic acid polymerase having an attached fluorescent label, wherein said fluorescent label attached to the polymerase is an energy transfer donor or acceptor dye for the fluorescent label of the fluorescently labeled nucleotide analogues;
d) optionally extending any unextended primer with a 3′ blocked nucleotide analogue without any base modifications;
e) identifying the fluorescence resonance energy transfer (FRET) signal due to incorporation of any fluorescently labeled nucleotide analogues;
f) labeling anchor attached primer extension products with a fluorescently labeled anchor binding molecule specific for one of the anchors, wherein the fluorescent label is the same as that on the fluorescently labeled nucleotide analogues;
g) optionally replenishing the second nucleic acid polymerase and identifying any newly generated FRET signals to partially identify the incorporated nucleotides due to the labeling carried out in step f);
h) cleaving the dye from the fluorescently labeled nucleotides with a specific cleaving agent that cleaves one of the linkers but does not cleave any remaining linkers;
i) optionally replenishing the second nucleic acid polymerase and identifying any loss of FRET signals due to the cleavage carried out in step g);
j) determining the specific nucleotide analogue incorporated by comparing the results obtained in steps g) and i);
k) cleaving the blocking group and at the same time cleaving any remaining anchors and fluorescent labels from the extended primers;
   and iteratively carrying out steps b) to k) to obtain the sequence of the nucleic acid template.

The invention further provides a method of sequencing nucleic acid comprising:
a) providing at least one nucleic acid template hybridized to a primer and a nucleic acid polymerase;
b) providing a first nucleic acid polymerase and extending the primer hybridized to said at least one nucleic acid template with the first nucleic acid polymerase and a fluorescently labeled nucleotide analogue if the nucleotide analogue is complementary to a nucleotide residue which is immediately 5′ to the nucleotide residue of the nucleic acid template hybridized to the 3′ terminal nucleotide residue of the primer, wherein the nucleotide analogue is either:
   (i) a fluorescently labeled nucleotide analogue comprising a base and a fluorescent label and a blocking group attached to the base of the nucleotide analogue via a cleavable linker, wherein said blocking group prevents or greatly reduces incorporation of a subsequent nucleotide analogue into the extended primer strand, and wherein said fluorescent label comprises an energy transfer acceptor or donor dye, and at the same time or immediately afterward providing four unincorporable nucleotide analogues comprising a different fluorescent dye attached to the nucleotide analogue, wherein the fluorescent dye attached to the unincorporable nucleotide analogues is an energy transfer donor or acceptor dye for the fluorescent dye attached to the fluorescently labeled reversibly blocked nucleotide analogue; or
   (ii) a fluorescently labeled reversibly blocked nucleotide analogue comprising a base and a fluorescent label attached to the nucleotide analogue via a cleavable linker and a blocking group at the 3′-OH position, wherein said blocking group prevents incorporation of a subsequent nucleotide analogue into the extended primer strand, and wherein the fluorescent label comprises an energy transfer acceptor or donor dye and at the same time or immediately afterward providing four unincorporable nucleotide analogues comprising a different fluorescent dye attached to the nucleotide analogue, wherein the fluorescent dye attached to the unincorporable nucleotide analogues is an energy transfer donor or acceptor dye for the fluorescent dye attached to the fluorescently labeled reversibly blocked nucleotide analogue;
c) identifying the fluorescence signal due to incorporation of the fluorescently labeled nucleotide analogue onto the primer;
d) cleaving the dye and the blocking group from any primers extended with the fluorescently labeled nucleotide analogues;
e) optionally extending any unextended primer with a 3′ blocked nucleotide analogue without any base modifications;
f) wherein if no fluorescence signal is detected in step c), iteratively repeating steps b) to e) with a fluorescently labeled nucleotide analogue having a different base until the fluorescently labeled nucleotide analogue is incorporated;
g) repeating steps b) to e) with the second one of the four fluorescently labeled nucleotides described in step b;
h) optionally extending any unextended primer with a 3′ blocked nucleotide analogue without any base modifications if the optional chase step e) was not carried out;
i) iteratively performing steps b) to h) for each nucleotide residue of the nucleic acid template, thereby obtaining the sequence of the nucleic acid template.

The invention also provides a method of sequencing nucleic acid comprising:
a) providing at least one nucleic acid template hybridized to a primer;
b) providing a nucleic acid polymerase and four different labeled nucleotide analogues (A, C, T, G) and extending the primer hybridized to said at least one nucleic acid template with the nucleic acid polymerase and one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) fluorescently labeled nucleotide analogues comprising a base and a blocking group linked to the base via a cleavable linker and a fluorescent label linked distal to the blocking group via either an uncleavable or a different cleavable linker, wherein said blocking group prevents or greatly reduces incorporation of a subsequent nucleotide analogue into the extended primer strand, wherein each of the different nucleotide analogues (A, C, G, T) have the same fluorescent label and different cleavable linkers, and wherein said fluorescent label comprises an energy transfer acceptor or donor dye; or
   (ii) fluorescently labeled nucleotide analogues comprising a base and a fluorescent label attached to the base via a cleavable linker and a blocking group at the 3′-OH position, wherein said blocking group prevents incorporation of a subsequent nucleotide analogue into the extended primer strand, and wherein each of the different nucleotide analogues (A, C, G, T) have the same fluorescent label and different cleavable linkers, and wherein said fluorescent label comprises an energy transfer acceptor or donor dye;
c) at the same time as step b) or immediately afterward, providing four different fluorescently labeled unincorporable nucleotide analogues (A, C, T, G), wherein the fluorescent label is an energy transfer donor or acceptor dye for the energy transfer donor or acceptor dye attached to the fluorescently labeled nucleotides of step b);
d) identifying the fluorescence signal due to incorporation of fluorescently labeled nucleotides or nucleotide analogues;
e) cleaving the dye from the fluorescently labeled nucleotides with a cleaving agent that specifically cleaves one of the linkers but does not cleave any remaining linkers;
f) repeating step c) and identifying any loss of fluorescence due to the cleavage carried out in step e) to partially identify the incorporated nucleotide;
g) iteratively repeating steps e) and f) with cleavable agents that specifically cleave any remaining linkers one-by-one;
h) determining the specific nucleotide analogue incorporated in step b) by comparing the results obtained in multiple iterations of steps f) and i);
i) optionally carrying out a chase step with 3′ blocked nucleotides without any base modifications to extend any remaining primers;
j) cleaving the blocking group and at the same time cleaving any remaining fluorescent labels from the extended primers; and iteratively carrying out steps b) to j) to obtain the sequence of the nucleic acid template.

The invention further provides a method of sequencing nucleic acid comprising:
a) a providing at least one nucleic acid template hybridized to a primer;
b) providing a nucleic acid polymerase and four different anchor labeled nucleotide analogues (A, C, T, G) and extending the primer hybridized to said at least one nucleic acid template with the nucleic acid polymerase and one of the anchor labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different anchor labeled nucleotide analogues are either:
   (i) anchor labeled nucleotide analogues each comprising a base, a blocking group linked to the base via a cleavable linker, and an anchor linked to the base via an uncleavable linker distal to the blocking group, wherein said blocking group prevents or greatly reduces incorporation of a subsequent nucleotide analogue into the extended primer strand, wherein each different anchor labeled nucleotide analogue (A, C, G, T) has a different anchor and the same cleavable linker; or
   (ii) anchor labeled nucleotide analogues each comprising a base, an anchor linked to the base via a cleavable linker, and a blocking group at the 3′-OH position, wherein said blocking group prevents incorporation of a subsequent nucleotide analogue into the extended primer strand, and wherein each different anchor labeled nucleotide analogue (A, C, G, T) has different anchor from the remaining anchor labeled nucleotide analogues and the same linker;
c) at the same time as step b) or immediately afterward, providing four different anchor labeled unincorporable nucleotide analogues, wherein the fluorescent label is an energy transfer donor or acceptor dye for the energy transfer donor or acceptor dye attached to the fluorescently labeled nucleotide analogues of step b);
d) identifying the fluorescence signal due to incorporation of fluorescently labeled nucleotide analogues;
e) labeling anchor attached primer extension products with fluorescently labeled anchor binding molecules, wherein the fluorescent label is the same as that on directly labeled nucleotides or nucleotide analogues and wherein the anchor binding molecule binds to the anchor of a specific nucleotide analogue of step b);
f) repeating step c) and identifying newly generated fluorescence signals to partially identify the incorporated nucleotides due to the labeling carried out in step e);
g) repeating steps e) and f) with the fluorescently labeled anchor binding molecule specific for each of the remaining anchors one by one, wherein the same fluorescent dye is attached to all four anchor binding molecules;
h) determining the specific nucleotide analogue incorporated by comparing the results obtained in multiple iterations of steps f) and g);
i) optionally carrying out a chase step with 3′ blocked nucleotides without any base modifications to extend remaining primers;
j) cleaving the blocking group and at the same time cleaving any remaining anchors and fluorescent labels from the extended primers; and and iteratively carrying out steps b) to j) to obtain the sequence of the nucleic acid template.

The invention further provides a method of sequencing nucleic acid comprising:
a) providing at least one nucleic acid template hybridized to a primer;
b) providing a nucleic acid polymerase and four different anchor labeled nucleotide analogues (A, C, T, G) and extending the primer hybridized to said at least one nucleic acid template with the nucleic acid polymerase and one of the anchor labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different anchor labeled nucleotide analogues are either:
   (i) anchor labeled nucleotide analogues each comprising a base and a blocking group linked to the base via the same cleavable linker, wherein said blocking group prevents or greatly reduces incorporation of a subsequent nucleotide analogue into the extended primer strand, wherein two of the different anchor labeled nucleotide analogues (A, C, G, T) comprise the same anchor and the remaining two different anchor labeled nucleotide analogues comprise the same anchor, wherein the anchor of two of the anchor labeled nucleotide analogues is attached distal to the blocking group via an uncleavable linker and the anchor of each of two of the anchor labeled nucleotide analogues is attached distal to the blocking group via the same cleavable linker; or
   (ii) anchor labeled nucleotide analogues each comprising a base, an anchor attached to the base via a cleavable linker, and a blocking group at the 3′-OH position, wherein said blocking group prevents incorporation of a subsequent nucleotide analogue into the extended primer strand, wherein two of the different anchor labeled nucleotide analogues (A, C, G, T) comprise the same anchor and the remaining two different anchor labeled nucleotide analogues comprise the same anchor, wherein the cleavable linker of two of the anchor labeled nucleotide analogues is the same, and wherein the cleavable linker of the remaining two anchor labeled nucleotide analogues is the same and different cleavable groups;
c) at the same time as step b) or immediately afterward, adding all four fluorescently labeled unincorporable nucleotides or nucleotide analogues, wherein the fluorescent label is an energy transfer donor dye for the energy transfer acceptor dye attached to the fluorescently labeled nucleotides of step b);
d) identifying the fluorescence signal due to incorporation of fluorescently labeled nucleotides or nucleotide analogues;
e) labeling anchor attached primer extension products with a fluorescently labeled anchor binding molecule specific for one of the anchors, wherein the fluorescent label is the same as on all anchor binding molecules;
f) repeating step c) and identifying newly generated fluorescence signals to partially or completely identify the incorporated nucleotides due to the labeling carried out in step d);
g) repeating steps e) and f) with a second fluorescently labeled anchor binding molecule specific for a second anchor;
h) cleaving the dye from the fluorescently labeled nucleotides with a specific cleavable agent that cleaves one of the linkers but does not cleave any remaining linkers;
i) repeating step c) and identifying loss of fluorescence due to the cleavage carried out in step h);
j) determining the specific nucleotide analogue incorporated by comparing the results obtained in steps f) and i);
k) optionally carrying out a chase step with 3′ blocked nucleotides without any base modifications to extend remaining primers;
l) cleaving the blocking group and at the same time cleaving any remaining anchors and fluorescent labels from the extended primers; and

iteratively carrying out steps b) to 1) to obtain the sequence of the nucleic acid template.

A method of sequencing nucleic acid comprising:
a) providing at least one nucleic acid template hybridized to a primer;
b) providing a nucleic acid polymerase, four different labeled nucleotide analogues (A, C, T, G) and extending the primer hybridized to said at least one nucleic acid template with the nucleic acid polymerase and one of the nucleotide analogues if the nucleotide analogue is complementary to a nucleotide residue which is immediately 5′ to the nucleotide residue of the nucleic acid template hybridized to the 3′ terminal nucleotide residue of the primer, wherein the four labeled nucleotide analogues are either:
   (i) two different fluorescently labeled nucleotide analogues that comprise a base and a blocking group linked to the base via a first cleavable linker,
      wherein one of the fluorescently labeled nucleotide analogues comprises a fluorescent label attached distal to the blocking group via a second cleavable linker wherein said fluorescent label is an energy transfer donor or acceptor dye, and
      wherein the remaining fluorescently labeled nucleotide analogue comprises a fluorescent label attached distal to the blocking group via an uncleavable linker wherein said fluorescent label is an energy transfer donor or acceptor dye, and
   two different anchor labeled nucleotide analogues that comprise a base and a blocking group linked to the base via the first cleavable linker,
      wherein one of the anchor labeled nucleotide analogues comprises an anchor attached distal to the blocking group via the second cleavable linker, and
      wherein one the remaining anchor labeled nucleotide analogue comprises the same anchor attached distal to the blocking group via an uncleavable linker; or
   (ii) two different fluorescently labeled nucleotide analogues that comprise a base and a blocking group at the 3′-OH position,
      wherein one of the fluorescently labeled nucleotide analogues comprises a fluorescent label attached to the base via a first cleavable linker wherein said fluorescent label is an energy transfer donor or acceptor dye, and
      wherein the remaining fluorescently labeled nucleotide analogue comprises a fluorescent label attached to the base via a second cleavable linker wherein said fluorescent label is an energy transfer donor or acceptor dye, and
   two different anchor labeled nucleotide analogues that comprise a base and a blocking group at the 3′-OH position,
      wherein one of the anchor labeled nucleotide analogues comprises an anchor linked to the base via the first cleavable linker, and
      wherein the remaining anchor labeled nucleotide analogue comprises the same anchor linked to the base via a second cleavable linker;
c) at the same time as step b or immediately afterward, adding all four fluorescently labeled unincorporable nucleotides or nucleotide analogues, wherein the fluorescent label is an energy transfer donor or acceptor dye for the energy transfer acceptor donor or acceptor dye attached to the fluorescently labeled nucleotides of step b);
d) after a chase step with 3′ blocked nucleotides without any base modifications to extend remaining primers, identifying the fluorescence resonance energy transfer (FRET) signal due to incorporation of any fluorescently labeled nucleotide analogues;
e) labeling anchor attached primer extension products with a fluorescently labeled anchor binding molecule specific for one of the anchors, wherein the fluorescent label is the same as that on the fluorescently labeled nucleotide analogues;
f) repeating step c) and identifying any newly generated FRET signals to partially identify the incorporated nucleotides due to the labeling carried out in step e);
g) cleaving the dye from the fluorescently labeled nucleotides with a specific cleavable agent that cleaves one of the linkers but does not cleave the orthogonal linker;
h) repeating step c) and identifying any loss of FRET signals due to the cleavage carried out in step g) to completely identify the incorporated nucleotide;
i) determining the specific nucleotide analogue incorporated by comparing the results obtained in steps f) and h);
j) cleaving the blocking group and at the same time cleaving any remaining anchors and fluorescent labels from the extended primers; and
   iteratively carrying out steps b) to j) to obtain the sequence of the nucleic acid template.

### BRIEF DESCRIPTION OF THE DRAWINGS

FIGS. 1A-1D: General nucleotide structures for use in some of the sequencing methods disclosed herein. In Schemes P3, P4, P5, P6 and U3, U4, U5, U6, a cleavable linker would be required between the base and the extension blocker of the virtual terminators (or proximal to the blocker), and for four (P5, P6, U5, U6) or three (P3, P4, U3, U4) of the virtual terminators, a second (different) cleavable linker would be needed between the blocker and the dye or anchor (or distal to the blocker). Note that it is not necessary that there be a direct linear link between the blocker and base, or between the blocker and dye/anchor. Thus, in the general structures, the blocker is shown as branching off the linker. All that is essential is that cleavage of the proximal but not the distal linker removes the blocker. In addition to the positions for labeling with dyes and anchors presented herein, these dye or anchor moieties could also be attached to the terminal phosphate.

FIG. 2: Single Molecule or Ensemble Single Color FRET-Based Sequencing by Synthesis with Acceptor Dyes on Nucleotide Reversible Terminators and Donor Dyes on Polymerase. A tag bearing multiple copies of a FRET acceptor dye is attached to the NRTs via a cleavable linker. Template DNA (or RNA) is attached to a solid support (e.g., glass slide or chip). A ternary complex between primer, template, unlabeled polymerase, and NRT is formed, and a labeled NRT complementary to the next base in the template is incorporated at the 3′ end of the primer. A gentle wash step is performed to remove free NRTs and unbound polymerase, under conditions that don't disrupt attachment of the extended primer to the template. A polymerase tagged with the FRET donor dye(s) is added at a sufficient concentration to replace the unlabeled polymerase and an additional gentle wash is carried out to remove the previously bound polymerases. FRET is monitored by exciting the donor dye and detecting emission from the acceptor dye, indicating incorporation. Finally, a cleavage reaction is carried out to remove the dye and blocking group on the NRT, restoring the 3′-OH group, to prepare for the next round of SBS. In each cycle a different one of the four NRTs is added. FRET will only occur with incorporation of the nucleotide complementary to the template strand. For single molecule SBS, the template DNA molecules are spaced just far enough apart so that single molecule reactions will take place at different sites on the surface. For ensemble sequencing, greater separation of template DNA is necessary to permit its clonal amplification (e.g., cluster formation by bridge PCR or emulsion PCR on beads). In alternative single-color schemes, different combinations of cleavable linkers connecting the dye to the NRT or different anchors for attachment of the dye via anchor binding molecules can be carried out (Schemes P1-P10), so that all four NRTs can be added simultaneously. Though acceptor dye-labeled NRTs are illustrated in this figure, the use of virtual terminators for ensemble or single molecule SBS follows essentially the same approach.

FIG. 3A: Examples of Cleavable Linkers for Tag Attachment. These are all compatible with the DTM(SS) group, i.e., cleavage with the reagents listed under appropriate conditions will not reduce the disulfide bond in the same or other nucleotide analogues. In other schemes and figures, are provided examples of the azo, allyl and 2-nitrobenzyl (photocleavable) linkers for use in SBS.

FIG. 3B: Examples of Anchors and Anchor Binding Moieties that Bind Covalently to Each Other for Labeling Reactions. Not shown in this cartoon is the biotin anchor and its binding partner streptavidin which for an extremely strong ionic interaction.

FIG. 4: Nucleotide Reversible Terminators with DTM(SS) Blocked 3′ OH Groups and Energy Transfer Acceptor Dye Cy5 Attached Via DTM(SS) Linker. This set of nucleotides could be used with Schemes P1, P2, U1 and U2.

FIG. 5: Virtual Terminators with Four Alternative Cleavable Linkers (DTM(SS), Azo, Allyl and 2-Nitrobenzyl for Attachment of Energy Transfer Acceptor Dye Cy5. This set of nucleotide analogues can be used with Schemes P3 and U3. Other possible blocking groups for virtual terminators may consist of long polymeric molecules attached to the base.

FIG. 6: Virtual Terminators with Four Alternative Anchors (Biotin, TCO, Tetrazine and DBCO) via Streptavidin, Tetrazine, TCO and Azide (N3) Respectively. This set of nucleotide analogues can be used with Schemes P4 and U4. Other possible blocking groups for virtual terminators may consist of long polymeric molecules attached to the base.

FIG. 7: Virtual Terminators with Two Alternative Cleavable Linkers (DTM(SS) and Azo) and Two Alternative Anchors (Biotin and TCO) for Attachment of Energy Transfer Acceptor Dye Cy5 (Via Streptavidin and Tetrazine Respectively). This set of nucleotide analogues can be used with Schemes P5 and U5. Other possible blocking groups for virtual terminators may consist of long polymeric molecules attached to the base.

FIG. 8: Virtual Terminators with Two Alternative Cleavable Linkers (DTM(SS) and Azo) for Attachment of Either Energy Transfer Acceptor Dye Cy5 via Streptavidin. This set of nucleotide analogues can be used with Schemes P6 and U6. Other possible blocking groups for virtual terminators may consist of long polymeric molecules attached to the base.

FIG. 9: Nucleotide Reversible Terminators with DTM(SS) Blocked 3′-OH Group and Four Alternative Cleavable Linkers (DTM(SS), Azo, Allyl and 2-Nitrobenzyl) for Attachment of the Energy Transfer Acceptor Dye Cy5. This set of nucleotide analogues can be used with Schemes P7 and U7.

FIG. 10: Nucleotide Reversible Terminators with DTM(SS) Blocked 3′-OH Group and Four Alternative Anchors (Biotin, TCO, Tetrazine and DBCO) for Attachment of the Energy Transfer Acceptor Dye Cy5 via Streptavidin, TCO, Tetrazine and Azide (N₃) Respectively. This set of nucleotide analogues can be used with Schemes P8 and U8.

FIG. 11: Nucleotide Reversible Terminators with DTM(SS) Blocked 3′-OH Groups, Two Alternative Linkers (DTM(SS) and Azo) and Two Alternative Anchors (Biotin and TCO) for Attaching the Energy Transfer Acceptor Dye Cy5 (Via Streptavidin and Tetrazine, Respectively). This set of nucleotide analogues can be used with Schemes P9 and U9.

FIG. 12: Nucleotide Reversible Terminators with DTM(SS) Blocked 3′-OH Groups, Two Alternative Linkers (DTM(SS) and Azo) to Attach Cy5 via Streptavidin. This set of nucleotide analogues can be used with Schemes P10 and U10.

FIG. 13: Examples of Unincorporable Nucleotides with Methylene, Amine or Other Group Replacing the Oxygen Atom Between α and β Phosphates or with Rp-isomer of α-Thiophosphate, and with Energy Transfer Donor Dye Cy3 Attached to Either the Base or the Terminal Phosphate. Any of these types of nucleotide analogues can be used in Schemes U1-U10. Though Cy3 is shown as the energy transfer donor dye in this figure, Cy2 or other dyes can also be used with the energy transfer acceptor dye Cy5. Other combinations of donor and acceptor dyes can also be used.

FIG. 14: General Scheme for Synthesis of Unincorporable Nucleotide with Dye Attached to Base. In this case, a synthetic scheme for an α,β-methylene nucleotide triphosphate is presented, but similar schemes exist for the other unincorporable nucleotides shown in FIG. 13, and the number of phosphates can be increased as in FIG. 15.

FIG. 15: General Scheme for Synthesis of Unincorporable Nucleotide with Dye Attached to Terminal Phosphate. In this case, a synthetic scheme for an α,β-methylene nucleotide hexaphosphate is presented, but similar schemes exist for the other unincorporable nucleotides shown in FIG. 13, and the number of phosphates can be decreased (e.g., to a tetraphosphate or pentaphosphate) or increased (e.g., to a heptaphosphate or higher polyphosphate).

FIG. 16: Schematic Showing General (Scheme P1) for SBS with Donor Attached to Polymerase and Acceptor on Nucleotide Reversible Terminators. Each SBS cycle consists of three steps: polymerase reaction to incorporate acceptor-dye labeled nucleotide reversible terminators (3′ blocked or virtual terminators), replacement of unlabeled polymerase with donor dye-attached polymerase to affect energy transfer followed by imaging, and cleavage of the acceptor dye and blocking group to reset for the next cycle. Though extension is shown in each of the three cycles illustrated in this general scheme, only if the correct nucleotide reversible terminator is added, i.e., it is complementary to the nucleotide in the template strand, will it be incorporated into the growing primer strand and FRET observed. The arrows in the figure represent excitation of the donor fluorophore (Cy3), FRET from the donor to the acceptor fluorophore (Cy5), and emission of the acceptor fluorophore. Example structures of FRET-acceptor dye labeled NRTs are presented in FIG. 4 and an example of a FRET-donor dye labeled polymerase is presented in FIG. 2.

FIGS. 17A-17B: Example of Several Cycles of Single Color SBS with 4 Different Templates, FRET Donor Dye on Polymerase, and FRET Acceptor Dye on NRT, with Different NRT Added in Each Cycle (Scheme P2). Multiple cycles are shown. Small cycles (e.g., Steps 1-3, steps 4-6, etc.) for addition of each nucleotide reversible terminator and large cycles (Steps 1-12) indicating addition of all 4 nucleotide reversible terminators are shown in this figure; in all, two full large cycles are presented. The details for three small cycles using a single template are presented in the legend to Scheme P1. Since incorporation will occur only if the appropriate nucleotide is added, i.e., one that is complementary to the next available nucleotide in the template strand, the four different templates shown are extended to different lengths, a total of 4 bases for the top and bottom templates, and a total of 3 bases for the middle two templates. Example structures of FRET-acceptor dye labeled NRTs are presented in FIG. 4.

FIGS. 18A-18B: (Scheme P3) Use of dNTP-Cleavable Linker-Blocker-Dyes (dATP-7-DTM(SS)-Blocker-Allyl-Cy5, dTTP-5-DTM(SS)-Blocker-Cy5, dCTP-5-DTM(SS)-Blocker-Azo-Cy5, dGTP-7-DTM(SS)-Blocker-2-Nitrobenzyl-Cy5) to perform 1-color DNA SBS. Step 1, Addition of DNA polymerase and the four nucleotide analogues (dATP-7-DTM(SS)-Blocker-Allyl-Cy5, dTTP-5-DTM(SS)-Blocker-Cy5, dCTP-5-DTM(SS)-Blocker-Azo-Cy5, dGTP-7-DTM(SS)-Blocker-2-Nitrobenzyl-Cy5) to the immobilized primed DNA template enables the incorporation of the complementary nucleotide analogue to the growing DNA strand to terminate DNA synthesis. Optional Step (not shown), Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the dye labeled dNTPs in step 1. The growing DNA strands are terminated with one of the four dye labeled nucleotide analogues (A, C, G, T) or the same one of the four nucleotide analogues (A, C, G, T) without dye. Step 2, after washing away the unincorporated dye labeled nucleotides, DNA polymerase labeled with Cy3 is added and detection of the unique FRET signal (Cy5 emission signal with excitation of Cy3) confirms incorporation, but does not indicate which nucleotide was incorporated. Step 3, cleavage of Allyl linker by adding Pd(0) to the elongated DNA strands results in removal of Cy5 from incorporated A. Step 4, after washing away the cleaved dyes, Cy3-polymerase is again added and a second round of imaging is performed. Loss of Cy5 signal after Cy3 excitation indicates incorporation of A. Step 5, cleavage of Azo linker by adding sodium dithionite (Na₂S₂O₄) to the elongated DNA strands results in removal of Cy5 from incorporated C. Step 6, after re-addition of Cy3-polymerase and washing away the cleaved dyes, a third round of imaging is performed. Loss of Cy5 signal after Cy3 excitation indicates incorporation of C. Step 7, cleavage of 2-nitrobenzyl linker by treating the elongated DNA strands with 340 nm light results in removal of Cy5 from incorporated G. Step 8, after washing away the cleaved dyes, a fourth round of Cy3-polymerase addition and imaging is performed. Loss of Cy5 signal after Cy3 excitation indicates incorporation of G. Step 9, cleavage of SS linker by adding THP to the elongated DNA strands results in removal of Cy5 from incorporated T and also restores the 3′-OH group on any growing strands extended with a 3′-O—SS(DTM)-dNTP in the optional chase step. Step 10, after washing away the cleaved dyes, an optional final round of Cy3-polymerase addition and imaging is performed. Loss of Cy5 signal after Cy3 excitation confirms incorporation of T. If this optional step is performed, it is necessary to carry out an additional wash to remove the remaining Cy3-polymerase. The DNA products are ready for the next cycle of the DNA sequencing reaction. Structures of nucleotides used in this scheme are presented in FIG. 5. Clusters of acceptor dyes on the nucleotides and/or donor dyes on the polymerase can also be used to amplify the energy transfer signal.

FIGS. 19A-19B: Single Color SBS with Donor Dye on Polymerase, Acceptor Dye on Virtual Terminators, Using 4 Anchors (Scheme P4). Use of dNTP-Cleavable Linker-Blocker-Anchors (dATP-7-DTM(SS)-Blocker-Biotin, dTTP-5-DTM(SS)-Blocker-TCO, dCTP-5-DTM(SS)-Blocker-DBCO, dGTP-7-DTM(SS)-Blocker-Tetrazine) to perform 1-color DNA SBS. Step 1, Addition of DNA polymerase and the four nucleotide analogues (dATP-7-DTM(SS)-Blocker-Biotin, dTTP-5-DTM(SS)-Blocker-TCO, dCTP-5-DTM(SS)-Blocker-DBCO, dCTP-7-DTM(SS)-Blocker-Tetrazine) to the immobilized primed DNA template enables the incorporation of the complementary nucleotide analogue to the growing DNA strand to terminate DNA synthesis. Optional Step (not shown), Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the anchor labeled dNTPs in step 1. The growing DNA strands are terminated with one of the four anchor labeled nucleotide analogues (A, C, G, T) or the same one of the four nucleotide analogues (A, C, G, T) without anchor. Step 2, after washing away the unincorporated dye labeled nucleotides, DNA polymerase labeled with Cy3 is added and imaging (optional) is carried out to reveal any background FRET signal (Cy5 emission signal with excitation of Cy3). Step 3, labeling with Streptavidin-Cy5 to attach the dye to the biotin-containing nucleotide analogues. The dye will bind specifically to the A nucleotide analogue. Step 4, after washing away remaining free label and excess nucleotides, Cy3-polymerase is again added and a second round of imaging is performed. Appearance of Cy5 signal after Cy3 excitation indicates incorporation of A. Step 5, labeling with Tetrazine-Cy5 to attach the dye to the TCO-containing nucleotide analogues. The dye will bind specifically to the T nucleotide analogue. Step 6, after re-addition of Cy3-polymerase and washing away remaining free label and excess nucleotides, a third round of imaging is performed. Appearance of new Cy5 signal after Cy3 excitation indicates incorporation of T. Step 7, labeling with TCO-Cy5 to attach the dye to the Tetrazine-containing nucleotide analogues. The dye will bind specifically to the G nucleotide analogue. Step 8, after washing away free label and excess nucleotides, a fourth round of Cy3-polymerase addition and imaging is performed. Appearance of new Cy5 signal after Cy3 excitation indicates incorporation of G. Step 9, labeling with N₃-Cy5 to attach the dye to the DBCO-containing nucleotide analogues. The dye will bind specifically to the C nucleotide analogue. Step 10, after washing away free label and excess nucleotides, a fifth round of Cy3-polymerase addition and imaging is performed. Appearance of new Cy5 signal after Cy3 excitation indicates incorporation of C. Step 11, cleavage of SS linker by adding THP to the elongated DNA strands restores the 3′-OH group on any growing strands extended with a 3′-O—SS(DTM)-dNTP in the optional chase step. Step 12, after washing away the THP, an optional final round of Cy3-polymerase addition and imaging is performed. If this optional step is performed, it is necessary to carry out an additional wash to remove the remaining Cy3-polymerase. The DNA products are ready for the next cycle of the DNA sequencing reaction. Structures of nucleotides used in this scheme are presented in FIG. 6. Clusters of acceptor dyes on the nucleotides and/or donor dyes on the polymerase can also be used to amplify the energy transfer signal.

FIGS. 20A-20B: Single Color SBS with Donor Dye on Polymerase, Acceptor Dye on Virtual Terminators, Using 2 Anchors and 2 Cleavable Linkers (Scheme P5). Use of dNTP-DTM(SS)-Blocker-Anchors (dATP-7-SS-Blocker-Biotin, dGTP-7-SS-Blocker-TCO), dNTP-DTM(SS)-Blocker-Azo-Anchors (dTTP-5-SS-Blocker-Azo-TCO, dCTP-5-SS-Blocker-Azo-Biotin) and the corresponding Dye Labeled Binding Molecules (Cy5-labeled Streptavidin and Cy5-labeled Tetrazine) to perform 1-color DNA SBS. Step 1, Addition of DNA polymerase and the four nucleotide analogues (dATP-7-SS-Blocker-Biotin, dGTP-7-SS-Blocker-TCO, dTTP-5-SS-Blocker-Azo-TCO, dCTP-5-SS-Blocker-Azo-Biotin) to the immobilized primed DNA template enables the incorporation of the complementary nucleotide analogue to the growing DNA strand to terminate DNA synthesis. Optional Step, Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the Blocker-Anchor dNTPs in step 1. The growing DNA strands are terminated with one of the four Blocker-Anchor labeled nucleotide analogues (A, C, G, T) or the same one of the four nucleotide analogues (A, C, G, T) without dye. Step 2, after washing to remove remaining nucleotides and polymerase, Cy3-labeled DNA polymerase is added and imaging is performed with excitation of the Cy3 to obtain background Cy5 emission. Step 3, labeling with Streptavidin-Cy5 to attach the dye to the biotin-containing nucleotide analogues. The dye will bind specifically to the A and C nucleotide analogues, but not the G and T analogues. Step 4, After washing away remaining free label and excess nucleotides, replacement of the unlabeled polymerase with Cy3-labeled polymerase and imaging results in detection of Cy5 emission signal due to FRET after excitation of Cy3, indicating incorporation of either A or C. Step 5, labeling with Tetrazine-Cy5 to attach the dye to the TCO-containing nucleotide analogues. The dye will bind specifically to the G and T nucleotide analogues, but not the A and C analogues. Step 6, After washing away remaining free label and excess nucleotides and re-addition of DNA polymerase-Cy3, detection of new Cy5 signal after excitation of Cy3 indicates incorporation of either G or T. Next, in Step 7, treatment of the DNA products with sodium dithionite cleaves the azo linker, leading to the removal of acceptor dye on T and C. Step 8, After washing away cleaved dye and re-addition of DNA polymerase-Cy3, imaging for the presence of Cy5 fluorescence after excitation of Cy3 is carried out. In this step, if we have already determined that the incorporated nucleotide could be A or C, loss of Cy5 fluorescence would reveal it to be C, while remaining fluorescence would reveal it to be A. Similarly, for signals previously determined as G or T, loss of Cy5 fluorescence would indicate incorporation of T specifically while remaining fluorescence would indicate incorporation of G. Next, in Step 9, treatment of the DNA products with THP cleaves the SS linker, leading to the removal of the blockers and remaining Cy3 dye, and also restores the 3′-OH group on any growing strands extended with a 3′-O—SS(DTM)-dNTP in the optional chase step. Step 10, after washing away THP, an optional DNA polymerase-Cy3 addition and imaging step will confirm all dyes have been removed, in preparation for the next cycle of sequencing. If this optional step is performed, it is necessary to carry out an additional wash to remove the remaining Cy3-polymerase. Structures of modified nucleotides used in this scheme are shown in FIG. 7. Clusters of acceptor dyes on the nucleotides and/or donor dyes on the polymerase can also be used to amplify the energy transfer signal. FIG. 20B (bottom) illustrates the mode of base calling using digital decoding. The cartoons (circles in rectangles) after each imaging step indicate whether FRET is observed (black circles) or not (white circles). If “1” is assigned to a positive FRET signal and “0” to a negative FRET signal, the unique series of numbers at key imaging steps will indicate the base. Using the labeled nucleotide analogues in this scheme, and the imaging results after the two labeling and first cleavage steps, the series “111” will indicate extension with A (template is T); “011” extension with G (template is C); “001” will indicate extension with T (template is A); “110” will indicate extension with C (template is G). Though these cartoons are not included in Schemes P9, U5 and U9, they would display the same patterns of sequence encoding as for Scheme P5.

FIGS. 21A-21B: Single Color SBS with Donor Dye on Polymerase, Acceptor Dye on Virtual Terminators, Using 1 Anchor and 2 Cleavable Linkers (Scheme P6). Use of dNTP-Cleavable Linker-Blocker-Dyes [dNTP-DTM(SS)-Blocker-Dye (dATP-7-SS-Blocker-Cy5), dNTP-DTM(SS)-Blocker-Azo-Dye (dTTP-5-SS-Blocker-Azo-Cy5)], dNTP-Cleavable Linker-Blocker-Anchors [dNTP-DTM(SS)-Blocker-Anchor (dGTP-7-SS-Blocker-Biotin), dNTP-DTM(SS)-Blocker-Azo-Anchor (dCTP-5-SS-Blocker-Azo-Biotin)] and the corresponding Dye Labeled Binding Molecule (Cy5-labeled Streptavidin) to perform 1-color DNA SBS. Step 1, Addition of DNA polymerase and the four nucleotide analogues (dATP-7-SS-Blocker-Cy5, dTTP-5-SS-Blocker-Azo-Cy5, dGTP-7-SS-Blocker-Biotin, dCTP-5-SS-Blocker-Azo-Biotin) to the immobilized primed DNA template enables the incorporation of the complementary nucleotide analogue to the growing DNA strand to terminate DNA synthesis. Optional Step, Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the Blocker-Dye or Blocker-Anchor dNTPs in step 1. The growing DNA strands are terminated with one of the four Blocker-Dye or Blocker-Anchor labeled nucleotide analogues (A, C, G, T) or the same one of the four nucleotide analogues (A, C, G, T) without dye or anchor. Step 2, after washing to remove remaining nucleotides and polymerase, Cy3-labeled DNA polymerase is added and imaging is performed with excitation of the Cy3 to obtain Cy5 emission due to incorporation of either the A or T nucleotide analogue. Step 3, labeling with Streptavidin-Cy5 to attach the dye to the biotin-containing nucleotide analogues. The dye will bind specifically to the C and G nucleotide analogues. Step 4, After washing away remaining free label and excess nucleotides, replacement of the unlabeled polymerase with Cy3-labeled polymerase and imaging results in detection of Cy5 emission signal due to FRET after excitation of Cy3, indicating incorporation of either C or G. Next, in Step 5, treatment of the DNA products with sodium dithionite cleaves the azo linker, leading to the removal of acceptor dye on T and C. Step 6, After washing away cleaved dye and re-addition of DNA polymerase-Cy3, imaging for the presence of Cy5 fluorescence after excitation of Cy3 is carried out. In this step, if it has already determined that the incorporated nucleotide could be A or T, loss of Cy5 fluorescence would reveal it to be T, while remaining fluorescence would reveal it to be A. Similarly, for signals previously determined as C or G, loss of Cy5 fluorescence would indicate incorporation of C specifically while remaining fluorescence would indicate incorporation of G. Next, in Step 7, treatment of the DNA products with THP cleaves the SS linker, leading to the removal of the blockers and remaining Cy3 dye, and also restores the 3′-OH group on any growing strands extended with a 3′-O—SS(DTM)-dNTP in the optional chase step. Step 8, after washing away THP, an optional DNA polymerase-Cy3 addition and imaging step will confirm all dyes have been removed, in preparation for the next cycle of sequencing. If this optional step is performed, it is necessary to carry out an additional wash to remove the remaining Cy3-polymerase. Structures of modified nucleotides used in this scheme are shown in FIG. 8. Clusters of acceptor dyes on the nucleotides and/or donor dyes on the polymerase can also be used to amplify the energy transfer signal. In this figure, we also illustrate the mode of base calling using digital decoding. The cartoons (circles in rectangles) after each imaging step indicate whether FRET is observed (black circles) or not (white circles). If “1” is assigned to a positive FRET signal and “0” to a negative FRET signal, the unique series of numbers at key imaging steps will indicate the base. Using the labeled nucleotide analogues in this scheme, and the imaging results after the extension, labeling and first cleavage steps, the series “111” will indicate extension with A (template is T); “011” extension with G (template is C); “110” will indicate extension with T (template is A); “010” will indicate extension with C (template is G). Though these cartoons are not included in Schemes P10, U6 and U10, they would display identical patterns of sequence encoding as for Scheme P6.

FIGS. 22A-22B: Single Color SBS with Donor Dye on Polymerase, Acceptor Dye on DTM(SS)-NRTs, Using 4 Cleavable Linkers (Scheme P7). Use of 3′-DTM(SS)-dNTP-Cleavable Linker-Dyes (3′-SS-dATP-7-Allyl-Cy5, 3′-SS-dTTP-5-SS-Cy5, 3′-SS-dCTP-5-Azo-Cy5, 3′-SS-dGTP-7-(2-Nitrobenzyl)-Cy5) to perform 1-color DNA SBS. Step 1, Addition of DNA polymerase and the four nucleotide analogues (3′-SS-dATP-7-Allyl-Cy5, 3′-SS-dTTP-5-SS-Cy5, 3′-SS-dCTP-5-Azo-Cy5, 3′-SS-dGTP-7-(2-Nitrobenzyl)-Cy5) to the immobilized primed DNA template enables the incorporation of the complementary nucleotide analogue to the growing DNA strand to terminate DNA synthesis. Optional Step (not shown), Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the dye labeled dNTPs in step 1. The growing DNA strands are terminated with one of the four dye labeled nucleotide analogues (A, C, G, T) or the same one of the four nucleotide analogues (A, C, G, T) without dye. Step 2, after washing away the unincorporated dye labeled nucleotides, DNA polymerase labeled with Cy3 is added and detection of the unique FRET signal (Cy5 emission signal with excitation of Cy3) confirms incorporation, but does not indicate which nucleotide was incorporated. Step 3, cleavage of Allyl linker by adding Pd(0) to the elongated DNA strands results in removal of Cy5 from incorporated A. Step 4, after washing away the cleaved dyes, Cy3-polymerase is again added and a second round of imaging is performed. Loss of Cy5 signal indicates incorporation of A. Step 5, cleavage of Azo linker by adding sodium dithionite (Na₂S₂O₄) to the elongated DNA strands results in removal of Cy5 from incorporated C. Step 6, after re-addition of Cy3-polymerase and washing away the cleaved dyes, a third round of imaging is performed. Loss of Cy5 signal indicates incorporation of C. Step 7, cleavage of 2-nitrobenzyl linker by treating the elongated DNA strands with 340 nm light results in removal of Cy5 from incorporated G. Step 8, after washing away the cleaved dyes, a fourth round of Cy3-polymerase addition and imaging is performed. Loss of Cy5 signal indicates incorporation of G. Step 9, cleavage of SS linker by adding THP to the elongated DNA strands results in removal of Cy5 from incorporated T and also restores the 3′-OH group on all these nucleotide analogues as well as on all the growing strands extended with a 3′-O—SS(DTM)-dNTP in the optional chase step. Step 10, after washing away the cleaved dyes, an optional final round of Cy3-polymerase addition and imaging is performed. Loss of Cy5 signal confirms incorporation of T. If this optional step is performed, it is necessary to carry out an additional wash to remove the remaining Cy3-polymerase. The DNA products are ready for the next cycle of the DNA sequencing reaction. Structures of nucleotides used in this scheme are presented in FIG. 9. Clusters of acceptor dyes on the nucleotides and/or donor dyes on the polymerase can also be used to amplify the energy transfer signal.

FIGS. 23A-23B: Single Color SBS with Donor Dye on Polymerase, Acceptor Dye on DTM(SS)-NRTs, Using 4 Anchors (Scheme P8). Use of 3′-DTM(SS)-dNTP-Cleavable Linker-Dyes (3′-SS-dATP-7-SS-Biotin, 3′-SS-dTTP-5-SS-Tetrazine, 3′-SS-dCTP-5-SS-TCO, 3′-SS-dGTP-7-SS-DBCO) to perform 1-color DNA SBS. Step 1, Addition of DNA polymerase and the four nucleotide analogues (3′-SS-dATP-7-SS-Biotin, 3′-SS-dTTP-5-SS-Tetrazine, 3′-SS-dCTP-5-SS-TCO, 3′-SS-dGTP-7-SS-DBCO) to the immobilized primed DNA template enables the incorporation of the complementary nucleotide analogue to the growing DNA strand to terminate DNA synthesis. Optional Step (not shown), Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the anchor labeled dNTPs in step 1. The growing DNA strands are terminated with one of the four anchor labeled nucleotide analogues (A, C, G, T) or the same one of the four nucleotide analogues (A, C, G, T) without anchor. Step 2, after washing away the unincorporated dye labeled nucleotides, DNA polymerase labeled with Cy3 is added and imaging is carried out to reveal background FRET signal (Cy5 emission signal with excitation of Cy3). Step 3, labeling with Streptavidin-Cy5 to attach the dye to the biotin-containing nucleotide analogues. The dye will bind specifically to the A nucleotide analogue. Step 4, after washing away remaining free label and excess nucleotides, Cy3-polymerase is again added and a second round of imaging is performed. Appearance of Cy5 signal after Cy3 excitation indicates incorporation of A. Step 5, labeling with Tetrazine-Cy5 to attach the dye to the TCO-containing nucleotide analogues. The dye will bind specifically to the C nucleotide analogue. Step 6, after re-addition of Cy3-polymerase and washing away remaining free label and excess nucleotides, a third round of imaging is performed. Appearance of new Cy5 signal after Cy3 excitation indicates incorporation of C. Step 7, labeling with TCO-Cy5 to attach the dye to the Tetrazine-containing nucleotide analogues. The dye will bind specifically to the T nucleotide analogue. Step 8, after washing away free label and excess nucleotides, a fourth round of Cy3-polymerase addition and imaging is performed. Appearance of new Cy5 signal after Cy3 excitation indicates incorporation of T. Step 9, labeling with N₃-Cy5 to attach the dye to the DBCO-containing nucleotide analogues. The dye will bind specifically to the G nucleotide analogue. Step 10, after washing away free label and excess nucleotides, a fifth round of Cy3-polymerase addition and imaging is performed. Appearance of new Cy5 signal after Cy3 excitation indicates incorporation of G. Step 11, cleavage of SS linker by adding THP to the elongated DNA strands restores the 3′-OH group on these nucleotide analogues as well as on any growing strands extended with a 3′-O—SS(DTM)-dNTP in the optional chase step. Step 12, after washing away the cleaved dyes, an optional final round of Cy3-polymerase addition and imaging is performed. If this optional step is performed, it is necessary to carry out an additional wash to remove the remaining Cy3-polymerase. The DNA products are ready for the next cycle of the DNA sequencing reaction. Structures of nucleotides used in this scheme are presented in FIG. 10. Clusters of acceptor dyes on the nucleotides and/or donor dyes on the polymerase can also be used to amplify the energy transfer signal.

FIGS. 24A-24B: Single Color SBS with Donor Dye on Polymerase, Acceptor Dye on DTM(SS)-NRTs, Using 2 Anchors and 2 Cleavable Linkers (Scheme P9). Use of 3′-DTM(SS)-dNTP-DTM(SS)-Anchor (3′-SS-dATP-7-SS-Biotin, 3′-SS-dGTP-7-SS-TCO), DTM(SS)-dNTP-Azo-Anchors (3′-SS-dTTP-5-Azo-TCO, 3′-SS-dCTP-5-Azo-Biotin) and the corresponding Dye Labeled Binding Molecules (Cy5-labeled Streptavidin and Cy5-labeled Tetrazine) to perform 1-color DNA SBS. Step 1, Addition of DNA polymerase and the four nucleotide analogues (3′-SS-dATP-7-SS-Biotin, 3′-SS-dGTP-7-SS-TCO, 3′-SS-dTTP-5-Azo-TCO, 3′-SS-dCTP-5-Azo-Biotin) to the immobilized primed DNA template enables the incorporation of the complementary nucleotide analogue to the growing DNA strand to terminate DNA synthesis. Optional Step, Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the Anchor labeled NRTs in step 1. The growing DNA strands are terminated with one of the four Anchor labeled nucleotide analogues (A, C, G, T) or the same one of the four nucleotide analogues (A, C, G, T) without anchor. Step 2, after washing to remove remaining nucleotides and polymerase, Cy3-labeled DNA polymerase will be added and imaging will be performed with excitation of the Cy3 to obtain background Cy5 emission. Step 3, labeling with Streptavidin-Cy5 to attach the dye to the biotin-containing nucleotide analogues. The dye will bind specifically to the A and C nucleotide analogues, but not the G and T analogues. Step 4, After washing away remaining free label and excess nucleotides, replacement of the unlabeled polymerase with Cy3-labeled polymerase and imaging results in detection of Cy5 emission signal due to FRET after excitation of Cy3, indicating incorporation of either A or C. Step 5, labeling with Tetrazine-Cy5 to attach the dye to the TCO-containing nucleotide analogues. The dye will bind specifically to the G and T nucleotide analogues, but not the A and C analogues. Step 6, After washing away remaining free label and excess nucleotides and re-addition of DNA polymerase-Cy3, detection of new Cy5 signal after excitation of Cy3 indicates incorporation of either G or T. Next, in Step 7, treatment of the DNA products with sodium dithionite cleaves the azo linker, leading to the removal of acceptor dye on T and C. Step 8, After washing away cleaved dye and re-addition of DNA polymerase-Cy3, imaging for the presence of Cy5 fluorescence after excitation of Cy3 is carried out. In this step, if it has already been determined that the incorporated nucleotide could be A or C, loss of Cy5 fluorescence would reveal it to be C, while remaining fluorescence would reveal it to be A. Similarly, for signals previously determined as G or T, loss of Cy5 fluorescence would indicate incorporation of T specifically while remaining fluorescence would indicate incorporation of G. Next, in Step 9, treatment of the DNA products with THP cleaves the 3′ blocking group and the SS linkers, restoring the 3′-OH and removing any remaining Cy5, and also restores the 3′-OH group on any growing strands extended with a 3′-O—SS(DTM)-dNTP in the optional chase step. Step 10, after washing away THP, an optional DNA polymerase-Cy3 addition and imaging step will confirm all dyes have been removed, in preparation for the next cycle of sequencing. If this optional step is performed, it is necessary to carry out an additional wash to remove the remaining Cy3-polymerase. Structures of modified nucleotides used in this scheme are shown in FIG. 11. Clusters of acceptor dyes on the nucleotides and/or donor dyes on the polymerase can also be used to amplify the energy transfer signal.

FIGS. 25A-25B: Single Color SBS with Donor Dye on Polymerase, Acceptor Dye on DTM(SS)-NRTs, Using 1 Anchor and 2 Cleavable Linkers (Scheme PIG). Use of 3′-DTM(SS)-dNTP-Cleavable Linker-Dyes [3′-DTM(SS)-dNTP—SS-Dye (3′-DTM(SS)-dATP-7-SS-Cy5), 3′-DTM(SS)-dNTP-Azo-Dye (3′-DTM(SS)-dTTP-5-Azo-Cy5)], 3′-DTM(SS)-dNTP-Cleavable Linker-Anchors [3′-DTM(SS)-dNTP-SS-Anchor (3′-DTM(SS)-dGTP-7-SS-Biotin), 3′-DTM(SS)-dNTP-Azo-Anchor (3′-DTM(SS)-dCTP-5-Azo-Biotin)] and the corresponding Dye Labeled Binding Molecule (Cy5-labeled Streptavidin) to perform 1-color DNA SBS. Step 1, Addition of DNA polymerase and the four nucleotide analogues (3′-DTM(SS)-dATP-7-SS-Cy5, 3′-DTM(SS)-dTTP-5-Azo-Cy5, 3′-DTM(SS)-dGTP-7-SS-Biotin, 3′-DTM(SS)-dCTP-5-Azo-Biotin) to the immobilized primed DNA template enables the incorporation of the complementary nucleotide analogue to the growing DNA strand to terminate DNA synthesis. Optional Step, Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the Blocker-Dye or Blocker-Anchor dNTPs in step 1. The growing DNA strands are terminated with one of the four Blocker-Dye or Blocker-Anchor labeled nucleotide analogues (A, C, G, T) or the same one of the four nucleotide analogues (A, C, G, T) without dye or anchor. Step 2, after washing to remove remaining nucleotides and polymerase, Cy3-labeled DNA polymerase is added and imaging is performed with excitation of the Cy3 to obtain Cy5 emission due to incorporation of either the A or T nucleotide analogue. Step 3, labeling with Streptavidin-Cy5 to attach the dye to the biotin-containing nucleotide analogues. The dye will bind specifically to the C and G nucleotide analogues. Step 4, After washing away remaining free label and excess nucleotides, replacement of the unlabeled polymerase with Cy3-labeled polymerase and imaging results in detection of Cy5 emission signal due to FRET after excitation of Cy3, indicating incorporation of either C or G. Next, in Step 5, treatment of the DNA products with sodium dithionite cleaves the azo linker, leading to the removal of acceptor dye on T and C. Step 6, After washing away cleaved dye and re-addition of DNA polymerase-Cy3, imaging for the presence of Cy5 fluorescence after excitation of Cy3 is carried out. In this step, if it has already been determined that the incorporated nucleotide could be A or T, loss of Cy5 fluorescence would reveal it to be T, while remaining fluorescence would reveal it to be A. Similarly, for signals previously determined as C or G, loss of Cy5 fluorescence would indicate incorporation of C specifically while remaining fluorescence would indicate incorporation of G. Next, in Step 7, treatment of the DNA products with THP cleaves the 3′ blocking group and the SS linkers, restoring the 3′-OH and removing any remaining Cy5, and also restores the 3′-OH group on any growing strands extended with a 3′-O—SS(DTM)-dNTP in the optional chase step. Step 8, after washing away THP, an optional DNA polymerase-Cy3 addition and imaging step will confirm all dyes have been removed, in preparation for the next cycle of sequencing. If this optional step is performed, it is necessary to carry out an additional wash to remove the remaining Cy3-polymerase. Structures of modified nucleotides used in this scheme are shown in FIG. 12. Clusters of acceptor dyes on the nucleotides and/or donor dyes on the polymerase can also be used to amplify the energy transfer signal.

FIG. 26: General Scheme for SBS with Acceptor on NRTs and Donor Attached to Adjacent Unincorporable Nucleotides (Scheme U1). Each SBS cycle consists of two steps: polymerase reaction to incorporate acceptor-dye labeled nucleotide reversible terminators (3′ blocked or virtual terminators) and bind unincorporable nucleotides (shown in parentheses and unconnected to prior base) followed by imaging, and cleavage of the acceptor dye and blocking group to reset for the next cycle. Though extension is shown in each of the three cycles illustrated in this general scheme, only if the correct nucleotide reversible terminator is added, i.e., it is complementary to the nucleotide in the template strand, will it be incorporated into the growing primer strand and FRET observed. The arrows in the figure represent excitation of the donor fluorophore (Cy3), FRET from the donor to the acceptor fluorophore (Cy5), and emission of the acceptor fluorophore. The bell pepper-shaped closed curves represent polymerase molecules bound to the template and primer in the ternary complex. Example structures of FRET-acceptor dye labeled NRTs are presented in FIG. 4 and examples of FRET-donor dye labeled unincorporable nucleotides are presented in FIG. 13.

FIGS. 27A-27B: Example of Several Cycles of Single Color SBS with 4 Different Templates, FRET Acceptor Dye on Incoming NRT, and FRET Donor Dye on Unincorporable Nucleotide at Next Position, with Different NRT Added in Each Cycle (Scheme U2). Multiple cycles are shown. Small cycles (e.g., Steps 1-3, steps 4-6, etc.) for addition of each nucleotide reversible terminator and large cycles (Steps 1-12) indicating addition of all 4 nucleotide reversible terminators are shown in this figure; in all, two full large cycles are presented. The details for three small cycles for a single template are presented in the legend to Scheme U1. Since incorporation will occur only if the appropriate nucleotide is added that is complementary to the next available nucleotide in the template strand, the four different templates shown are extended to different lengths, a total of 4 bases for the top and bottom templates, and a total of 3 bases for the middle two templates. Example structures of FRET-acceptor dye labeled NRTs are presented in FIG. 4 and examples of FRET-donor dye labeled unincorporable nucleotides are presented in FIG. 13.

FIGS. 28A-28B: Single Color SBS with Acceptor Dye on Incoming Virtual Terminator, Donor Dye on Unincorporable Nucleotides Binding Transiently at Next Position, Using 4 Cleavable Linkers (Scheme U3). Use of dNTP-Cleavable Linker-Blocker-Dyes (dATP-7-DTM(SS)-Blocker-Allyl-Cy5, dTTP-5-DTM(SS)-Blocker-Cy5, dCTP-5-DTM(SS)-Blocker-Azo-Cy5, dGTP-7-DTM(SS)-Blocker-2-Nitrobenzyl-Cy5) and dNPPCP-Dyes (dAPPCP-Cy3, dCPPCP-Cy3, dGPPCP-Cy3 and dTPPCP-Cy3) to perform 1-color DNA SBS. Step 1, Addition of DNA polymerase and the four nucleotide analogues (dATP-7-DTM(SS)-Blocker-Allyl-Cy5, dTTP-5-DTM(SS)-Blocker-Cy5, dCTP-5-DTM(SS)-Blocker-Azo-Cy5, dGTP-7-DTM(SS)-Blocker-2-Nitrobenzyl-Cy5) to the immobilized primed DNA template enables the incorporation of the complementary nucleotide analogue to the growing DNA strand to terminate DNA synthesis. Optional Step (not shown), Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the dye labeled dNTPs in step 1. The growing DNA strands are terminated with one of the four dye labeled nucleotide analogues (A, C, G, T) or the same one of the four nucleotide analogues (A, C, G, T) without dye. Step 2, after washing away the unincorporated dye labeled nucleotides, DNA polymerase and the four dNPPCP-Cy3 unincorporable nucleotides are added and detection of the unique FRET signal (Cy5 emission signal with excitation of Cy3) confirms incorporation, but does not indicate which nucleotide was incorporated. Step 3, cleavage of Allyl linker by adding Pd(0) to the elongated DNA strands results in removal of Cy5 from incorporated A. Step 4, after washing away the cleaved dyes, DNA polymerase and the dNPPCP-Cy3 nucleotides are again added and a second round of imaging is performed. Loss of Cy5 signal after Cy3 excitation indicates incorporation of A. Step 5, cleavage of Azo linker by adding sodium dithionite (Na₂S₂O₄) to the elongated DNA strands results in removal of Cy5 from incorporated C. Step 6, after re-addition of DNA polymerase and the dNPPCP-Cy3 nucleotides and washing away the cleaved dyes, a third round of imaging is performed. Loss of Cy5 signal after Cy3 excitation indicates incorporation of C. Step 7, cleavage of 2-nitrobenzyl linker by treating the elongated DNA strands with 340 nm light results in removal of Cy5 from incorporated G. Step 8, after washing away the cleaved dyes, a fourth round of DNA polymerase and dNPPCP-Cy3 nucleotide addition and imaging is performed. Loss of Cy5 signal after Cy3 excitation indicates incorporation of G. Step 9, cleavage of SS linker by adding THP to the elongated DNA strands results in removal of Cy5 from incorporated T and also restores the 3′-OH group on any growing strands extended with a 3′-O—SS(DTM)-dNTP in the optional chase step. Step 10, after washing away the cleaved dyes, an optional final round of DNA polymerase and dNPPCP-Cy3 nucleotide addition and imaging is performed. Loss of Cy5 signal after Cy3 excitation confirms incorporation of T. If this optional step is performed, it is necessary to carry out an additional wash to remove any remaining dNPPCP-Cy3. The DNA products are ready for the next cycle of the DNA sequencing reaction. Example structures of FRET-acceptor dye labeled NRTs are presented in FIG. 5 and examples of FRET-donor dye labeled unincorporable nucleotides are presented in FIG. 13. Clusters of acceptor dyes on the nucleotides and/or donor dyes on the polymerase can also be used to amplify the energy transfer signal.

FIGS. 29A-29B: Single Color SBS with Acceptor Dye on Incoming Virtual Terminator, Donor Dye on Unincorporable Nucleotides Binding Transiently at Next Position, Using 4 Anchors (Scheme U4). Use of dNTP-Cleavable Linker-Blocker-Anchors (dATP-7-DTM(SS)-Blocker-Biotin, dTTP-5-DTM(SS)-Blocker-TCO, dCTP-5-DTM(SS)-Blocker-DBCO, dGTP-7-DTM(SS)-Blocker-Tetrazine), the corresponding Cy5-labeled Anchor Binding Molecules (Streptavidin-Cy5, Tetrazine-Cy5, N₃—Cy5 and TCO-Cy5), and Cy3-labeled dNPPCP nucleotide analogues (dAPPCP-Cy3, dCPPCP-Cy3, dGPPCP-Cy3 and dTPPCP-Cy3) to perform 1-color DNA SBS. Step 1, Addition of DNA polymerase and the four nucleotide analogues (dATP-7-DTM(SS)-Blocker-Biotin, dTTP-5-DTM(SS)-Blocker-TCO, dCTP-5-DTM(SS)-Blocker-DBCO, dGTP-7-DTM(SS)-Blocker-Tetrazine) to the immobilized primed DNA template enables the incorporation of the complementary nucleotide analogue to the growing DNA strand to terminate DNA synthesis. Optional Step (not shown), Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the anchor labeled dNTPs in step 1. The growing DNA strands are terminated with one of the four anchor labeled nucleotide analogues (A, C, G, T) or the same one of the four nucleotide analogues (A, C, G, T) without anchor. Step 2, after washing away the unincorporated anchor-labeled nucleotide analogues, addition of DNA polymerase and the four dNPPCP-Cy3 unincorporable nucleotides and imaging (optional) is carried out to reveal any background FRET signal (Cy5 emission signal with excitation of Cy3). Step 3, labeling with Streptavidin-Cy5 to attach the dye to the biotin-containing nucleotide analogues. The dye will bind specifically to the A nucleotide analogue. Step 4, after washing away remaining free label and excess nucleotides, DNA polymerase and the dNPPCP-Cy3 nucleotides are again added and a second round of imaging is performed. Appearance of Cy5 signal after Cy3 excitation indicates incorporation of A. Step 5, labeling with Tetrazine-Cy5 to attach the dye to the TCO-containing nucleotide analogues. The dye will bind specifically to the T nucleotide analogue. Step 6, after washing away remaining free label and excess nucleotides, DNA polymerase and the dNPPCP-Cy3 nucleotides are again added and a third round of imaging is performed. Appearance of new Cy5 signal after Cy3 excitation indicates incorporation of T. Step 7, labeling with TCO-Cy5 to attach the dye to the Tetrazine-containing nucleotide analogues. The dye will bind specifically to the G nucleotide analogue. Step 8, after washing away free label and excess nucleotides, DNA polymerase and the dNPPCP-Cy3 nucleotides are again added and a fourth round of imaging is performed. Appearance of new Cy5 signal after Cy3 excitation indicates incorporation of G. Step 9, labeling with N₃-Cy5 to attach the dye to the DBCO-containing nucleotide analogues. The dye will bind specifically to the C nucleotide analogue. Step 10, after washing away free label and excess nucleotides, DNA polymerase and the dNPPCP-Cy3 nucleotides are again added and a fifth round of imaging is performed. Appearance of new Cy5 signal after Cy3 excitation indicates incorporation of C. Step 11, cleavage of SS linker by adding THP to the elongated DNA strands restores the 3′-OH group on any growing strands extended with a 3′-O—SS(DTM)-dNTP in the optional chase step. Step 12, after washing away the THP, an optional final round of polymerase and dNPPCP-Cy3 nucleotide addition is carried out and imaging is performed. If this optional step is performed, it is necessary to carry out an additional wash to remove the remaining dNPPCP-Cy3 nucleotides and polymerase. The DNA products are ready for the next cycle of the DNA sequencing reaction. Structures of nucleotides used in this scheme are presented in FIG. 6 and examples of FRET-donor dye labeled unincorporable nucleotides are presented in FIG. 13. Clusters of acceptor dyes on the nucleotides and/or donor dyes on the polymerase can also be used to amplify the energy transfer signal.

FIGS. 30A-30B: Single Color SBS with Acceptor Dye on Incoming Virtual Terminator, Donor Dye on Unincorporable Nucleotide Binding Transiently at Next Position, Using 2 Anchors and 2 Cleavable Linkers (Scheme U5). Use of dNTP-DTM(SS)-Blocker-Anchor (dATP-7-SS-Blocker-Biotin, dGTP-7-SS-Blocker-TCO), dNTP-DTM(SS)-Blocker-Azo-Anchors (dTTP-5-SS-Blocker-Azo-TCO, dCTP-5-DTM(SS)-Blocker-Azo-Biotin) the corresponding Dye Labeled Binding Molecules (Cy5-labeled Streptavidin and Cy5-labeled Tetrazine), and unincorporable dNPPCP-Cy3 nucleotides (dAPPCP-Cy3, dCPPCP-Cy3, dGPPCP-Cy3, dTPPCP-Cy3) to perform 1-color DNA SBS. Step 1, Addition of DNA polymerase and the four nucleotide analogues (dATP-7-SS-Blocker-Biotin, dGTP-7-SS-Blocker-TCO, dTTP-5-SS-Blocker-Azo-TCO, dCTP-5-SS-Blocker-Azo-Biotin) to the immobilized primed DNA template enables the incorporation of the complementary nucleotide analogue to the growing DNA strand to terminate DNA synthesis. Optional Step, Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dCTP) to the immobilized primed DNA template enables the incorporation of the complementary 3′-O—SS-nucleotide analogue to the subset of growing DNA strands that were not extended with any of the Blocker-AcceptorDye containing Anchor dNTPs in step 1. The growing DNA strands are terminated with one of the four Blocker-Anchor labeled nucleotide analogues (A, C, G, T) or the same one of the four nucleotide analogues (A, C, G, T) without anchor. Step 2, after washing to remove remaining nucleotides and polymerase, Cy3-labeled unincorporable nucleotides (dNPPCP-Cy3) are added and imaging is performed with excitation of the Cy3 to obtain background Cy5 emission. Step 3, labeling with Streptavidin-Cy5 to attach the dye to the biotin-containing nucleotide analogues. The dye will bind specifically to the A and C nucleotide analogues, but not the G and T analogues. Step 4, After washing away remaining free label and excess nucleotides, addition of the four dNPPCP-Cy3 nucleotides and imaging results in detection of Cy5 emission signal due to FRET after excitation of Cy3, indicating incorporation of either A or C. Step 5, labeling with Tetrazine-Cy5 to attach the dye to the TCO-containing nucleotide analogues. The dye will bind specifically to the G and T nucleotide analogues, but not the A and C analogues. Step 6, After washing away remaining free label and excess nucleotides and addition of the four dNPPCP-Cy3 nucleotides, detection of new Cy5 signal after excitation of Cy3 indicates incorporation of either G or T. Next, in Step 7, treatment of the DNA products with sodium dithionite cleaves the Azo linker, leading to the removal of acceptor dye on T and C. Step 8, After washing away cleaved dye and re-addition of dNPPCP-Cy3 nucleotides, imaging for the presence of Cy5 fluorescence after excitation of Cy3 is carried out. In this step, if we have already determined that the incorporated nucleotide could be A or C, loss of Cy5 fluorescence would reveal it to be C, while remaining fluorescence would reveal it to be A. Similarly, for signals previously determined as G or T, loss of Cy5 fluorescence would indicate incorporation of T specifically while remaining fluorescence would indicate incorporation of G. Next, in Step 9, treatment of the DNA products with THP cleaves the SS linker, leading to the removal of the blockers and remaining Cy3 dye, and also restores the 3′-OH group on any growing strands extended with a 3′-O—SS(DTM)-dNTP in the optional chase step. Step 10, after washing away THP, an optional dNPPCP-Cy3 addition and imaging step will confirm all dyes have been removed, in preparation for the next cycle of sequencing. If this optional step is performed, it is necessary to carry out an additional wash to remove any remaining dNPPCP-Cy3. Example structures of FRET-acceptor dye labeled NRTs are presented in FIG. 7 and examples of FRET-donor dye labeled unincorporable nucleotides are presented in FIG. 13. Clusters of acceptor dyes on the nucleotides and/or donor dyes on the polymerase can also be used to amplify the energy transfer signal.

FIGS. 31A-31B: Single Color SBS with Acceptor Dye on Incoming Virtual Terminator, Donor Dye on Unincorporable Nucleotide Binding Transiently at Next Position, Using 1 Anchor and 2 Cleavable Linkers (Scheme U6). Use of dNTP-Cleavable Linker-Blocker-Dyes [dNTP-DTM(SS)-Blocker-Dye (dATP-7-SS-Blocker-Cy5), dNTP-DTM(SS)-Blocker-Azo-Dye (dTTP-5-SS-Blocker-Azo-Cy5)], dNTP-Cleavable Linker-Blocker-Anchors [dNTP-DTM(SS)-Blocker-Anchor (dGTP-7-SS-Blocker-Biotin), dNTP-DTM(SS)-Blocker-Azo-Anchor (dCTP-5-SS-Blocker-Azo-Biotin)], the corresponding Dye Labeled Binding Molecule (Cy5-labeled Streptavidin) and unincorporable dNPPCP-Cy3 nucleotides (dAPPCP-Cy3, dCPPCP-Cy3, dGPPCP-Cy3 and dTPPCP-Cy3) to perform 1-color DNA SBS. Step 1, Addition of DNA polymerase and the four nucleotide analogues (dATP-7-SS-Blocker-Cy5, dTTP-5-SS-Blocker-Azo-Cy5, dGTP-7-SS-Blocker-Biotin, dCTP-5-SS-Blocker-Azo-Biotin) to the immobilized primed DNA template enables the incorporation of the complementary nucleotide analogue to the growing DNA strand to terminate DNA synthesis. Optional Step, Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the Blocker-Dye or Blocker-Anchor dNTPs in step 1. The growing DNA strands are terminated with one of the four Blocker-Dye or Blocker-Anchor labeled nucleotide analogues (A, C, G, T) or the same one of the four nucleotide analogues (A, C, G, T) without dye or anchor. Step 2, after washing to remove remaining nucleotides and polymerase, DNA polymerase and the four dNPPCP-Cy3 unincorporable nucleotides are added and imaging is performed with excitation of the Cy3 to obtain Cy5 emission due to incorporation of either the A or T nucleotide analogue. Step 3, labeling with Streptavidin-Cy5 to attach the dye to the biotin-containing nucleotide analogues. The dye will bind specifically to the C and G nucleotide analogues. Step 4, After washing away remaining free label and excess nucleotides, DNA polymerase and the dNPPCP-Cy3 nucleotides are again added and imaging results in detection of Cy5 emission signal due to FRET after excitation of Cy3, indicating incorporation of either C or G. Next, in Step 5, treatment of the DNA products with sodium dithionite cleaves the azo linker, leading to the removal of acceptor dye on T and C. Step 6, After washing away cleaved dye, DNA polymerase and the dNPPCP-Cy3 nucleotides are again added and imaging for the presence of Cy5 fluorescence after excitation of Cy3 is carried out. In this step, if we have already determined that the incorporated nucleotide could be A or T, loss of Cy5 fluorescence would reveal it to be T, while remaining fluorescence would reveal it to be A. Similarly, for signals previously determined as C or G, loss of Cy5 fluorescence would indicate incorporation of C specifically while remaining fluorescence would indicate incorporation of G. Next, in Step 7, treatment of the DNA products with THP cleaves the SS linker, leading to the removal of the blockers and remaining Cy3 dye, and also restores the 3′-OH group on any growing strands extended with a 3′-O—SS(DTM)-dNTP in the optional chase step. Step 8, after washing away THP, an optional addition of polymerase and dNPPCP-Cy3 nucleotides is carried out and imaging will confirm all dyes have been removed, in preparation for the next cycle of sequencing. If this optional step is performed, it is necessary to carry out an additional wash to remove the remaining polymerase and dNPPCP-Cy3 nucleotides. Structures of modified nucleotides used in this scheme are shown in FIG. 8 and examples of FRET-donor dye labeled unincorporable nucleotides are presented in FIG. 13. Clusters of acceptor dyes on the nucleotides and/or donor dyes on the polymerase can also be used to amplify the energy transfer signal.

FIGS. 32A-32B: Single Color SBS with Acceptor Dye on Incoming NRT, Donor Dye on Unincorporable Nucleotide Binding Transiently at Next Position, Using 4 Cleavable Linkers (Scheme U7). Use of 3′-DTM(SS)-dNTP-Cleavable Linker-Dyes (3′-SS-dATP-7-Allyl-Cy5, 3′-SS-dTTP-5-SS-Cy5, 3′-SS-dCTP-5-Azo-Cy5, 3′-SS-dGTP-7-(2-Nitrobenzyl)-Cy5), and four dNPPCP-Cy3 unincorporable nucleotides (dAPPCP-Cy3, dCPPCP-Cy3, dGPPCP-Cy3, dTPPCP-Cy3) to perform 1-color DNA SBS. Step 1, Addition of DNA polymerase and the four nucleotide analogues (3′-SS-dATP-7-Allyl-Cy5, 3′-SS-dTTP-5-SS-Cy5, 3′-SS-dCTP-5-Azo-Cy5, 3′-SS-dGTP-7-(2-Nitrobenzyl)-Cy5) to the immobilized primed DNA template enables the incorporation of the complementary nucleotide analogue to the growing DNA strand to terminate DNA synthesis. Optional Step (not shown), Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the dye labeled dNTPs in step 1. The growing DNA strands are terminated with one of the four dye labeled nucleotide analogues (A, C, G, T) or the same one of the four nucleotide analogues (A, C, G, T) without dye. Step 2, after washing away the unincorporated dye labeled nucleotides, DNA polymerase and the four dNPPCP-Cy3 nucleotides are added and detection of the unique FRET signal confirms incorporation (Cy5 emission signal with excitation of Cy3), but does not indicate which nucleotide was incorporated. Step 3, cleavage of Allyl linker by adding Pd(0) to the elongated DNA strands results in removal of Cy5 from incorporated A. Step 4, after washing away the cleaved dyes, DNA polymerase and dNPPCP-Cy3 nucleotides are added again and a second round of imaging is performed. Loss of Cy5 signal after Cy3 excitation indicates incorporation of A. Step 5, cleavage of Azo linker by adding sodium dithionite (Na₂S₂O₄) to the elongated DNA strands results in removal of Cy5 from incorporated C. Step 6, after washing away the cleaved dyes and re-addition of DNA polymerase and dNPPCP-Cy3 nucleotides, a third round of imaging is performed. Loss of Cy5 signal after Cy3 excitation indicates incorporation of C. Step 7, cleavage of 2-nitrobenzyl linker by treating the elongated DNA strands with 340 nm light results in removal of Cy5 from incorporated G. Step 8, after washing away the cleaved dyes, a fourth round of DNA polymerase and dNPPCP-Cy3 addition and imaging is performed. Loss of Cy5 signal after Cy3 excitation indicates incorporation of G. Step 9, cleavage of SS linker by adding THP to the elongated DNA strands results in removal of Cy5 from incorporated T and also restores the 3′-OH group on any growing strands extended with a 3′-O—SS(DTM)-dNTP in the optional chase step. Step 10, after washing away the cleaved dyes, an optional final round of DNA polymerase and dNPPCP-Cy3 addition and imaging is performed. Loss of Cy5 signal after Cy3 excitation confirms incorporation of T. If this optional step is performed, it is necessary to carry out an additional wash to remove the remaining dNPPCP-Cy3. The DNA products are ready for the next cycle of the DNA sequencing reaction. Example structures of FRET-acceptor dye labeled NRTs are presented in FIG. 9 and examples of FRET-donor dye labeled unincorporable nucleotides are presented in FIG. 13. Clusters of acceptor dyes on the nucleotides and/or donor dyes on the polymerase can also be used to amplify the energy transfer signal.

FIGS. 33A-33B: Single Color SBS with Acceptor Dye on Incoming NRT, Donor Dye on Unincorporable Nucleotide Binding Transiently at Next Position, Using 4 Anchors (Scheme U8). Use of 3′-DTM(SS)-dNTP-Cleavable Linker-Dyes (3′-SS-dATP-7-SS-Biotin, 3′-SS-dTTP-5-SS-Tetrazine, 3′-SS-dCTP-5-SS-TCO, 3′-SS-dGTP-7-SS-DBCO), the corresponding dye labeled Anchor Binding Molecules (Streptavidin-Cy5, TCO-Cy5, Tetrazine-Cy5 and N₃-Cy5), and the unincorporable dNPPCP-Cy3 nucleotide analogues (dAPPCP-Cy3, dCPPCP-Cy3, dGPPCP-Cy3 and dTPPCP-Cy3) to perform 1-color DNA SBS. Step 1, Addition of DNA polymerase and the four nucleotide analogues (3′-SS-dATP-7-SS-Biotin, 3′-SS-dTTP-5-SS-Tetrazine, 3′-SS-dCTP-5-SS-TCO, 3′-SS-dGTP-7-SS-DBCO) to the immobilized primed DNA template enables the incorporation of the complementary nucleotide analogue to the growing DNA strand to terminate DNA synthesis. Optional Step (not shown), Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the anchor labeled dNTPs in step 1. The growing DNA strands are terminated with one of the four anchor labeled nucleotide analogues (A, C, G, T) or the same one of the four nucleotide analogues (A, C, G, T) without anchor. Step 2, after washing away the unincorporated anchor labeled nucleotides, DNA polymerase and the four dNPPCP-Cy3 unincorporable nucleotides are added and imaging is carried out to reveal background FRET signal (Cy5 emission signal with excitation of Cy3). Step 3, labeling with Streptavidin-Cy5 to attach the dye to the biotin-containing nucleotide analogues. The dye will bind specifically to the A nucleotide analogue. Step 4, after washing away remaining free label and excess nucleotides, DNA polymerase and the dNPPCP-Cy3 nucleotides are again added and a second round of imaging is performed. Appearance of Cy5 signal after Cy3 excitation indicates incorporation of A. Step 5, labeling with Tetrazine-Cy5 to attach the dye to the TCO-containing nucleotide analogues. The dye will bind specifically to the C nucleotide analogue. Step 6, after washing away remaining free label and excess nucleotides, DNA polymerase and the dNPPCP-Cy3 nucleotides are again added and a third round of imaging is performed. Appearance of new Cy5 signal after Cy3 excitation indicates incorporation of C. Step 7, labeling with TCO-Cy5 to attach the dye to the Tetrazine-containing nucleotide analogues. The dye will bind specifically to the T nucleotide analogue. Step 8, after washing away free label and excess nucleotides, DNA polymerase and the dNPPCP-Cy3 nucleotides are again added and a fourth round of imaging is performed. Appearance of new Cy5 signal after Cy3 excitation indicates incorporation of T. Step 9, labeling with N₃-Cy5 to attach the dye to the DBCO-containing nucleotide analogues. The dye will bind specifically to the G nucleotide analogue. Step 10, after washing away free label and excess nucleotides, DNA polymerase and the dNPPCP-Cy3 nucleotides are again added and a fifth round of imaging is performed. Appearance of new Cy5 signal after Cy3 excitation indicates incorporation of G. Step 11, cleavage of SS linker by adding THP to the elongated DNA strands restores the 3′-OH group on these nucleotide analogues as well as on any growing strands extended with a 3′-O—SS(DTM)-dNTP in the optional chase step. Step 12, after washing away the cleaved dyes, a optional final round of polymerase and dNPPCP-Cy3 addition and imaging is performed. If this optional step is performed, it is necessary to carry out an additional wash to remove the remaining polymerase and nucleotide analogues. The DNA products are ready for the next cycle of the DNA sequencing reaction. Structures of nucleotides used in this scheme are presented in FIG. 10 and examples of FRET-donor dye labeled unincorporable nucleotides are presented in FIG. 13. Clusters of acceptor dyes on the nucleotides and/or donor dyes on the polymerase can also be used to amplify the energy transfer signal.

FIGS. 34A-34B: Single Color SBS with Acceptor Dye on Incoming NRT, Donor Dye on Unincorporable Nucleotide Binding Transiently at Next Position, Using 2 Anchors and 2 Cleavable Linkers (Scheme U9). Use of 3′-DTM(SS)-dNTP-DTM(SS)-Anchor (3′-SS-dATP-7-SS-Biotin, 3′-SS-dGTP-7-SS-TCO), DTM(SS)-dNTP-Azo-Anchors (3′-SS-dTTP-5-Azo-TCO, 3′-SS-dCTP-5-SS-Azo-Biotin), the corresponding Dye Labeled Binding Molecules (Cy5-labeled Streptavidin and Cy5-labeled Tetrazine), and unincorporable nucleotides dNPPCP-Cy3 (dAPPCP-Cy3, dCPPCP-Cy3, dGPPCP-Cy3, dTPPCP-Cy3) to perform 1-color DNA SBS. Step 1, Addition of DNA polymerase and the four nucleotide analogues (3′-SS-dATP-7-SS-Biotin, 3′-SS-dGTP-7-SS-TCO, 3′-SS-dTTP-5-Azo-TCO, 3′-SS-dCTP-5-SS-Azo-Biotin) to the immobilized primed DNA template enables the incorporation of the complementary nucleotide analogue to the growing DNA strand to terminate DNA synthesis. Optional Step, Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the Anchor labeled NRTs in step 1. The growing DNA strands are terminated with one of the four Anchor labeled NRTs (A, C, G, T) or the same one of the four nucleotide analogues (A, C, G, T) without dye. Step 2, after washing to remove remaining nucleotides and polymerase, Cy3-labeled unincorporable nucleotides (dNPPCP-Cy3) are added and imaging is performed with excitation of the Cy3 to obtain background Cy5 emission. Step 3, labeling with Streptavidin-Cy5 to attach the dye to the biotin-containing nucleotide analogues. The dye will bind specifically to the A and C nucleotide analogues, but not the G and T analogues. Step 4, After washing away remaining free label and excess nucleotides, addition of DNA polymerase and the set of dNPPCP-Cy3 nucleotides followed by imaging results in detection of Cy5 emission signal due to FRET after excitation of Cy3, indicating incorporation of either A or C. Step 5, labeling with Tetrazine-Cy5 to attach the dye to the TCO-containing nucleotide analogues. The dye will bind specifically to the G and T nucleotide analogues, but not the A and C analogues. Step 6, After washing away remaining free label and excess nucleotides and re-addition of DNA polymerase and the dNPPCP-Cy3 nucleotides, detection of new Cy5 signal after excitation of Cy3 indicates incorporation of either G or T. Next, in Step 7, treatment of the DNA products with sodium dithionite cleaves the azo linker, leading to the removal of acceptor dye on T and C. Step 8, After washing away cleaved dye and re-addition of DNA polymerase and the dNPPCP-Cy3 nucleotides, imaging for the presence of Cy5 fluorescence after excitation of Cy3 is carried out. In this step, if it has already been determined that the incorporated nucleotide could be A or C, loss of Cy5 fluorescence would reveal it to be C, while remaining fluorescence would reveal it to be A. Similarly, for signals previously determined as G or T, loss of Cy5 fluorescence would indicate incorporation of T specifically while remaining fluorescence would indicate incorporation of G. Next, in Step 9, treatment of the DNA products with THP cleaves the 3′ blocking group and the SS linkers, restoring the 3′-OH and removing any remaining Cy5, and also restores the 3′-OH group on any growing strands extended with a 3′-O—SS(DTM)-dNTP in the optional chase step. Step 10, after washing away THP, an optional DNA polymerase and dNPPCP-Cy3 addition and imaging step will confirm all dyes have been removed, in preparation for the next cycle of sequencing. If this optional step is performed, it is necessary to carry out an additional wash to remove the remaining dNPPCP-Cy3. Example structures of FRET-acceptor dye labeled NRTs are presented in FIG. 11 and examples of FRET-donor dye labeled unincorporable nucleotides are presented in FIG. 13. Clusters of acceptor dyes on the nucleotides and/or donor dyes on the polymerase can also be used to amplify the energy transfer signal.

FIGS. 35A-35B: Single Color SBS with Acceptor Dye on Incoming NRT, Donor Dye on Unincorporable Nucleotide Binding Transiently at Next Position, Using 2 Anchors and 2 Cleavable Linkers (Scheme U10). Use of 3′-DTM(SS)-dNTP-Cleavable Linker-Dyes [3′-DTM(SS)-dNTP—SS-Dye (3′-DTM(SS)-dATP-7-SS-Cy5), 3′-DTM(SS)-dNTP-Azo-Dye (3′-DTM(SS)-dTTP-5-Azo-Cy5)], 3′-DTM(SS)-dNTP-Cleavable Linker-Anchors [3′-DTM(SS)-dNTP-SS-Anchor (3′-DTM(SS)-dGTP-7-SS-Biotin), 3′-DTM(SS)-dNTP-Azo-Anchor (3′-DTM(SS)-dCTP-5-Azo-Biotin)], the corresponding Dye Labeled Binding Molecule (Cy5-labeled Streptavidin), and the unincorporable dNPPCP-Cy3 nucleotides (dAPPCP-Cy3, dCPPCP-Cy3, dGPPCP-Cy3 and dTPPCP-Cy3) to perform 1-color DNA SBS. Step 1, Addition of DNA polymerase and the four nucleotide analogues (3′-DTM(SS)-dATP-7-SS-Cy5, 3′-DTM(SS)-dTTP-5-Azo-Cy5, 3′-DTM(SS)-dGTP-7-SS-Biotin, 3′-DTM(SS)-dCTP-5-Azo-Biotin) to the immobilized primed DNA template enables the incorporation of the complementary nucleotide analogue to the growing DNA strand to terminate DNA synthesis. Optional Step, Chase: addition of the DNA polymerase and four 3′-O—SS(DTM)-dNTPs (3′-O-t-Butyldithiomethyl(SS)-dATP, 3′-O-t-Butyldithiomethyl(SS)-dCTP, 3′-O-t-Butyldithiomethyl(SS)-dTTP and 3′-O-t-Butyldithiomethyl(SS)-dGTP) to the immobilized primed DNA template enables the incorporation of the complementary 3′-O—SS-nucleotide analogue to the subset of growing DNA strands that were not extended with any of the Blocker-Dye or Blocker-Anchor dNTPs in step 1. The growing DNA strands are terminated with one of the four Blocker-Dye or Blocker-Anchor labeled nucleotide analogues (A, C, G, T) or the same one of the four nucleotide analogues (A, C, G, T) without dye or anchor. Step 2, after washing to remove remaining nucleotides and polymerase, DNA polymerase and the four dNPPCP-Cy3 unincorporable nucleotides are added and imaging is performed with excitation of the Cy3 to obtain Cy5 emission due to incorporation of either the A or T nucleotide analogue. Step 3, labeling with Streptavidin-Cy5 to attach the dye to the biotin-containing nucleotide analogues. The dye will bind specifically to the C and G nucleotide analogues. Step 4, After washing away remaining free label and excess nucleotides, DNA polymerase and dNPPCP-Cy3 nucleotides are added and imaging results in detection of Cy5 emission signal due to FRET after excitation of Cy3, indicating incorporation of either C or G. Next, in Step 5, treatment of the DNA products with sodium dithionite cleaves the azo linker, leading to the removal of acceptor dye on T and C. Step 6, After washing away cleaved dye, DNA polymerase and dNPPCP-Cy3 nucleotides are added and imaging for the presence of Cy5 fluorescence after excitation of Cy3 is carried out. In this step, if it has already been determined that the incorporated nucleotide could be A or T, loss of Cy5 fluorescence would reveal it to be T, while remaining fluorescence would reveal it to be A. Similarly, for signals previously determined as C or G, loss of Cy5 fluorescence would indicate incorporation of C specifically while remaining fluorescence would indicate incorporation of G. Next, in Step 7, treatment of the DNA products with THP cleaves the 3′ blocking group and the SS linkers, restoring the 3′-OH and removing any remaining Cy5, and also restores the 3′-OH group on any growing strands extended with a 3′-O—SS(DTM)-dNTP in the optional chase step. Step 8, after washing away THP, an optional DNA polymerase and dNPPCP-Cy3 nucleotide addition and imaging step will confirm all dyes have been removed, in preparation for the next cycle of sequencing. If this optional step is performed, it is necessary to carry out an additional wash to remove the remaining polymerase and nucleotide analogues. Structures of modified nucleotides used in this scheme are shown in FIG. 12 and examples of FRET-donor dye labeled unincorporable nucleotides are presented in FIG. 13. Clusters of acceptor dyes on the nucleotides and/or donor dyes on the polymerase can also be used to amplify the energy transfer signal.

### DETAILED DESCRIPTION

The currently widely used high-throughput SBS technology (Bentley et al. 2008) uses cleavable fluorescent nucleotide reversible terminator (NRT) sequencing chemistry developed previously (Ju et al. 2003; Ju et al. 2006). These cleavable fluorescent NRTs were designed based on the following rationale: each of the four nucleotides (A, C, G, T) is modified by attaching a unique cleavable fluorophore to the specific location of the base and capping the 3′-OH group with a small reversible moiety so that they are still recognized by DNA polymerase as substrates. Thus, the cleavable fluorescent NRTs involve two site modifications (Ju et al. 2003; Ju et al. 2006): a fluorescent dye to serve as a reporter group on the base and a small chemical moiety to cap the 3′-OH group to temporarily terminate the polymerase reaction after nucleotide incorporation for sequence determination. After incorporation and signal detection, the fluorophore is cleaved and the 3′-OH capping moiety removed to resume the polymerase reaction in the next cycle. These cleavable fluorescent NRTs have proved to be good substrates for reengineered polymerases and have been used extensively in next generation DNA sequencing systems (Ju et al. 2006; Bentley et al. 2008). Moreover, they enable accurate determination of homopolymer sequences, since only one base is identified in each cycle.

Designs for sequencing by synthesis (SBS) previously described in PCT/US2019/022326, which is hereby incorporated by reference in its entirety, use fluorescence resonance energy transfer (FRET) dyes in several SBS schemes, in which a donor fluorophore is excited in its absorption range, transfers energy to an acceptor fluorophore, and emission of the acceptor fluorophore is monitored. At the same time, a decrease in the detectable emission signal of the donor fluorophore can be monitored. In said SBS schemes, the donor and acceptor dyes are present on the same nucleotide in one of the following three configurations.

In the first configuration the acceptor (e.g., Cy5 or ATTO647N) is attached directly to the base and donor (e.g., Cy3 or Cy2) present on a labeling molecule which binds to an anchor on the base. Thus, the donor and acceptor are brought together during the labeling reaction.

This configuration can use 3′-blocked nucleotide reversible terminators, dideoxynucleotide triphosphates (ddNTPs), and virtual terminators with the blocking group attached to the base of the nucleotide. In the second configuration, the acceptor is attached directly to the base and donor attached to the 3′ position via an anchor and labeling molecule. Again, the donor and acceptor are brought within energy transfer distance during the labeling reaction. This configuration can use 3′-blocked nucleotide reversible terminators. The third configuration is the same as the first two, but the positions of the donor and acceptor reversed. Also described was the use of a quantum dot as a FRET donor. The energy transfer approach is most applicable to single color detection methods, though in theory 2 or even more colors can be detected with carefully designed donor/acceptor combinations (using 2 FRET systems (Donor 1→Acceptor 1/Donor 2→Acceptor 2 including in particular the case where Acceptor 1 is Donor 2) and/or using varying distances or ratios among the multiple FRET dyes. Additionally, clusters of acceptor and/or donor dyes can be used to increase the chance of FRET occurring or increase the strength of the overall signal.

In addition to placement of the donor and acceptor dyes on the same nucleotide, the donor or acceptor may be placed on a different molecule in the system, e.g., the polymerase or an adjacent nucleotide. The placement of donor dye molecules on the polymerase with acceptor dyes on either unincorporable nucleotides, reversible terminators, or natural nucleotides has also been described previously (Ju et al, WO 2017/176677 A1), and as described herein.

The invention disclosed herein provides a method of sequencing nucleic acid comprising:
a) providing at least one nucleic acid template hybridized to a primer;
b) providing a first nucleic acid polymerase and extending the primer hybridized to said at least one nucleic acid template with the first nucleic acid polymerase and a fluorescently labeled nucleotide analogue if the nucleotide analogue is complementary to a nucleotide residue which is immediately 5′ to the nucleotide residue of the nucleic acid template hybridized to the 3′ terminal nucleotide residue of the primer, wherein the nucleotide analogue is either:
   (i) a fluorescently labeled nucleotide analogue comprising a base and a fluorescent label and a blocking group attached to the base of the nucleotide analogue via a cleavable linker, wherein said blocking group prevents or greatly reduces incorporation of a subsequent nucleotide analogue into the extended primer strand, and wherein said fluorescent label comprises an energy transfer acceptor or donor dye; or
   (ii) a fluorescently labeled reversibly blocked nucleotide analogue comprising a base and a fluorescent label attached to the nucleotide analogue via a cleavable linker and a blocking group at the 3′-OH position, wherein said blocking group prevents incorporation of a subsequent nucleotide analogue into the extended primer strand, and wherein the fluorescent label comprises an energy transfer acceptor or donor dye;
c) removing the first nucleic acid polymerase and providing a second nucleic acid polymerase having an attached fluorescent label, wherein said fluorescent label comprises an energy transfer acceptor or donor dye for the energy transfer acceptor or donor dye attached to the nucleotide analogue in step b;
d) identifying the fluorescence signal due to incorporation of the fluorescently labeled nucleotide analogue onto the primer;
e) optionally extending any unextended primer with a 3′ blocked nucleotide analogue without any base modifications;
f) cleaving the label and the blocking group from any incorporated nucleotide analogue of step b);
g) wherein if no fluorescence signal is detected in step d), iteratively repeating steps b) to f) with a fluorescently labeled nucleotide analogue having a different base until the fluorescently labeled nucleotide analogue is incorporated;
h) optionally extending any unextended primer with a 3′ blocked nucleotide analogue without any base modifications if the optional chase step e) was not carried out;
i) iteratively performing steps b) to h) for each nucleotide residue of the nucleic acid template,
   thereby determining the sequence of the nucleic acid template.

The invention provides the instant method, wherein the fluorescently labeled nucleotide analogue has a blocking group attached to the base. The invention provides the instant method, wherein the fluorescently labeled nucleotide analogue has a blocking group at the 3′-OH position.

In an embodiment of the invention, the dye on the nucleotide analogue is Cy5 or ATTO647N and the dye on the second nucleic acid polymerase of step c) is Cy3. In an embodiment of the invention, the cleavable linker on the base is DTM.

In an embodiment of the invention, the 3′ blocking group of the fluorescently labeled nucleotide analogue is DTM or azidomethyl, and the cleavage is carried out with THP.

In an embodiment of the invention, dye on the fluorescently labeled nucleotide analogues is an energy transfer donor dye and the dye on the second nucleotide polymerase is an energy transfer acceptor dye, or wherein the dye on the fluorescently labeled nucleotide analogues is an energy transfer acceptor dye and the dye on the second nucleotide polymerase is an energy transfer donor dye. In an embodiment of the invention, wherein the dye on the nucleotide analogue and/or second nucleic acid polymerase is a dye cluster.

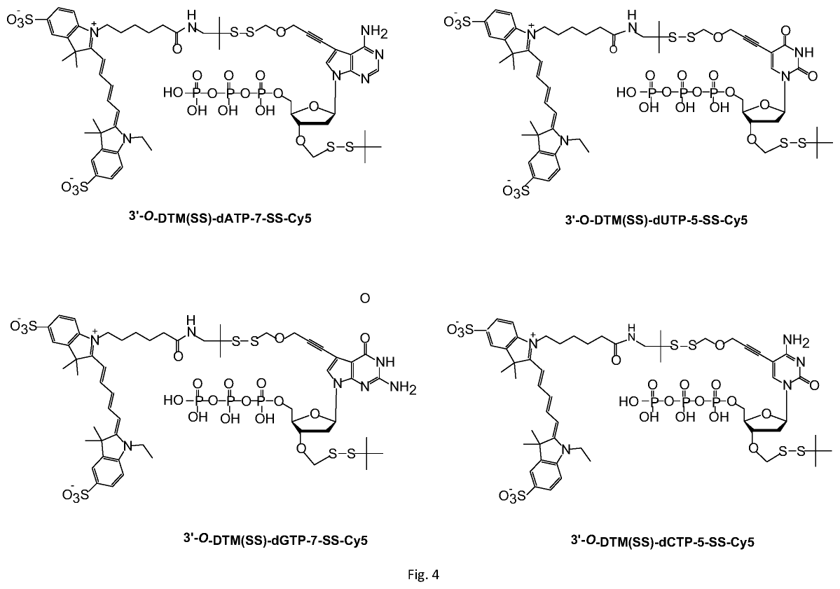

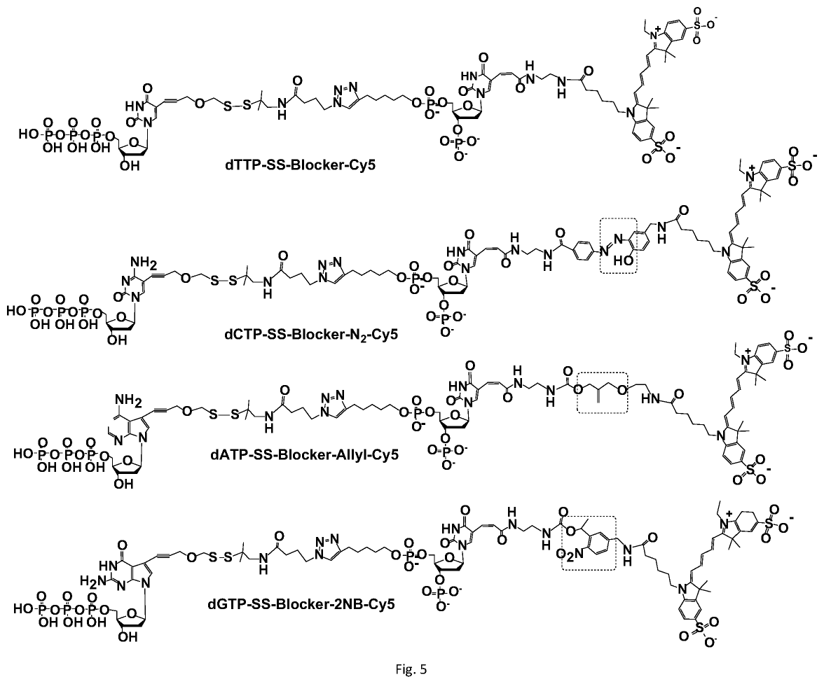

In an embodiment of the invention, the fluorescently labeled nucleotide analogue is selected from any one of the nucleotide analogues of FIG. 4 and/or FIG. 9. In an embodiment of the invention, the fluorescently labeled nucleotide analogue is selected from any one of nucleotide analogues of FIG. 5.

The invention further provides a kit comprising all the required nucleotide analogues, polymerases, cleavage agents and other reaction buffer components for carrying out the instant method.

The invention further provides a method of sequencing nucleic acid comprising:
a) providing at least one nucleic acid template hybridized to a primer;
b) providing a first nucleic acid polymerase and four different labeled nucleotide analogues (A, C, T, G) and extending the primer hybridized to said at least one nucleic acid template with the first nucleic acid polymerase and one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) fluorescently labeled nucleotide analogues comprising a base and a blocking group linked to the base via a cleavable linker and a fluorescent label linked distal to the blocking group via either an uncleavable or a different cleavable linker, wherein said blocking group prevents or greatly reduces incorporation of a subsequent nucleotide analogue into the extended primer strand, wherein each of the different nucleotide analogues (A, C, G, T) have the same fluorescent label and different cleavable linkers, and wherein said fluorescent label comprises an energy transfer acceptor or donor dye; or
   (ii) fluorescently labeled nucleotide analogues comprising a base and a fluorescent label attached to the base via a cleavable linker and a blocking group at the 3′-OH position, wherein said blocking group prevents incorporation of a subsequent nucleotide analogue into the extended primer strand, and wherein each of the different nucleotide analogues (A, C, G, T) have the same fluorescent label and different cleavable linkers, and wherein said fluorescent label comprises an energy transfer acceptor or donor dye;
c) removing the first nucleic acid polymerase and providing a second nucleic acid polymerase having an attached fluorescent label, wherein said fluorescent label comprises an energy transfer acceptor or donor dye for the energy transfer acceptor or donor dye attached to the nucleotide analogue incorporated in step b;
d) optionally extending any unextended primer with a 3′ blocked nucleotide analogue without any base modifications;
e) identifying the fluorescence resonance energy transfer (FRET) signal due to incorporation of the fluorescently labeled nucleotide analogue;
f) contacting the incorporated labeled nucleotide analogues with a cleaving agent that cleaves the cleavable linker to remove the label from one of the four different labeled nucleotide analogues, wherein said cleaving agent does not cleave the cleavable label from the remaining labeled nucleotide analogues;
g) replenishing the second nucleic acid polymerase and identifying any loss of FRET signal due to the cleavage carried out in step f) to partially or completely identify the incorporated nucleotide;
h) iteratively repeating steps f) and g) with a cleaving agent that cleaves the cleavable linker to remove the label from a different labeled nucleotide analogue, wherein said cleaving agent does not cleave the label from the remaining labeled nucleotide analogues;
i) determining the labeled nucleotide analogue incorporated in step b) by comparing the results obtained in the multiple iterations of step g); and
j) cleaving the blocking group and at the same time cleaving any remaining fluorescent labels from the extended primers, and iteratively carrying out steps b to j to obtain the sequence of the nucleic acid template.

In an embodiment, the fluorescently labeled nucleotide analogue has a blocking group attached to the base. In an embodiment, the fluorescently labeled nucleotide analogue has a blocking group at the 3′-OH position.

In an embodiment, the dye on the nucleotide analogue is Cy5 or ATTO647N and the dye on the second nucleic acid polymerase of step c) is Cy3. In an embodiment, the dyes comprise dye clusters.

In an embodiment of the instant method, the four labeled nucleotide analogues of step b) (i) each comprise the same type of cleavable linker linking the blocking group to the base, and three of the four labeled nucleotide analogues comprise a different cleavable linker linking the fluorescent label distal to the blocking group. In an embodiment, the cleavable linkers comprise DTM, azo, allyl and 2-nitrobenzyl and the cleaving agents comprise THP, sodium dithionite, Pd(0) and UV light (˜340 nm) respectively.

In an embodiment of the instant method, the four labeled nucleotide analogues of step b) (ii), each comprise a different cleavable linker linking the fluorescent label to the base, and wherein the cleaving agent the cleaves one of the cleavable linkers also cleaves the blocking group at the 3′-OH position, and wherein the cleaving agent that cleaves the blocking group at the 3′-OH contacts the incorporated labeled nucleotide analogue in the final iteration of step f). In an embodiment, the cleavable linkers comprise DTM, azo, allyl and 2-nitrobenzyl and the cleaving agents comprise THP, sodium dithionite, Pd(0) and UV light (˜340 nm) respectively.

In an embodiment of the instant method, the dye on the fluorescently labeled nucleotide analogues is an energy transfer donor dye and the dye on the second nucleotide polymerase is an energy transfer acceptor dye, or wherein the dye on the fluorescently labeled nucleotide analogues is an energy transfer acceptor dye and the dye on the second nucleotide polymerase is an energy transfer donor dye.

In an embodiment of the instant method, the four labeled nucleotide analogues consist of those found in FIG. 5. In another embodiment, the four labeled nucleotide analogues consist of those found in FIG. 9.

The invention further provides a kit comprising all the required nucleotide analogues, polymerases, cleavage agents and other reaction buffer components for carrying out the instant method.

The invention further provides a method of sequencing nucleic acid comprising:
a) providing at least one nucleic acid template hybridized to a primer;
b) providing a first nucleic acid polymerase and four different anchor labeled nucleotide analogues (A, C, T, G) and extending the primer hybridized to said at least one nucleic acid template with the first nucleic acid polymerase and one of the anchor labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different anchor labeled nucleotide analogues are either:
   (i) anchor labeled nucleotide analogues each comprising a base, a blocking group linked to the base via a cleavable linker, and an anchor linked to the base via an uncleavable linker distal to the blocking group, wherein said blocking group prevents or greatly reduces incorporation of a subsequent nucleotide analogue into the extended primer strand, wherein each different anchor labeled nucleotide analogue (A, C, G, T) has a different anchor and the same cleavable linker; or
   (ii) anchor labeled nucleotide analogues each comprising a base, an anchor linked to the base via a cleavable linker, and a blocking group at the 3′-OH position, wherein said blocking group prevents incorporation of a subsequent nucleotide analogue into the extended primer strand, and wherein each different anchor labeled nucleotide analogue (A, C, G, T) has different anchor from the remaining anchor labeled nucleotide analogues and the same linker;
c) removing the first nucleic acid polymerase and providing a second nucleic acid polymerase having an attached fluorescent label, wherein said fluorescent label attached to the polymerase is an energy transfer donor or acceptor dye;
d) optionally extending any unextended primer with a 3′ blocked nucleotide analogue without any base modifications;
e) identifying any background fluorescence resonance energy transfer (FRET) signal;
f) labeling any primer extension products with a fluorescently labeled anchor binding molecule specific for one of the four anchors of the nucleotide analogues of step b), wherein the anchor binding molecule comprises a fluorescent label, wherein said fluorescent label is an energy transfer donor or acceptor dye for the energy transfer donor or acceptor dye attached to the second nucleic acid polymerase;
g) optionally replenishing the second nucleic acid polymerase and identifying any fluorescence resonance energy transfer (FRET) signal due to the anchor binding molecule binding to the anchor labeled nucleotide analogue incorporated in step b);
h) iteratively repeating steps f) and g) with a fluorescently labeled anchor binding molecule specific for each of the remaining anchor labeled nucleotide analogues one by one, wherein the same fluorescent dye is attached to all four anchor binding molecules;
i) determining the specific nucleotide analogue incorporated by comparing the results obtained in the multiple iterations of step g);
j) contacting the incorporated with a cleaving agent to cleave the blocking group and the anchor and fluorescent labels from the incorporated nucleotide analogue of step b); and
iteratively carrying out steps b) to j) to thereby obtain the sequence of the nucleic acid template.

In an embodiment, the four anchor labeled nucleotide analogues are those from step b) i). In another embodiment, the four anchor labeled nucleotide analogues are those from step b) ii).

In an embodiment, the dyes each comprise dye clusters.

In an embodiment of the instant method, the four anchor labeled nucleotide analogues of step b) (i) each comprise a blocking group attached to the base via the same cleavable linker, a different anchor attached distal to the blocking group via an uncleavable linker, and wherein the anchor of each anchor labeled nucleotide analogue binds to a different anchor binding molecule. In an embodiment, each of the four different anchors independently comprises one of biotin, TCO, DBCO or tetrazine and each of the fluorescently labeled anchor binding molecules independently comprises one of streptavidin, tetrazine, azido and TCO respectively.

In an embodiment of the instant method, the four anchor labeled nucleotide analogues of step b) ii) each comprise a different anchor attached to the base via the same cleavable linker, and wherein the anchor of each anchor labeled nucleotide analogue binds to a different anchor binding molecule. In an embodiment, each of the four different anchors independently comprises one of biotin, TCO, DBCO and tetrazine, and wherein each of the fluorescently labeled anchor binding molecules independently comprises one of streptavidin, tetrazine, azido and TCO respectively.

In an embodiment, the dye on the fluorescently labeled anchor binding molecules is an energy transfer donor dye and the dye on the second nucleotide polymerase is an energy transfer acceptor dye, or wherein the dye on the fluorescently labeled anchor binding molecules is an energy transfer acceptor dye and the dye on the second nucleotide polymerase is an energy transfer donor dye.

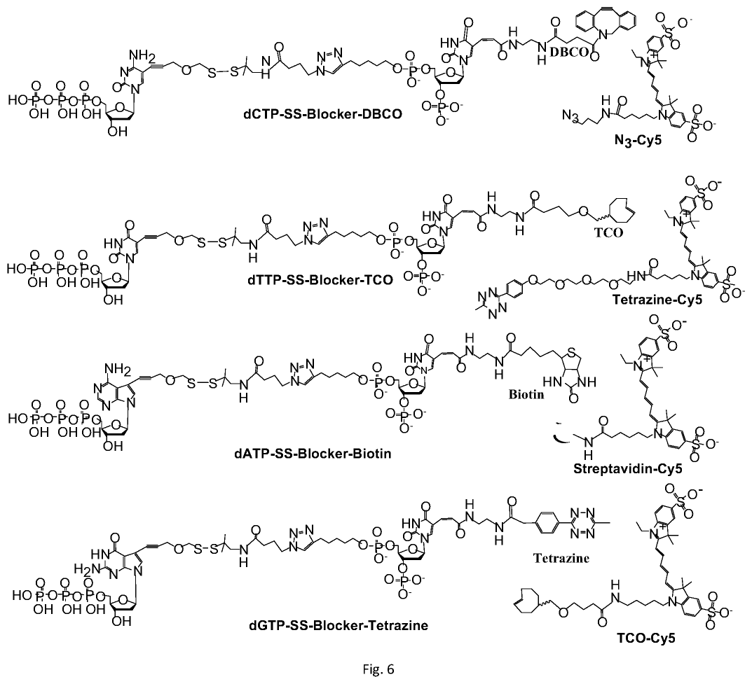

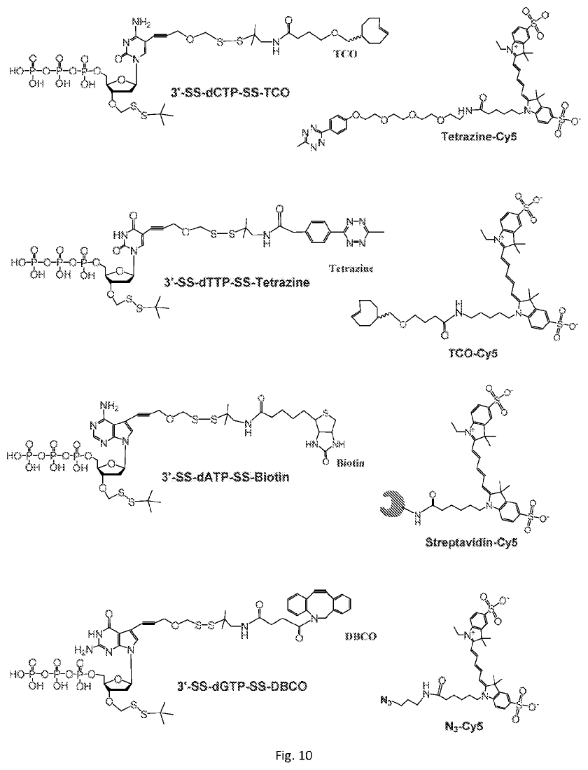

In an embodiment, the anchor labeled nucleotide analogues and corresponding anchor binding molecules are those of FIG. 6. In another embodiment, the anchor labeled nucleotide analogues and corresponding anchor binding molecules are those of FIG. 10.

The invention further provides a kit comprising all the required nucleotide analogues, polymerases, labeled anchor binding molecules, cleavage agents and other reaction buffer components for carrying out the instant method.

The invention further provides a method of sequencing nucleic acid comprising:
a) providing at least one nucleic acid template hybridized to a primer;
b) providing a first nucleic acid polymerase and four different anchor labeled nucleotide analogues (A, C, T, G) and extending the primer hybridized to said at least one nucleic acid template with the first nucleic acid polymerase and one of the anchor labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different anchor labeled nucleotide analogues are either:
   (i) anchor labeled nucleotide analogues each comprising a base and a blocking group linked to the base via the same cleavable linker, wherein said blocking group prevents or greatly reduces incorporation of a subsequent nucleotide analogue into the extended primer strand, wherein two of the different anchor labeled nucleotide analogues (A, C, G, T) comprise the same anchor and the remaining two different anchor labeled nucleotide analogues comprise the same anchor, wherein the anchor of two of the anchor labeled nucleotide analogues is attached distal to the blocking group via an uncleavable linker and the anchor of each of two of the anchor labeled nucleotide analogues is attached distal to the blocking group via the same cleavable linker; or
   (ii) anchor labeled nucleotide analogues each comprising a base, an anchor attached to the base via a cleavable linker, and a blocking group at the 3′-OH position, wherein said blocking group prevents incorporation of a subsequent nucleotide analogue into the extended primer strand, wherein two of the different anchor labeled nucleotide analogues (A, C, G, T) comprise the same anchor and the remaining two different anchor labeled nucleotide analogues comprise the same anchor, wherein the cleavable linker of two of the anchor labeled nucleotide analogues is the same, and wherein the cleavable linker of the remaining two anchor labeled nucleotide analogues is the same and different cleavable groups;
c) removing the first nucleic acid polymerase and providing a second nucleic acid polymerase having an attached fluorescent label, wherein said fluorescent label attached to the polymerase is an energy transfer donor or acceptor dye;
d) optionally extending any unextended primer with a 3′ blocked nucleotide analogue without any base modifications;
e) identifying any background fluorescence resonance energy transfer (FRET) signal;
f) labeling any primer extension products with a fluorescently labeled anchor binding molecule specific for one of the anchors of the nucleotide analogues of step b), wherein the anchor binding molecule comprises a fluorescent label, wherein said fluorescent label is an energy transfer donor or acceptor dye for the energy transfer donor or acceptor dye attached to the second nucleic acid polymerase;
g) identifying newly generated FRET signals due to the labeling in step f) to partially identify the incorporated nucleotide analogue of step b);
h) repeating steps e and f with a second fluorescently labeled anchor binding molecule specific for the second anchor;
i) cleaving the dye from the fluorescently labeled nucleotides with a specific cleavable agent that cleaves one of the cleavable linkers but does not cleave any remaining linker;
j) optionally replenishing the second nucleic acid polymerase and identifying loss of FRET signal due to the cleavage carried out in step i);
k) determining the specific nucleotide analogue incorporated by comparing the results obtained in steps g) and j);
l) cleaving the blocking group and at the same time cleaving the remaining anchors and fluorescent labels from any extended primers;
and iteratively carrying out steps b) to l) to obtain the sequence of the nucleic acid template.

In an embodiment of the instant method, the four anchor labeled nucleotide analogues are those from step b) i). In another embodiment, the four anchor labeled nucleotide analogues are those from step b) ii). In an embodiment, the dyes comprise dye clusters.

In an embodiment of the instant method, the four anchor labeled nucleotide analogues (A, C, G, T) comprise two different cleavable linkers and two different anchors (Anchor 1 and Anchor 2), one cleavable linker (Cleavable Linker 1) between the blocking group and the base of all four of the anchor labeled nucleotide analogues, and a different cleavable linker (Cleavable Linker 2) distal to the blocking group linking the anchor to the blocking group of two of the nucleotide analogues, thereby producing a set of the 4 nucleotides or nucleotide analogues, one consisting of Cleavable Linker 1 with Anchor 1, one consisting of Cleavable Linker 1 with Anchor 2, one consisting of Cleavable Linker 1, Cleavable Linker 2, and Anchor 1, and the last consisting of Cleavable Linker 1, Cleavable Linker 2, and Anchor 2, wherein cleavage of Cleavable Linker 2 and detection of fluorescence signals is carried out prior to the final cleavage of Cleavable Linker 1. In an embodiment, the cleavable linkers comprise DTM and azo, the cleaving agents comprise THP and sodium dithionite respectively, the anchors comprise biotin and TCO, and the fluorescently labeled anchor binding molecules comprise streptavidin and tetrazine respectively.

In an embodiment of the instant method, the four different anchor labeled nucleotide analogues (A, C, G, T) comprise 2 different cleavable linkers and 2 different anchors (Anchor 1 and Anchor 2), wherein one cleavable linker (Cleavable Linker 1) links the base of a nucleotide analogue to an anchor, the other cleavable linker (Cleavable Linker 2) links the base of a nucleotide analogue to an anchor, wherein one of the anchor labeled nucleotide analogues has Cleavable Linker 1 and Anchor 1, another has Cleavable Linker 1 and Anchor 2, another has Cleavable Linker 2 and Anchor 1, and the final has Cleavable Linker 2 and Anchor 2, thereby producing a set of 4 nucleotide analogues, wherein an agent capable of cleaving Cleavable Linker 1 also removes the 3′-OH blocking group, and wherein cleavage of Cleavable Linker 2 and determination of signal is performed prior to cleavage of Cleavable Linker 1. In an embodiment, the cleavable linkers independently comprise DTM or azo, the cleaving agents comprise THP and sodium dithionite respectively, the anchors comprise biotin and TCO, and the fluorescently labeled anchor binding molecules comprise streptavidin and tetrazine respectively.

In an embodiment of the instant method, the dye on the fluorescently labeled anchor binding molecules is an energy transfer donor dye and the dye on the second nucleotide polymerase is an energy transfer acceptor dye, or wherein the dye on the fluorescently labeled anchor binding molecules is an energy transfer acceptor dye and the dye on the second nucleotide polymerase is an energy transfer donor dye.

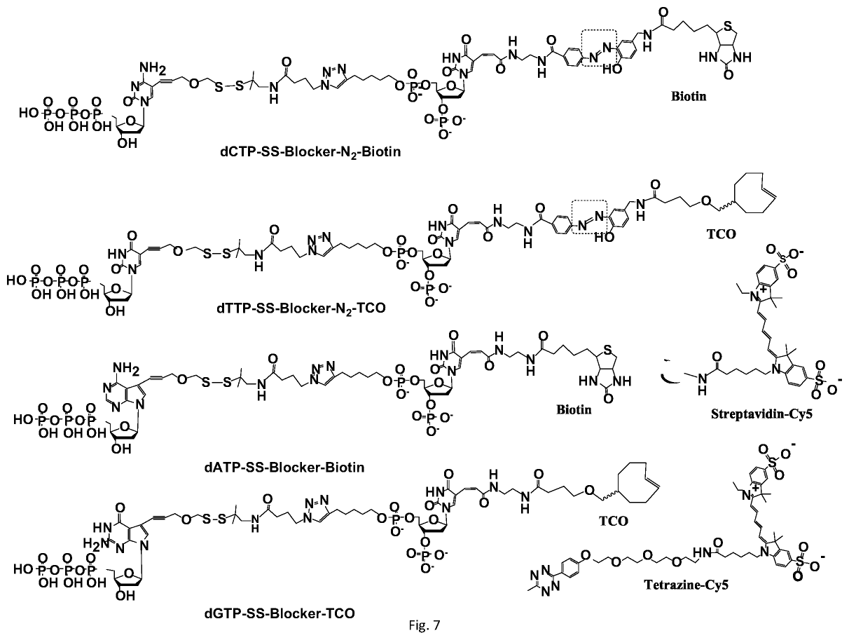

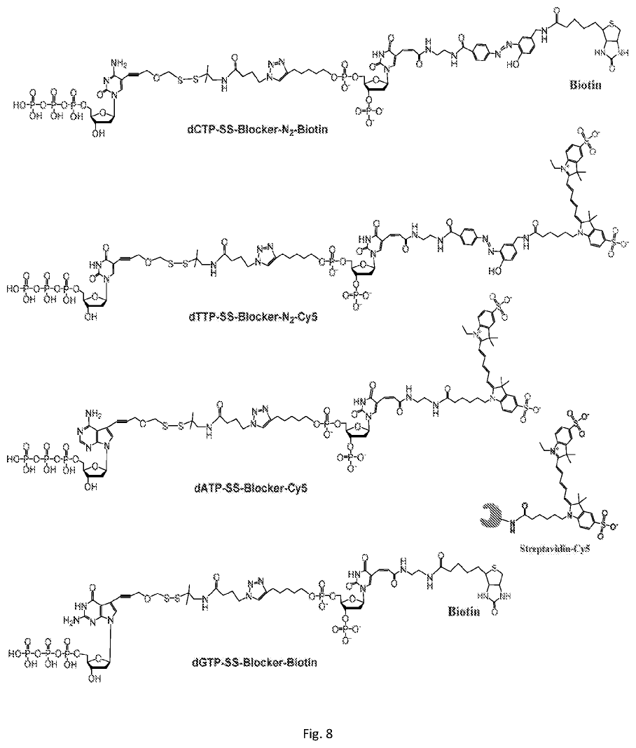

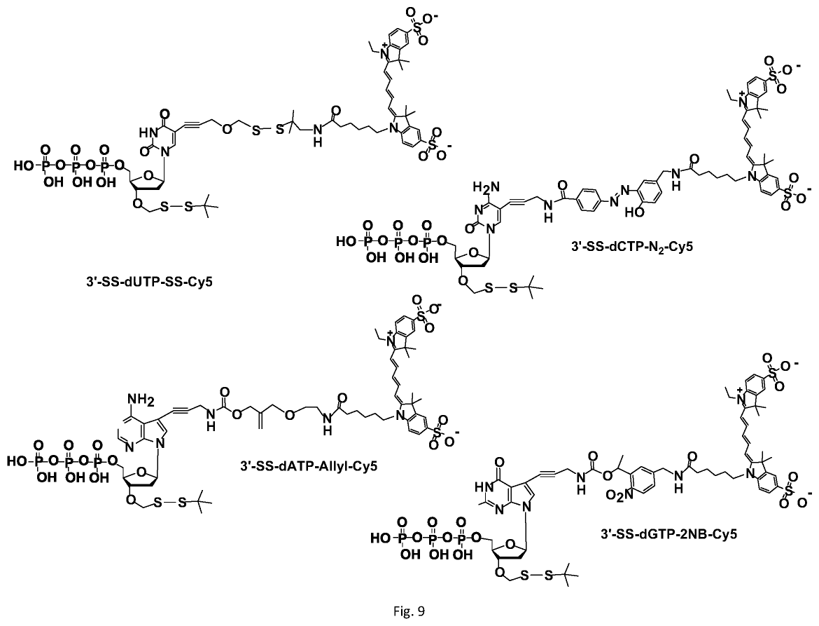

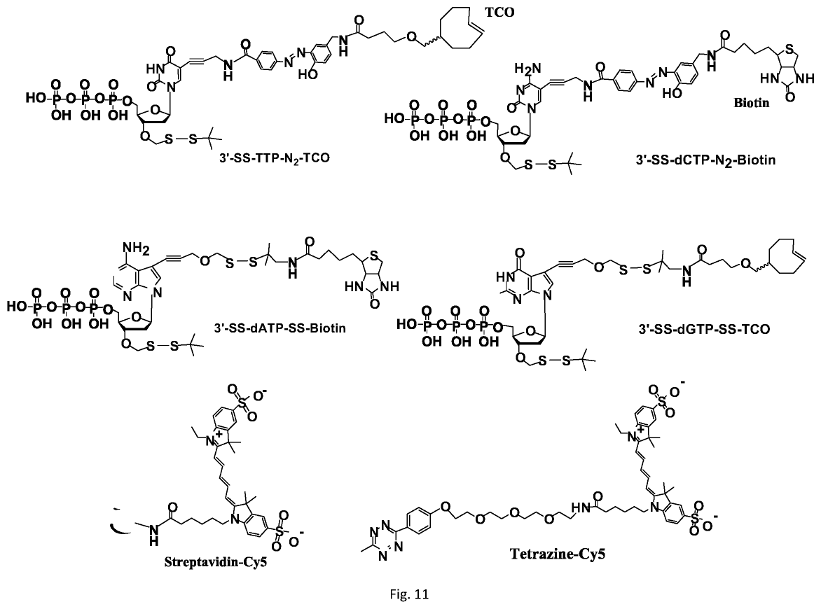

In an embodiment of the instant method, the anchor labeled nucleotide analogues and corresponding anchor binding molecules are those of FIG. 7. In an embodiment of the instant method, the anchor labeled nucleotide analogues and corresponding anchor binding molecules are those of FIG. 11.

The invention further provides a kit comprising all the required nucleotide analogues, polymerases, labeled anchor binding molecules, cleavage agents and other reaction buffer components for carrying out the instant method.

The invention further provides a method of sequencing nucleic acid comprising:
a) providing at least one nucleic acid template hybridized to a primer;
b) providing a first nucleic acid polymerase, four different labeled nucleotide analogues (A, C, T, G) and extending the primer hybridized to said at least one nucleic acid template with the first nucleic acid polymerase and one of the nucleotide analogues if the nucleotide analogue is complementary to a nucleotide residue which is immediately 5′ to the nucleotide residue of the nucleic acid template hybridized to the 3′ terminal nucleotide residue of the primer, wherein the four labeled nucleotide analogues are either:
(i) two different fluorescently labeled nucleotide analogues that comprise a base and a blocking group linked to the base via
   a first cleavable linker,
      wherein one of the fluorescently labeled nucleotide analogues comprises a fluorescent label attached distal to the blocking group via a second cleavable linker wherein said fluorescent label is an energy transfer donor or acceptor dye, and
      wherein the remaining fluorescently labeled nucleotide analogue comprises a fluorescent label attached distal to the blocking group via an uncleavable linker wherein said fluorescent label is an energy transfer donor or acceptor dye, and
   two different anchor labeled nucleotide analogues that comprise a base and a blocking group linked to the base via the first cleavable linker,
      wherein one of the anchor labeled nucleotide analogues comprises an anchor attached distal to the blocking group via the second cleavable linker, and
      wherein one the remaining anchor labeled nucleotide analogue comprises the same anchor attached distal to the blocking group via an uncleavable linker; or
(ii) two different fluorescently labeled nucleotide analogues that comprise a base and a blocking group at the 3′-OH position,
   wherein one of the fluorescently labeled nucleotide analogues comprises a fluorescent label attached to the base via a first cleavable linker wherein said fluorescent label is an energy transfer donor or acceptor dye, and
   wherein the remaining fluorescently labeled nucleotide analogue comprises a fluorescent label attached to the base via a second cleavable linker wherein said fluorescent label is an energy transfer donor or acceptor dye, and
two different anchor labeled nucleotide analogues that comprise a base and a blocking group at the 3′-OH position,
   wherein one of the anchor labeled nucleotide analogues comprises an anchor linked to the base via the first cleavable linker, and
   wherein the remaining anchor labeled nucleotide analogue comprises the same anchor linked to the base via a second cleavable linker;
c) removing the first nucleic acid polymerase and providing a second nucleic acid polymerase having an attached fluorescent label, wherein said fluorescent label attached to the polymerase is an energy transfer donor or acceptor dye for the fluorescent label of the fluorescently labeled nucleotide analogues;
d) optionally extending any unextended primer with a 3′ blocked nucleotide analogue without any base modifications;
e) identifying the fluorescence resonance energy transfer (FRET) signal due to incorporation of any fluorescently labeled nucleotide analogues;
f) labeling anchor attached primer extension products with a fluorescently labeled anchor binding molecule specific for one of the anchors, wherein the fluorescent label is the same as that on the fluorescently labeled nucleotide analogues;
g) optionally replenishing the second nucleic acid polymerase and identifying any newly generated FRET signals to partially identify the incorporated nucleotides due to the labeling carried out in step f);
h) cleaving the dye from the fluorescently labeled nucleotides with a specific cleaving agent that cleaves one of the linkers but does not cleave any remaining linkers;
i) optionally replenishing the second nucleic acid polymerase and identifying any loss of FRET signals due to the cleavage carried out in step g);
j) determining the specific nucleotide analogue incorporated by comparing the results obtained in steps g) and i);
k) cleaving the blocking group and at the same time cleaving any remaining anchors and fluorescent labels from the extended primers;
and iteratively carrying out steps b) to k) to obtain the sequence of the nucleic acid template.

In an embodiment of the instant method, the four anchor labeled nucleotide analogues are those from step b) i). In an embodiment of the instant method, the four anchor labeled nucleotide analogues are those from step b) ii). In an embodiment of the instant method, the dyes comprise dye clusters.

In an embodiment of the instant method, the four labeled nucleotide analogues comprise 2 different cleavable linkers and 2 different anchors, one cleavable linker (Cleavable Linker 1) between the blocking group and all four of the nucleotides or nucleotide analogues, and a different cleavable linker (Cleavable Linker 2) between the base and the anchors but distal to the blocking group for two of the nucleotides or nucleotide analogues, thereby producing a set of the 4 nucleotides or nucleotide analogues, one comprising Cleavable Linker 1 and Anchor 1, one comprising Cleavable Linker 1 with Anchor 2, one comprising Cleavable Linker 1, Cleavable Linker 2, and Anchor 1, and the last comprising Cleavable Linker 1, Cleavable Linker 2, and Anchor 2, wherein cleavage of Cleavable Linker 2 and detection of fluorescence signals is carried out prior to the final cleavage of Cleavable Linker 1. In an embodiment, Cleavable Linker 1 comprises DTM, Cleavable Linker 2 comprises azo, the cleaving agents comprise THP and sodium dithionite respectively, the anchors comprise biotin (Anchor 1) and TCO (Anchor 2), and the fluorescently labeled anchor binding molecules comprise streptavidin and tetrazine respectively.

In an embodiment of the instant method, the four labeled nucleotide analogues 2 different cleavable linkers and 2 different anchors, the first type of cleavable linker between one of the nucleotides or nucleotide analogues and one of the anchors, the first type of cleavable linker between the second nucleotide analogue and the second type of anchor, a second type of cleavable linker between the third nucleotide analogue and the first anchor, and the second type of cleavable linker between the fourth nucleotide analogue and the second anchor, thereby producing a set of 4 nucleotides or nucleotide analogues, wherein cleavage of the first type of cleavable linker is also capable of removing the 3′-OH blocking group, and wherein cleavage of the second type of cleavable linker and determination of signal is performed prior to cleavage of the first type of cleavable linker. In an embodiment, the cleavable linkers comprise DTM and azo, the cleaving agents comprise THP and sodium dithionite respectively, the anchors comprise biotin and TCO, and the fluorescently labeled anchor binding molecules comprise streptavidin and tetrazine respectively.

In an embodiment of the instant method, dye on the fluorescently labeled anchor binding molecules and fluorescently labeled nucleotide analogues is an energy transfer donor dye and the dye on the second nucleotide polymerase is an energy transfer acceptor dye, or wherein the dye on the fluorescently labeled anchor binding molecules and the fluorescently labeled nucleotide analogues is an energy transfer acceptor dye and the dye on the second nucleotide polymerase is an energy transfer donor dye.

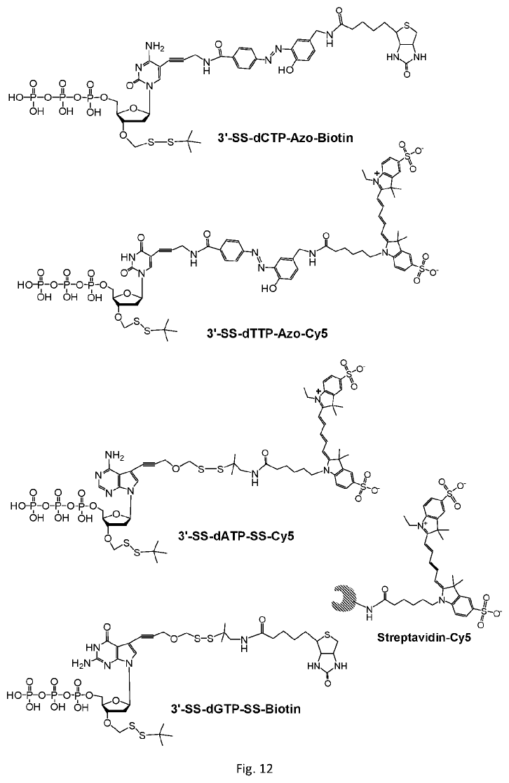

In an embodiment of the instant method, the anchor labeled nucleotide analogues and corresponding anchor binding molecules are those of FIG. 8. In an embodiment of the instant method, the anchor labeled nucleotide analogues and corresponding anchor binding molecules are those of FIG. 12.

The invention further provides a kit comprising all the required nucleotide analogues, polymerases, labeled anchor binding molecules, cleavage agents and other reaction buffer components for carrying out the instant method.

The invention further provides a method of sequencing nucleic acid comprising:
a) providing at least one nucleic acid template hybridized to a primer and a nucleic acid polymerase;
b) providing a first nucleic acid polymerase and extending the primer hybridized to said at least one nucleic acid template with the first nucleic acid polymerase and a fluorescently labeled nucleotide analogue if the nucleotide analogue is complementary to a nucleotide residue which is immediately 5′ to the nucleotide residue of the nucleic acid template hybridized to the 3′ terminal nucleotide residue of the primer, wherein the nucleotide analogue is either:
   (i) a fluorescently labeled nucleotide analogue comprising a base and a fluorescent label and a blocking group attached to the base of the nucleotide analogue via a cleavable linker, wherein said blocking group prevents or greatly reduces incorporation of a subsequent nucleotide analogue into the extended primer strand, and wherein said fluorescent label comprises an energy transfer acceptor or donor dye, and at the same time or immediately afterward providing four unincorporable nucleotide analogues comprising a different fluorescent dye attached to the nucleotide analogue, wherein the fluorescent dye attached to the unincorporable nucleotide analogues is an energy transfer donor or acceptor dye for the fluorescent dye attached to the fluorescently labeled reversibly blocked nucleotide analogue; or
   (ii) a fluorescently labeled reversibly blocked nucleotide analogue comprising a base and a fluorescent label attached to the nucleotide analogue via a cleavable linker and a blocking group at the 3′-OH position, wherein said blocking group prevents incorporation of a subsequent nucleotide analogue into the extended primer strand, and wherein the fluorescent label comprises an energy transfer acceptor or donor dye and at the same time or immediately afterward providing four unincorporable nucleotide analogues comprising a different fluorescent dye attached to the nucleotide analogue, wherein the fluorescent dye attached to the unincorporable nucleotide analogues is an energy transfer donor or acceptor dye for the fluorescent dye attached to the fluorescently labeled reversibly blocked nucleotide analogue;
c) identifying the fluorescence signal due to incorporation of the fluorescently labeled nucleotide analogue onto the primer;
d) cleaving the dye and the blocking group from any primers extended with the fluorescently labeled nucleotide analogues;
e) optionally extending any unextended primer with a 3′ blocked nucleotide analogue without any base modifications;
f) wherein if no fluorescence signal is detected in step c), iteratively repeating steps b) to e) with a fluorescently labeled nucleotide analogue having a different base until the fluorescently labeled nucleotide analogue is incorporated;
g) repeating steps b) to e) with the second one of the four fluorescently labeled nucleotides described in step b;
h) optionally extending any unextended primer with a 3′ blocked nucleotide analogue without any base modifications if the optional chase step e) was not carried out;
i) iteratively performing steps b) to h) for each nucleotide residue of the nucleic acid template,
thereby obtaining the sequence of the nucleic acid template.

In an embodiment of the instant method, the labeled nucleotide analogues provided in step b) are those from step b) (i). In an embodiment of the instant method, the labeled nucleotide analogues provided in step b) are those from step b) (ii). In an embodiment, the dye on the fluorescently labeled nucleotide analogues is Cy5 or ATTO647N and the dye on the unincorporable nucleotide analogues is Cy3. In an embodiment, the cleavable linker is DTM and the cleavage is carried out with THP. In an embodiment, the dyes are dye clusters.

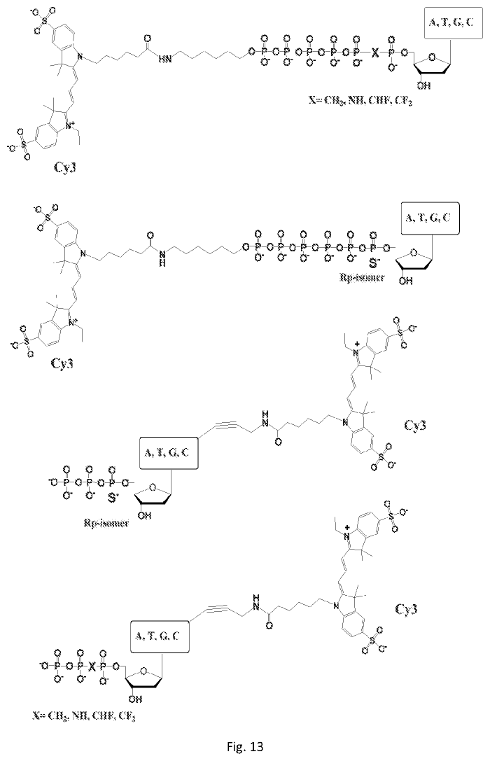

In an embodiment of the instant method, the dye on the fluorescently labeled nucleotide analogues is an energy transfer donor dye and the dye on the unincorporable nucleotide analogues is an energy transfer acceptor dye, or wherein the dye on the fluorescently labeled nucleotide analogues is an energy transfer acceptor dye and the dye on the unincorporable nucleotide analogues is an energy transfer donor dye. In an embodiment, the fluorescently labeled nucleotide analogues are those from FIG. 4 and the unincorporable nucleotide analogues are those from FIG. 13.

The invention also provides kit comprising all the required nucleotide analogues, polymerases, cleavage agents and other reaction buffer components for carrying out the instant method.

The invention also provides a method of sequencing nucleic acid comprising:
a) providing at least one nucleic acid template hybridized to a primer;
b) providing a nucleic acid polymerase and four different labeled nucleotide analogues (A, C, T, G) and extending the primer hybridized to said at least one nucleic acid template with the nucleic acid polymerase and one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) fluorescently labeled nucleotide analogues comprising a base and a blocking group linked to the base via a cleavable linker and a fluorescent label linked distal to the blocking group via either an uncleavable or a different cleavable linker, wherein said blocking group prevents or greatly reduces incorporation of a subsequent nucleotide analogue into the extended primer strand, wherein each of the different nucleotide analogues (A, C, G, T) have the same fluorescent label and different cleavable linkers, and wherein said fluorescent label comprises an energy transfer acceptor or donor dye; or
   (ii) fluorescently labeled nucleotide analogues comprising a base and a fluorescent label attached to the base via a cleavable linker and a blocking group at the 3′-OH position, wherein said blocking group prevents incorporation of a subsequent nucleotide analogue into the extended primer strand, and wherein each of the different nucleotide analogues (A, C, G, T) have the same fluorescent label and different cleavable linkers, and wherein said fluorescent label comprises an energy transfer acceptor or donor dye;
c) at the same time as step b) or immediately afterward, providing four different fluorescently labeled unincorporable nucleotide analogues (A, C, T, G), wherein the fluorescent label is an energy transfer donor or acceptor dye for the energy transfer donor or acceptor dye attached to the fluorescently labeled nucleotides of step b);
d) identifying the fluorescence signal due to incorporation of fluorescently labeled nucleotides or nucleotide analogues;
e) cleaving the dye from the fluorescently labeled nucleotides with a cleaving agent that specifically cleaves one of the linkers but does not cleave any remaining linkers;
f) repeating step c) and identifying any loss of fluorescence due to the cleavage carried out in step e) to partially identify the incorporated nucleotide;
g) iteratively repeating steps e) and f) with cleavable agents that specifically cleave any remaining linkers one-by-one;
h) determining the specific nucleotide analogue incorporated in step b) by comparing the results obtained in multiple iterations of steps f) and i);
i) optionally carrying out a chase step with 3′ blocked nucleotides without any base modifications to extend any remaining primers;
j) cleaving the blocking group and at the same time cleaving any remaining fluorescent labels from the extended primers; and
iteratively carrying out steps b) to j) to obtain the sequence of the nucleic acid template.

In an embodiment of the instant method, the labeled nucleotide analogues provided in step b) are those from step b) (i). In an embodiment of the instant method, the labeled nucleotide analogues provided in step b) are those from step b) (ii). In an embodiment, the dyes are dye clusters.

In an embodiment of the instant method, the reversibly blocked fluorescently labeled nucleotide analogues comprise the same cleavable linker between the base and the blocking group and three of the four analogues comprises a different cleavable linker distal to the blocking group linked to the fluorescent label and the remaining analogue has an uncleavable linker between the blocking group and label, and wherein the last cleavage reaction performed in each cycle cleaves the linker between the base and the blocking group. In an embodiment, the cleavable linkers comprise DTM, azo, allyl and 2-nitrobenzyl and the cleaving agents comprise THP, sodium dithionite, Pd(0) and UV light (˜340 nm) respectively.

In an embodiment of the instant method, the four reversibly blocked fluorescently labeled nucleotide analogues each comprise a different cleavable linker between the base and the fluorescent label, and wherein one of the four cleavage reactions is also capable of removing the 3′ blocking group, and wherein that is the last cleavage reaction performed in each cycle. In an embodiment, the cleavable linkers comprise DTM, azo, allyl and 2-nitrobenzyl and the cleaving agents comprise THP, sodium dithionite, Pd(O) and UV light (˜340 nm) respectively.

In an embodiment of the instant method, the dye on the fluorescently labeled nucleotide analogues is an energy transfer donor dye and the dye on the unincorporable nucleotide analogues is an energy transfer acceptor dye, or wherein the dye on the fluorescently labeled nucleotide analogues is an energy transfer acceptor dye and the dye on the unincorporable nucleotide analogues is an energy transfer donor dye.

In an embodiment of the instant method, the fluorescently labeled nucleotide analogues are those from FIG. 5 and the unincorporable nucleotide analogues are those from FIG. 13. In an embodiment of the instant method, the fluorescently labeled nucleotide analogues are those from FIG. 9 and the unincorporable nucleotide analogues are those from FIG. 13.

The invention further provides a kit comprising all the required nucleotide analogues, polymerases, cleavage agents and other reaction buffer components for carrying out the instant method.

The invention further provides a method of sequencing nucleic acid comprising:
a) a providing at least one nucleic acid template hybridized to a primer;
b) providing a nucleic acid polymerase and four different anchor labeled nucleotide analogues (A, C, T, G) and extending the primer hybridized to said at least one nucleic acid template with the nucleic acid polymerase and one of the anchor labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different anchor labeled nucleotide analogues are either:
   (i) anchor labeled nucleotide analogues each comprising a base, a blocking group linked to the base via a cleavable linker, and an anchor linked to the base via an uncleavable linker distal to the blocking group, wherein said blocking group prevents or greatly reduces incorporation of a subsequent nucleotide analogue into the extended primer strand, wherein each different anchor labeled nucleotide analogue (A, C, G, T) has a different anchor and the same cleavable linker; or
   (ii) anchor labeled nucleotide analogues each comprising a base, an anchor linked to the base via a cleavable linker, and a blocking group at the 3′-OH position, wherein said blocking group prevents incorporation of a subsequent nucleotide analogue into the extended primer strand, and wherein each different anchor labeled nucleotide analogue (A, C, G, T) has different anchor from the remaining anchor labeled nucleotide analogues and the same linker;
c) at the same time as step b) or immediately afterward, providing four different anchor labeled unincorporable nucleotide analogues, wherein the fluorescent label is an energy transfer donor or acceptor dye for the energy transfer donor or acceptor dye attached to the fluorescently labeled nucleotide analogues of step b);
d) identifying the fluorescence signal due to incorporation of fluorescently labeled nucleotide analogues;
e) labeling anchor attached primer extension products with fluorescently labeled anchor binding molecules, wherein the fluorescent label is the same as that on directly labeled nucleotides or nucleotide analogues and wherein the anchor binding molecule binds to the anchor of a specific nucleotide analogue of step b);
f) repeating step c) and identifying newly generated fluorescence signals to partially identify the incorporated nucleotides due to the labeling carried out in step e);
g) repeating steps e) and f) with the fluorescently labeled anchor binding molecule specific for each of the remaining anchors one by one, wherein the same fluorescent dye is attached to all four anchor binding molecules;
h) determining the specific nucleotide analogue incorporated by comparing the results obtained in multiple iterations of steps f) and g);
i) optionally carrying out a chase step with 3′ blocked nucleotides without any base modifications to extend remaining primers;
j) cleaving the blocking group and at the same time cleaving any remaining anchors and fluorescent labels from the extended primers; and and iteratively carrying out steps b) to j) to obtain the sequence of the nucleic acid template.

In an embodiment of the instant method, the four anchor labeled nucleotide analogues are those from step b) i). In an embodiment of the instant method, the four anchor labeled nucleotide analogues are those from step b) ii). In an embodiment, the dyes each comprise dye clusters.

In an embodiment of the instant method, the four anchor labeled nucleotide analogues of step b) (i) each comprise a blocking group attached to the base via the same cleavable linker, a different anchor attached distal to the blocking group via an uncleavable linker, and wherein the anchor of each anchor labeled nucleotide analogue binds to a different anchor binding molecule. In an embodiment, each of the four different anchors independently comprises one of biotin, TCO, DBCO or tetrazine and each of the fluorescently labeled anchor binding molecules independently comprises one of streptavidin, tetrazine, azido and TCO respectively.

In an embodiment of the instant method, the four anchor labeled nucleotide analogues of step b) ii) each comprise a different anchor attached to the base via the same cleavable linker, and wherein the anchor of each anchor labeled nucleotide analogue binds to a different anchor binding molecule. In an embodiment, each of the four different anchors independently comprises one of biotin, TCO, DBCO and tetrazine, and wherein each of the fluorescently labeled anchor binding molecules independently comprises one of streptavidin, tetrazine, azido and TCO respectively.

In an embodiment of the instant method, the dye on the fluorescently labeled anchor binding molecules is an energy transfer donor dye and the dye on the unincorporable nucleotide analogues is an energy transfer acceptor dye, or wherein the dye on the fluorescently labeled anchor binding molecules is an energy transfer acceptor dye and the dye on the unincorporable nucleotide analogues is an energy transfer donor dye.

In an embodiment of the instant method, the anchor labeled nucleotide analogues and corresponding anchor binding molecules are those of FIG. 6. In an embodiment of the instant method, the anchor labeled nucleotide analogues and corresponding anchor binding molecules are those of FIG. 10.

The invention further provides a kit comprising all the required nucleotide analogues, polymerases, labeled anchor binding molecules, cleavage agents and other reaction buffer components for carrying out the instant method.

The invention further provides a method of sequencing nucleic acid comprising:
a) providing at least one nucleic acid template hybridized to a primer;
b) providing a nucleic acid polymerase and four different anchor labeled nucleotide analogues (A, C, T, G) and extending the primer hybridized to said at least one nucleic acid template with the nucleic acid polymerase and one of the anchor labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different anchor labeled nucleotide analogues are either:
   (i) anchor labeled nucleotide analogues each comprising a base and a blocking group linked to the base via the same cleavable linker, wherein said blocking group prevents or greatly reduces incorporation of a subsequent nucleotide analogue into the extended primer strand, wherein two of the different anchor labeled nucleotide analogues (A, C, G, T) comprise the same anchor and the remaining two different anchor labeled nucleotide analogues comprise the same anchor, wherein the anchor of two of the anchor labeled nucleotide analogues is attached distal to the blocking group via an uncleavable linker and the anchor of each of two of the anchor labeled nucleotide analogues is attached distal to the blocking group via the same cleavable linker; or
   (ii) anchor labeled nucleotide analogues each comprising a base, an anchor attached to the base via a cleavable linker, and a blocking group at the 3′-OH position, wherein said blocking group prevents incorporation of a subsequent nucleotide analogue into the extended primer strand, wherein two of the different anchor labeled nucleotide analogues (A, C, G, T) comprise the same anchor and the remaining two different anchor labeled nucleotide analogues comprise the same anchor, wherein the cleavable linker of two of the anchor labeled nucleotide analogues is the same, and wherein the cleavable linker of the remaining two anchor labeled nucleotide analogues is the same and different cleavable groups;
c) at the same time as step b) or immediately afterward, adding all four fluorescently labeled unincorporable nucleotides or nucleotide analogues, wherein the fluorescent label is an energy transfer donor dye for the energy transfer acceptor dye attached to the fluorescently labeled nucleotides of step b);
d) identifying the fluorescence signal due to incorporation of fluorescently labeled nucleotides or nucleotide analogues;
e) labeling anchor attached primer extension products with a fluorescently labeled anchor binding molecule specific for one of the anchors, wherein the fluorescent label is the same as on all anchor binding molecules;
f) repeating step c) and identifying newly generated fluorescence signals to partially or completely identify the incorporated nucleotides due to the labeling carried out in step d);
g) repeating steps e) and f) with a second fluorescently labeled anchor binding molecule specific for a second anchor;
h) cleaving the dye from the fluorescently labeled nucleotides with a specific cleavable agent that cleaves one of the linkers but does not cleave any remaining linkers;
i) repeating step c) and identifying loss of fluorescence due to the cleavage carried out in step h);
j) determining the specific nucleotide analogue incorporated by comparing the results obtained in steps f) and i);
k) optionally carrying out a chase step with 3′ blocked nucleotides without any base modifications to extend remaining primers;
l) cleaving the blocking group and at the same time cleaving any remaining anchors and fluorescent labels from the extended primers; and
iteratively carrying out steps b) to 1) to obtain the sequence of the nucleic acid template.

In an embodiment of the instant method, the four anchor labeled nucleotide analogues are those from step b) i). In an embodiment of the instant method, the four anchor labeled nucleotide analogues are those from step b) ii). In an embodiment, the dyes comprise dye clusters.

In an embodiment of the instant method, the four anchor labeled nucleotide analogues (A, C, G, T) comprise two different cleavable linkers and two different anchors (Anchor 1 and Anchor 2), one cleavable linker (Cleavable Linker 1) between the blocking group and the base of all four of the anchor labeled nucleotide analogues, and a different cleavable linker (Cleavable Linker 2) distal to the blocking group linking the anchor to the blocking group of two of the nucleotide analogues, thereby producing a set of the 4 nucleotides or nucleotide analogues, one consisting of Cleavable Linker 1 with Anchor 1, one consisting of Cleavable Linker 1 with Anchor 2, one consisting of Cleavable Linker 1, Cleavable Linker 2, and Anchor 1, and the last consisting of Cleavable Linker 1, Cleavable Linker 2, and Anchor 2, wherein cleavage of Cleavable Linker 2 and detection of fluorescence signals is carried out prior to the final cleavage of Cleavable Linker 1. In an embodiment, cleavable linkers comprise DTM and azo, the cleaving agents comprise THP and sodium dithionite respectively, the anchors comprise biotin and TCO, and the fluorescently labeled anchor binding molecules comprise streptavidin and tetrazine respectively.

In an embodiment of the instant method, the four different anchor labeled nucleotide analogues (A, C, G, T) comprise 2 different cleavable linkers and 2 different anchors (Anchor 1 and Anchor 2), wherein one cleavable linker (Cleavable Linker 1) links the base of a nucleotide analogue to an anchor, the other cleavable linker (Cleavable Linker 2) links the base of a nucleotide analogue to an anchor, wherein one of the anchor labeled nucleotide analogues has Cleavable Linker 1 and Anchor 1, another has Cleavable Linker 1 and Anchor 2, another has Cleavable Linker 2 and Anchor 1, and the final has Cleavable Linker 2 and Anchor 2, thereby producing a set of 4 nucleotide analogues, wherein an agent capable of cleaving Cleavable Linker 1 also removes the 3′-OH blocking group, and wherein cleavage of Cleavable Linker 2 and determination of signal is performed prior to cleavage of Cleavable Linker 1. In an embodiment, the cleavable linkers independently comprise DTM or azo, the cleaving agents comprise THP and sodium dithionite respectively, the anchors comprise biotin and TCO, and the fluorescently labeled anchor binding molecules comprise streptavidin and tetrazine respectively.

In an embodiment of the instant method, the dye on the fluorescently labeled anchor binding molecules is an energy transfer donor dye and the dye on the unincorporable nucleotide analogues is an energy transfer acceptor dye, or wherein the dye on the fluorescently labeled anchor binding molecules is an energy transfer acceptor dye and the dye on the unincorporable nucleotide analogues is an energy transfer donor dye.

In an embodiment of the instant method, the anchor labeled nucleotide analogues and corresponding anchor binding molecules are those of FIG. 7. In an embodiment of the instant method, the anchor labeled nucleotide analogues and corresponding anchor binding molecules are those of FIG. 11.

The invention further provides a kit comprising all the required nucleotide analogues, polymerases, labeled anchor binding molecules, cleavage agents and other reaction buffer components for carrying out the instant method.

A method of sequencing nucleic acid comprising:
a) providing at least one nucleic acid template hybridized to a primer;
b) providing a nucleic acid polymerase, four different labeled nucleotide analogues (A, C, T, G) and extending the primer hybridized to said at least one nucleic acid template with the nucleic acid polymerase and one of the nucleotide analogues if the nucleotide analogue is complementary to a nucleotide residue which is immediately 5′ to the nucleotide residue of the nucleic acid template hybridized to the 3′ terminal nucleotide residue of the primer, wherein the four labeled nucleotide analogues are either:
   (i) two different fluorescently labeled nucleotide analogues that comprise a base and a blocking group linked to the base via
   a first cleavable linker,
      wherein one of the fluorescently labeled nucleotide analogues comprises a fluorescent label attached distal to the blocking group via a second cleavable linker wherein said fluorescent label is an energy transfer donor or acceptor dye, and
      wherein the remaining fluorescently labeled nucleotide analogue comprises a fluorescent label attached distal to the blocking group via an uncleavable linker wherein said fluorescent label is an energy transfer donor or acceptor dye, and
   two different anchor labeled nucleotide analogues that comprise a base and a blocking group linked to the base via the first cleavable linker,
      wherein one of the anchor labeled nucleotide analogues comprises an anchor attached distal to the blocking group via the second cleavable linker, and
      wherein one the remaining anchor labeled nucleotide analogue comprises the same anchor attached distal to the blocking group via an uncleavable linker; or
   (ii) two different fluorescently labeled nucleotide analogues that comprise a base and a blocking group at the 3′-OH position,
      wherein one of the fluorescently labeled nucleotide analogues comprises a fluorescent label attached to the base via a first cleavable linker wherein said fluorescent label is an energy transfer donor or acceptor dye, and
      wherein the remaining fluorescently labeled nucleotide analogue comprises a fluorescent label attached to the base via a second cleavable linker wherein said fluorescent label is an energy transfer donor or acceptor dye, and
   two different anchor labeled nucleotide analogues that comprise a base and a blocking group at the 3′-OH position,
   wherein one of the anchor labeled nucleotide analogues comprises an anchor linked to the base via the first cleavable linker, and
   wherein the remaining anchor labeled nucleotide analogue comprises the same anchor linked to the base via a second cleavable linker;
c) at the same time as step b or immediately afterward, adding all four fluorescently labeled unincorporable nucleotides or nucleotide analogues, wherein the fluorescent label is an energy transfer donor or acceptor dye for the energy transfer acceptor donor or acceptor dye attached to the fluorescently labeled nucleotides of step b);
d) after a chase step with 3′ blocked nucleotides without any base modifications to extend remaining primers, identifying the fluorescence resonance energy transfer (FRET) signal due to incorporation of any fluorescently labeled nucleotide analogues;
e) labeling anchor attached primer extension products with a fluorescently labeled anchor binding molecule specific for one of the anchors, wherein the fluorescent label is the same as that on the fluorescently labeled nucleotide analogues;
f) repeating step c) and identifying any newly generated FRET signals to partially identify the incorporated nucleotides due to the labeling carried out in step e);
g) cleaving the dye from the fluorescently labeled nucleotides with a specific cleavable agent that cleaves one of the linkers but does not cleave the orthogonal linker;
h) repeating step c) and identifying any loss of FRET signals due to the cleavage carried out in step g) to completely identify the incorporated nucleotide;
i) determining the specific nucleotide analogue incorporated by comparing the results obtained in steps f) and h);
j) cleaving the blocking group and at the same time cleaving any remaining anchors and fluorescent labels from the extended primers; and
iteratively carrying out steps b) to j) to obtain the sequence of the nucleic acid template.

In an embodiment of the instant method, the four anchor labeled nucleotide analogues are those from step b) i). In an embodiment of the instant method, the four anchor labeled nucleotide analogues are those from step b) ii). In an embodiment, the dyes comprise dye clusters.

In an embodiment of the instant method, the four labeled nucleotide analogues comprise 2 different cleavable linkers and 2 different anchors, one cleavable linker (Cleavable Linker 1) between the blocking group and all four of the nucleotides or nucleotide analogues, and a different cleavable linker (Cleavable Linker 2) between the base and the anchors but distal to the blocking group for two of the nucleotides or nucleotide analogues, thereby producing a set of the 4 nucleotides or nucleotide analogues, one comprising Cleavable Linker 1 and Anchor 1, one comprising Cleavable Linker 1 with Anchor 2, one comprising Cleavable Linker 1, Cleavable Linker 2, and Anchor 1, and the last comprising Cleavable Linker 1, Cleavable Linker 2, and Anchor 2, wherein cleavage of Cleavable Linker 2 and detection of fluorescence signals is carried out prior to the final cleavage of Cleavable Linker 1. In an embodiment, Cleavable Linker 1 comprises DTM, Cleavable Linker 2 comprises azo, the cleaving agents comprise THP and sodium dithionite respectively, the anchors comprise biotin (Anchor 1) and TCO (Anchor 2), and the fluorescently labeled anchor binding molecules comprise streptavidin and tetrazine respectively.

In an embodiment of the instant method, the four labeled nucleotide analogues 2 different cleavable linkers and 2 different anchors, the first type of cleavable linker between one of the nucleotides or nucleotide analogues and one of the anchors, the first type of cleavable linker between the second nucleotide analogue and the second type of anchor, a second type of cleavable linker between the third nucleotide analogue and the first anchor, and the second type of cleavable linker between the fourth nucleotide analogue and the second anchor, thereby producing a set of 4 nucleotides or nucleotide analogues, wherein cleavage of the first type of cleavable linker is also capable of removing the 3′-OH blocking group, and wherein cleavage of the second type of cleavable linker and determination of signal is performed prior to cleavage of the first type of cleavable linker. In an embodiment, the cleavable linkers comprise DTM and azo, the cleaving agents comprise THP and sodium dithionite respectively, the anchors comprise biotin and TCO, and the fluorescently labeled anchor binding molecules comprise streptavidin and tetrazine respectively.

In an embodiment of the instant method, the dye on the fluorescently labeled anchor binding molecules and fluorescently labeled nucleotide analogues is an energy transfer donor dye and the dye on the unincorporable nucleotide analogues is an energy transfer acceptor dye, or wherein the dye on the fluorescently labeled anchor binding molecules and the fluorescently labeled nucleotide analogues is an energy transfer acceptor dye and the dye on the unincorporable nucleotide analogues is an energy transfer donor dye.

In an embodiment of the instant method, the anchor labeled nucleotide analogues and corresponding anchor binding molecules are those of FIG. 8.

In an embodiment of the instant method, the anchor labeled nucleotide analogues and corresponding anchor binding molecules are those of FIG. 12.

The invention further provides a kit comprising all the required nucleotide analogues, polymerases, labeled anchor binding molecules, cleavage agents and other reaction buffer components for carrying out the instant method.

### TERMS

As used herein, and unless stated otherwise, each of the following terms shall have the definition set forth below.
A—Adenine;
C—Cytosine;
G—Guanine;
T—Thymine;
U—Uracil;
DNA—Deoxyribonucleic acid;
RNA—Ribonucleic acid;

“Nucleic acid” shall mean, unless otherwise specified, any nucleic acid molecule, including, without limitation, DNA, RNA and hybrids thereof. In an embodiment the nucleic acid bases that form nucleic acid molecules can be the bases A, C, G, T and U, as well as derivatives thereof.

“Derivatives” or “analogues” of these bases are well known in the art, and are exemplified in PCR Systems, Reagents and Consumables (Perkin Elmer Catalogue 1996-1997, Roche Molecular Systems, Inc., Branchburg, N.J., USA).

A “nucleotide residue” is a single nucleotide in the state it exists after being incorporated into, and thereby becoming a monomer of, a polynucleotide. Thus, a nucleotide residue is a nucleotide monomer of a polynucleotide, e.g. DNA, which is bound to an adjacent nucleotide monomer of the polynucleotide through a phosphodiester bond at the 3′ position of its sugar and is bound to a second adjacent nucleotide monomer through its phosphate group, with the exceptions that (i) a 3′ terminal nucleotide residue is only bound to one adjacent nucleotide monomer of the polynucleotide by a phosphodiester bond from its phosphate group, and (ii) a 5′ terminal nucleotide residue is only bound to one adjacent nucleotide monomer of the polynucleotide by a phosphodiester bond from the 3′ position of its sugar.

“Substrate” or “Surface” shall mean any suitable medium present in the solid phase to which a nucleic acid or an agent may be affixed. Non-limiting examples include chips, beads, nanopore structures and columns. In an embodiment the solid substrate can be present in a solution, including an aqueous solution, a gel, or a fluid.

“Hybridize” shall mean the annealing of one single-stranded nucleic acid to another nucleic acid based on the well-understood principle of sequence complementarity. In an embodiment the other nucleic acid is a single-stranded nucleic acid. The propensity for hybridization between nucleic acids depends on the temperature and ionic strength of their milieu, the length of the nucleic acids and the degree of complementarity. The effect of these parameters on hybridization is well known in the art (see Sambrook J, Fritsch E F, Maniatis T. 1989. Molecular cloning: a laboratory manual. Cold Spring Harbor Laboratory Press, New York). As used herein, hybridization of a primer sequence, or of a DNA extension product, to another nucleic acid shall mean annealing sufficient such that the primer, or DNA extension product, respectively, is extendable by creation of a phosphodiester bond with an available nucleotide or nucleotide analog capable of forming a phosphodiester bond.

As used herein, unless otherwise specified, a base which is “unique” or “different from” another base or a recited list of bases shall mean that the base has a different structure from the other base or bases. For example, a base that is “unique” or “different from” adenine, thymine, and cytosine would include a base that is guanine or a base that is uracil.

As used herein, unless otherwise specified, a label or tag moiety which is “different” from the label or tag moiety of a referenced molecule means that the label or tag moiety has a different chemical structure from the chemical structure of the other/referenced label or tag moiety.

As used herein, unless otherwise specified, “primer” means an oligonucleotide that upon forming a duplex with a polynucleotide template, is capable of acting as a point of polymerase incorporation and extension from its 3′ end along the template, thereby resulting in an extended duplex.

As used herein, “alkyl” includes both branched and straight-chain saturated aliphatic hydrocarbon groups having the specified number of carbon atoms and may be unsubstituted or substituted. Thus, C1-Cn as in “C1-Cn alkyl” includes groups having 1, 2, . . . , n−1 or n carbons in a linear or branched arrangement. For example, a “C1-C5 alkyl” includes groups having 1, 2, 3, 4, or 5 carbons in a linear or branched arrangement, and specifically includes methyl, ethyl, n-propyl, isopropyl, n-butyl, t-butyl, and pentyl.

As used herein, “alkenyl” refers to a non-aromatic hydrocarbon group, straight or branched, containing at least 1 carbon to carbon double bond, and up to the maximum possible number of non-aromatic carbon-carbon double bonds may be present, and may be unsubstituted or substituted. For example, “C2-C5 alkenyl” means an alkenyl group having 2, 3, 4, or 5, carbon atoms, and up to 1, 2, 3, or 4, carbon-carbon double bonds respectively. Alkenyl groups include ethenyl, propenyl, and butenyl.

The term “alkynyl” refers to a hydrocarbon group straight or branched, containing at least 1 carbon to carbon triple bond, and up to the maximum possible number of non-aromatic carbon-carbon triple bonds may be present, and may be unsubstituted or substituted. Thus, “C2-C5 alkynyl” means an alkynyl group having 2 or 3 carbon atoms and 1 carbon-carbon triple bond, or having 4 or 5 carbon atoms and up to 2 carbon-carbon triple bonds. Alkynyl groups include ethynyl, propynyl and butynyl.

The term “substituted” refers to a functional group as described above such as an alkyl, or a hydrocarbyl, in which at least one bond to a hydrogen atom contained therein is replaced by a bond to non-hydrogen or non-carbon atom, provided that normal valencies are maintained and that the substitution(s) result(s) in a stable compound. Substituted groups also include groups in which one or more bonds to a carbon(s) or hydrogen(s) atom are replaced by one or more bonds, including double or triple bonds, to a heteroatom. Non-limiting examples of substituents include the functional groups described above, and for example, N, e.g. so as to form —CN.

Throughout this application, most of the nucleotide analogues used in the various schemes contain dithiomethyl (DTM(SS)) blocking groups at the 3′ O position and often contain cleavable DTM(SS) groups in the linkers between the base and the dye or anchor molecules. Previous methods have placed SS groups between the base and dye but after cleavage a free, reactive —SH group is formed which has to be capped with iodoacetamide before the second extension reaction can be carried out (Mitra et al 2003, Turcatti et al 2008). This limits the length of sequencing reads. The new DTM based linker between the base and the fluorophore disclosed in this application does not require capping of the resulting free SH group after cleavage with THP as the cleaved product instantaneously collapses to the stable OH group.

Section I: New SBS Methods Involving Energy Transfer with Donor Fluorophore on Polymerase and Acceptor Fluorophore on Nucleotide Analogues

Ju et al (WO 2017/176677 A1) previously described an energy transfer approach for SBS utilizing donor dye on the polymerase and acceptor dyes on either nucleotide reversible terminators (NRTs), unincorporable nucleotides, or natural nucleotides. This places the donor and acceptor dyes within 2-4 nm, less than the 10 nm needed for efficient fluorescence resonance energy transfer (FRET). Three general types of SBS were therein described. The first used 3′-blocked NRTs, wherein the acceptor dye was at the terminal phosphate position. The polymerase reaction was carried out in the presence of a non-catalytic metal such as Sr⁻⁺ to provide sufficient time for measuring energy transfer from donor to acceptor in the polymerase ternary complex consisting of enzyme, template, primer, and nucleotide. Then a catalytic metal such as Mg⁺⁺ was added to allow incorporation, while at the same time, releasing the pyrophosphate or polyphosphate along with the acceptor dye. Finally, cleavage of the 3′ blocking group restored the 3′-OH for the next cycle.

The second general type of SBS therein disclosed used unincorporable nucleotides bearing acceptor dyes on the base or terminal phosphate position. These nucleotides bind long enough for measurement of FRET in the ternary complex, and are then replaced with unlabeled NRTs in a competition reaction, followed by cleavage of the blocking group on the incorporated NRT. The third general type of SBS therein disclosed used natural nucleotides bearing combinatorial FRET acceptor dyes on the terminal phosphate. A real-time single molecule approach was described.

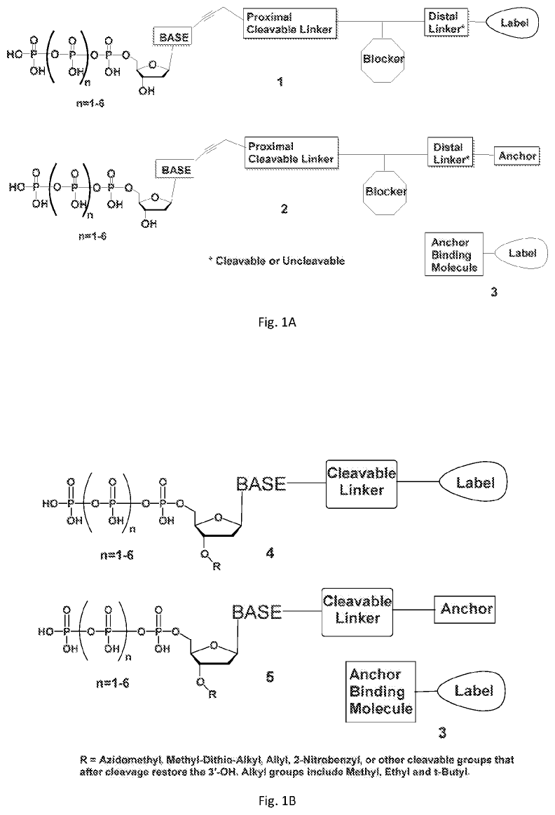

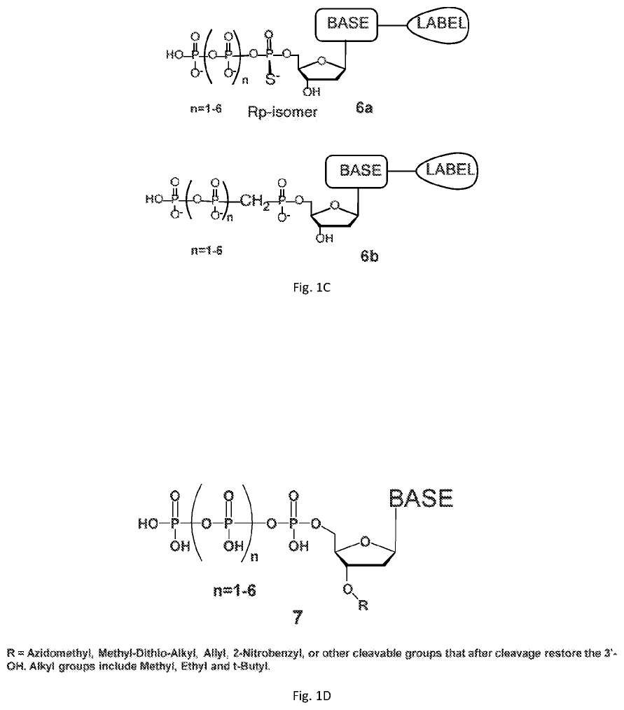

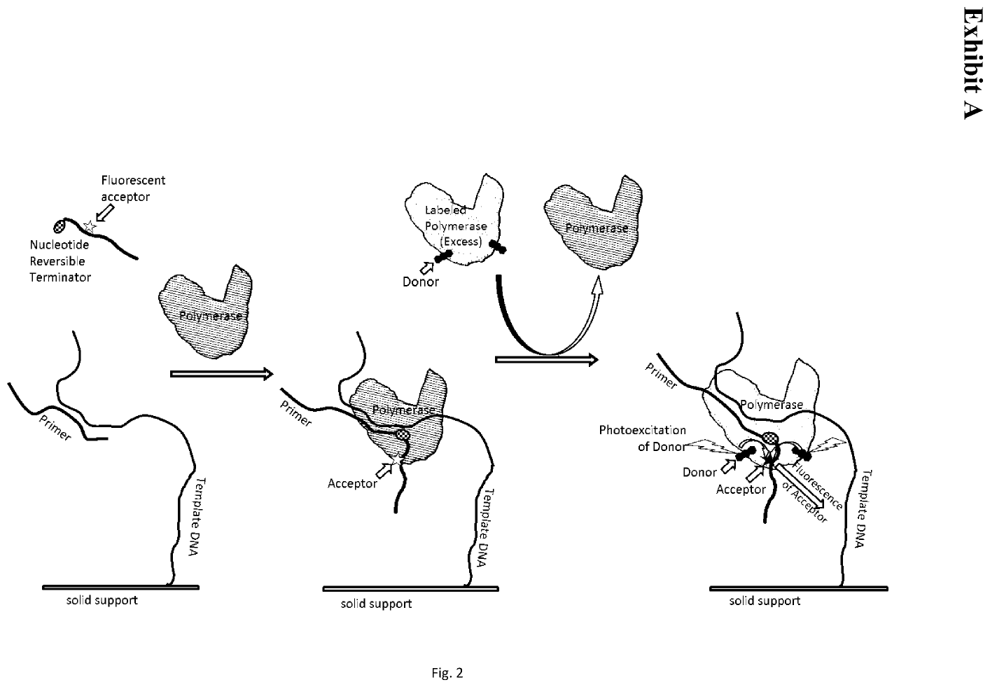

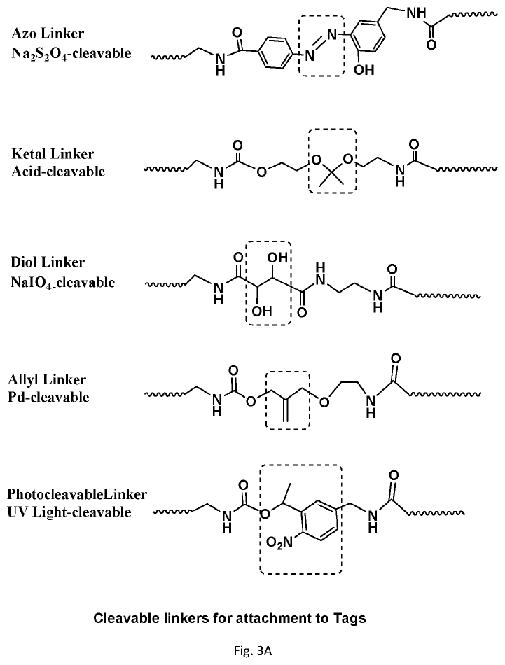

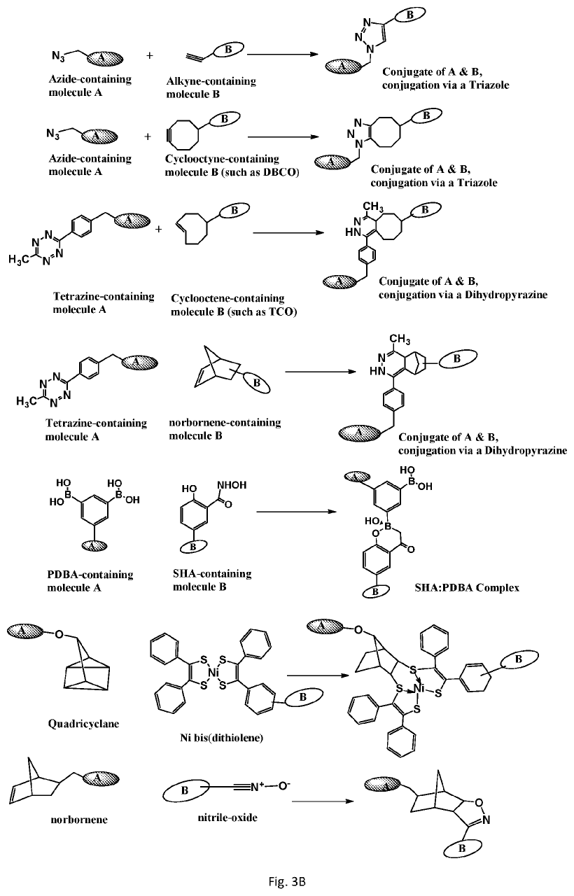

The invention disclosed herein provides a novel and alternative type of SBS (FIG. 1) in which acceptor dye-bearing nucleotide analogues (reversible terminators, including virtual terminators or 3′ blocked NRTs) are incorporated using an unlabeled polymerase, after which the polymerase is replaced with one bearing donor dyes. Extension is carried out with template, primer, reversible terminators attached to an acceptor dye (e.g., Cy5) via a cleavable linker on either the 3′ position or the base, and an unlabeled DNA polymerase that accepts such nucleotides with high efficiency. After washing away unincorporated nucleotides, a polymerase bearing a donor dye (e.g., Cy3 or Cy2) attached to one or more amino acids near the active site is flowed into the system. The labeled polymerase can exchange with the unlabeled polymerase for binding to the template/extended primer. Energy transfer is detected by exciting the donor dye and measuring emission from the acceptor dye. After an additional wash, the dye on the nucleotide is cleaved and the 3′-OH regenerated to allow the next cycle of SBS to occur.

Though attachment of the donor dye molecule(s) to polymerase and acceptor dyes to the nucleotides is the method shown in the examples described herein, the opposite placement is also within the scope of the invention. The approach can be used for either ensemble or single molecule sequencing. Because the polymerase needs to come off and on during this approach, either the template or primer should be attached to the surface, without amplification in the case of single molecule sequencing, or following amplification (e.g., cluster formation) in the case of ensemble sequencing.

Though not shown, the invention provides a simplified variation of the schemes presented herein, the fluorescently labeled polymerase can be used for both the incorporation and the energy transfer, provided that the fluorescently decorated polymerase retains essential aspects of its polymerase activity (high reaction speed and fidelity).

The approach can be used with a variety of types of nucleotide analogues and with a variety of single color SBS approaches. For instance, the acceptor dyes can be attached to either traditional nucleotide reversible terminators (NRTs) with cleavable 3′ blocking groups and cleavable linkers for attachment of the dyes on the base, they can be attached to virtual terminators with 3′-OH groups and cleavable linkers for attaching blocking groups and dyes to the base, or they even can be attached to dideoxynucleotides (ddNTPs) when the latter are used in combination with unlabeled NRTs in a hybrid SBS/Sanger sequencing approach (Ju et al US 2016/0024574 A1; Guo et al 2008). The 3′-blocked NRTs and virtual terminator designs can be used for single molecule or ensemble sequencing. At the current state of the art, the ddNTP design can only be used for ensemble sequencing.

The invention provides, for one-color sequencing by synthesis, NRTs or virtual terminators can be added one at a time (e.g., A, G, C, T, A, G, C, T, etc.) as in Schemes P1 and P2, or they can be added together. The invention also provides, in the case of simultaneous addition, the dyes can be attached via 4 different cleavable linkers with imaging after each cleavage step (Schemes P3 and P7), via 4 anchors and anchor binding molecules with imaging after each labeling step (Schemes P4 and PB), or via orthogonal combinations of 2 cleavable linkers and 2 anchors and anchor binding molecules with imaging after each labeling and the cleavage step (Schemes P5 and P9).

Finally, the invention provides embodiments wherein the acceptor dye may be directly attached to one or more of the nucleotide analogues and indirectly attached via anchors and anchor binding molecules to the other nucleotide analogues with imaging after extension, labeling, and cleavage steps (Schemes P6 and P10).

Though the non-limiting examples of the invention shown herein utilize Cy3 (or Cy2) as the donor and Cy5 as the acceptor fluorophores, a multiplicity of other donor-acceptor pairs are available and can be used. Though all the non-limiting examples shown herein are single color methods to take best advantage of the FRET technique, 2-color and even 4-color schemes can be considered with the use of combinatorial FRET acceptor dyes as part of the provided invention. While the non-limiting examples described herein show measurement of energy transfer by appearance of an emission signal for the acceptor dye, the invention also provides a ratiometric method that tracks loss of donor emission and gain of acceptor emission, which offers additional benefits with regard to accuracy and background characterization, particularly if combinatorial methods are used.

There are many approaches for conjugating the donor dye molecules to amino acids of the polymerase.

People skilled in the art will know of several methods to derivatize the polymerase, taking advantage of key amino acid residues such as lysines (via N-hydroxysuccinimide) or cysteines (via maleimide). These linkages can involve homo- or hetero-bifunctional reagents, two-linker systems such as that available from Trilink (Chromalink reagents), or a Diels-Alder reaction between TCO and tetrazine. The presence of NHS esters or isothiocyanate groups on the end of a linker molecule allows its attachment to the N-terminal amino group or primary amino groups on lysines of the polymerase. The presence of maleimide or iodoacetamide on a linker allows its attachment to the SH group on cysteines of the polymerase. Such groups can also be used to connect to amino- or sulfhydryl-modified dye molecules. Selecting the appropriate ratio of dye to polymerase is determined based on the number of available modifiable amino acids in the latter.

A wide variety of other conjugation strategies are described in the literature, some of which involve placement of unnatural amino acids at desired positions for highly specific attachment of the dye. It is possible to tune colloidal quantum dots (QDs) to serve as energy transfer donors. A method for attaching QDs to an active T7 RNA polymerase molecule has been published (Eriksen et al. 2013).

### Example P1: Single Color SBS with Energy Transfer Between Donor Dye on Polymerase and Acceptor Dye on Nucleotide Analogue and One-by-One Addition of the Four Nucleotide Analogues (Schemes P1 and P2)

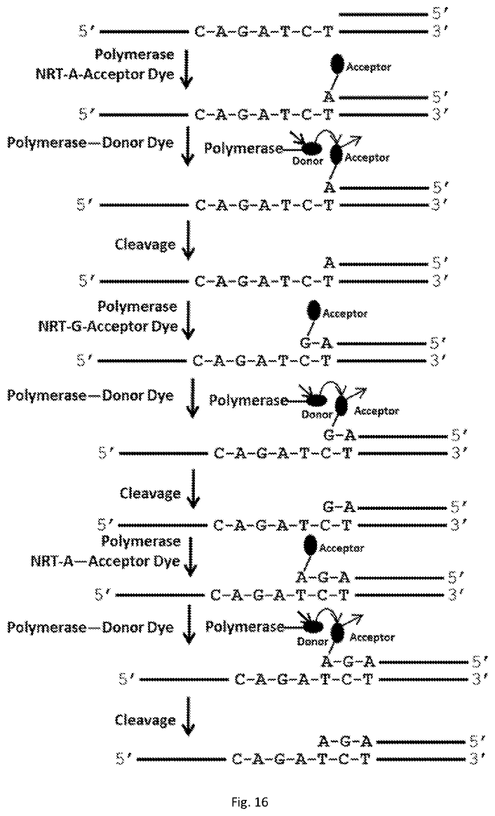

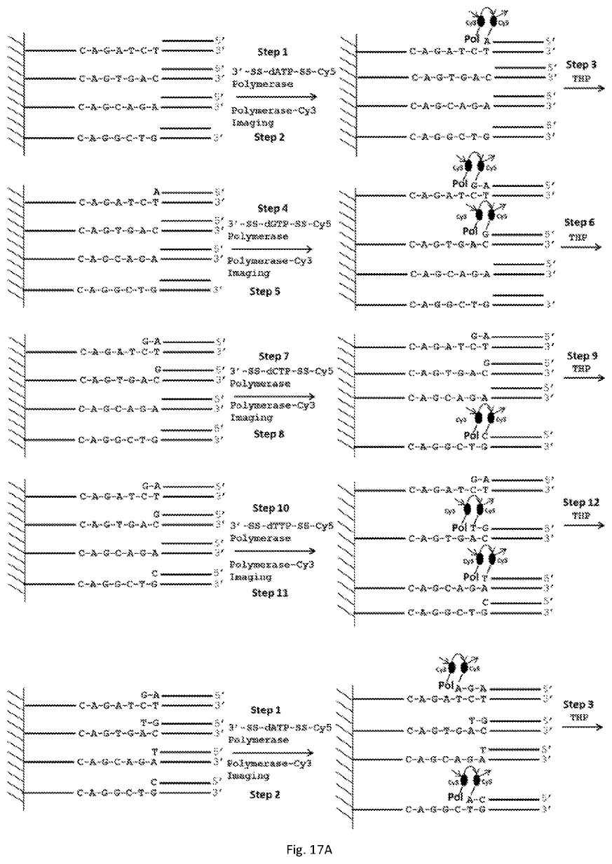

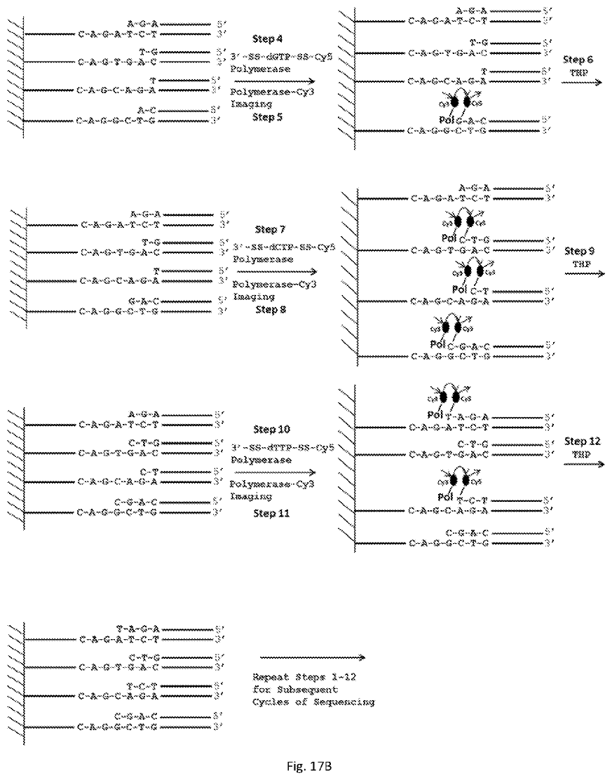

The general scheme presented in Scheme P1 (FIG. 16) demonstrates the basic 1-color method with addition of one nucleotide analogue (virtual terminator or 3′-blocked NRT) at a time. The example of Scheme P1 illustrated in FIG. 16 assumes that the correct nucleotide analogue has been added in each cycle of addition.

In the initial step, the DNA polymerase reaction permits incorporation, at the 3′ end of the growing primer chain, of the nucleotide analogue complementary to the base at the same position on the template DNA; this nucleotide has a FRET acceptor fluorophore attached via a cleavable linker. In the second step, the polymerase is exchanged with a polymerase conjugated to one or more of the FRET donor fluorophores. The donor and acceptor dyes are brought close enough to each other for energy transfer to take place from donor to acceptor when the donor is excited near its absorption maximum. The exact placement of the donor dyes on the polymerase is designed to increase the likelihood of maximal FRET to the acceptor dyes on the reversible terminators. The resulting emission of the acceptor fluorophore (and if desired, of the donor fluorophore) is measured.

These excitation, energy transfer, and emission events are indicated by the arrows in FIG. 16. In the third step, the acceptor dyes and any groups that block further nucleotide incorporation are cleaved. These three steps are repeated in each cycle. Washes are carried out between steps to remove unincorporated NRTs, polymerases, chemical cleavage agents and other reaction components.

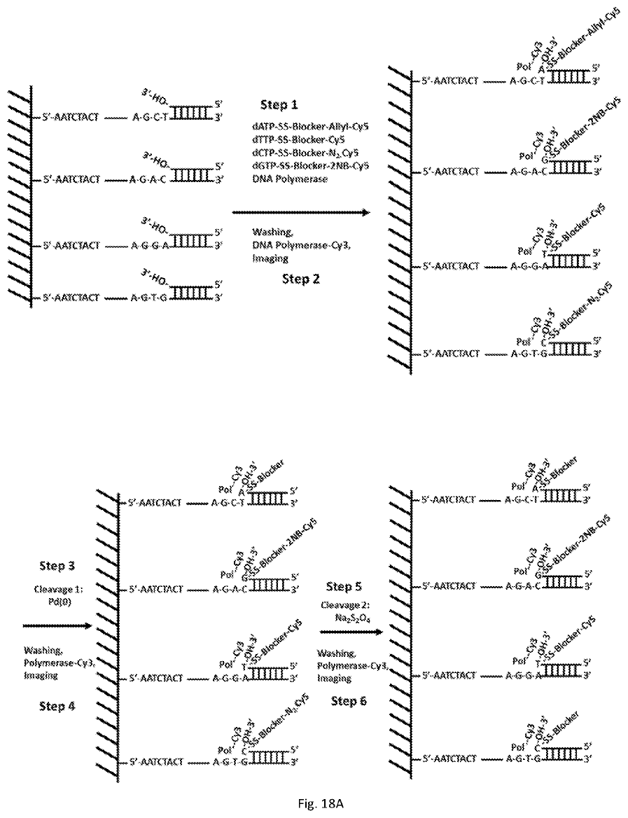

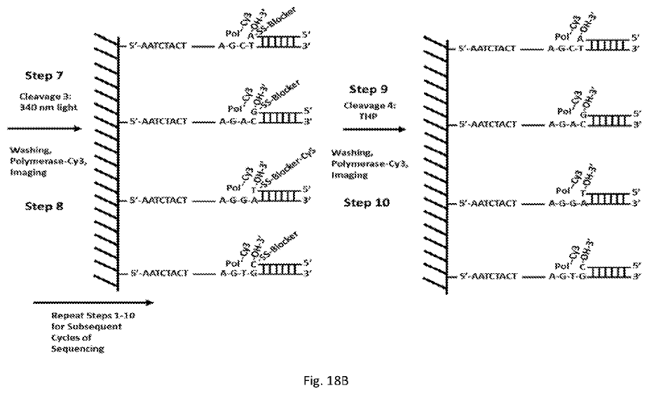

Scheme P2 (FIGS. 17A-17B) is a specific example of Scheme P1, in which multiple cycles are carried out, using Cy3 as donor and Cy5 as acceptor and with addition of NRTs in the order A, G, C, T, A, G, C, T, etc. As with all such methods involving addition of one nucleotide at a time, the progression of SBS will be sequence dependent. Thus, in Scheme P2, the 4 DNA templates are extended at somewhat different rates, at least over the short distances shown. The use of 3′-blocked NRTs (or virtual terminators, if desired) instead of natural nucleotides is necessary to accurately decode homopolymer stretches (e.g., AAA or GG). Example structures of FRET-acceptor dye labeled NRTs for use in this scheme are presented in FIG. 4.

### Example P2: Single Color SBS with Energy Transfer Between Donor Dye on Polymerase and Acceptor Dye on Nucleotide Analogue and Simultaneous Addition of the Four Nucleotide Analogues (Schemes P3-P10)

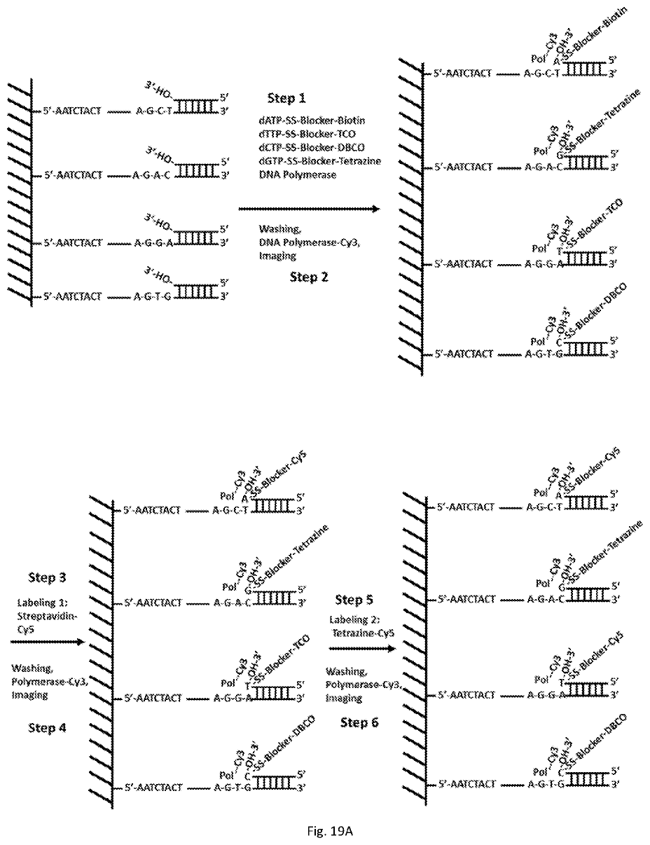

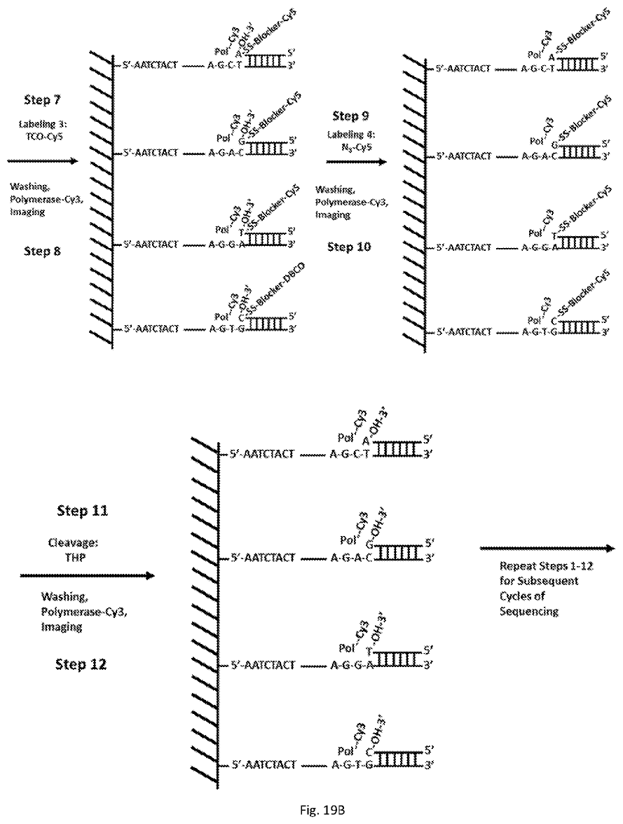

In Scheme P3 (FIGS. 18A-18B), the approach is shown with virtual terminators (nucleotide analogues bearing bulky or acidic groups (blockers) attached to the base via a cleavable linker) that largely prevent incorporation of the subsequent nucleotide. In addition to this blocking group, the FRET acceptor fluorescent dye (Cy5) is also attached via a second linker, distal to the blocker, on the base. In the example scheme depicted in FIGS. 18A-18B, the linkers for the 4 different bases have a DTM(SS) group between the base and the blocking group, and 3 different cleavable groups, Azo (N₂) for C, allyl for A, 2-nitrobenzyl (2-NB) for G, or no additional cleavable group between the blocking group and the dye for T. The four virtual terminators are added together. Structures of these and other cleavable linkers are presented in FIG. 3A.

After incorporation and replacement of the unlabeled polymerase with polymerase containing the donor dye (Cy3 shown, but Cy2 could also be used), FRET is measured, presenting as a non-specific signal due to incorporation of any of the 4 nucleotides. In the following steps, the linkers are cleaved one by one, and re-addition of Cy3-polymerase and imaging is repeated to see if the FRET signal is retained or lost. Loss of the FRET signal immediately reveals the specific nucleotide analogue incorporated. Thus loss of signal after cleavage of the allyl linker with Pd(0) indicates incorporation of A, loss of signal after cleavage of the Azo linker with sodium dithionite indicates incorporation of C, loss of signal after photocleavage of the 2-NB linker with ˜340 nm light indicates incorporation of G, and loss of signal after cleavage of the DTM(SS) linker with THP indicates incorporation of T. The THP cleavage (which should always be the final cleavage reaction in each cycle) also removes all the blocking groups, readying the extended primer for the next cycle of SBS.

Though not indicated in the scheme as illustrated in FIGS. 18A-18B, if ensemble sequencing is performed, a chase step can be carried out with non-fluorescent NRTs once during or after each full cycle to guarantee that all the growing primer chains remain in register, in order to avoid skipping bases during the detection steps. In this case cleavage of the 3′ blocking group, e.g., DTM(SS) or azidomethyl, on these chase nucleotides, is required before commencement of the next cycle.

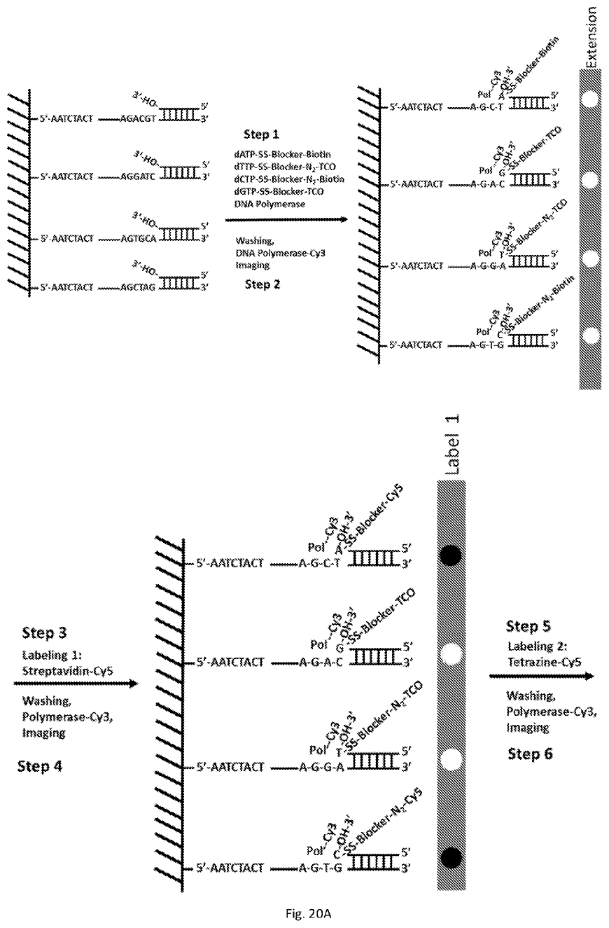

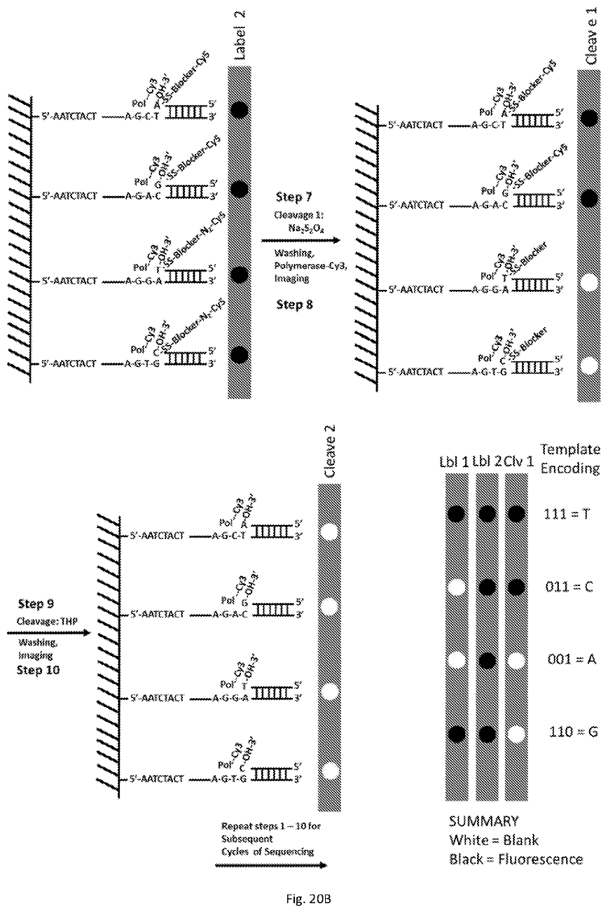

In Scheme P4 (FIGS. 19A-19B), again for virtual terminators, instead of sequential cleavage of the dyes on the four bases in each cycle, the approach involves sequential labeling of the four bases. The blocking groups on the virtual terminators are all connected to the base via the same cleavable linker, and a non-cleavable linker to one of four different anchors is present distal to the blocker. In the example scheme depicted, the linkers for the 4 different bases have a DTM(SS) group between the base and the blocking group, and the anchors for A, C, G and T are biotin, DBCO, Tetrazine and TCO respectively. The four virtual terminators are added together. Structures of these and other anchors and anchor binding molecules are presented in FIG. 3B.

After incorporation and replacement of the unlabeled polymerase with polymerase containing the donor dye (Cy3 shown, but Cy2 could also be used), FRET is measured, presenting as a non-specific signal due to incorporation of any of the 4 nucleotides. In the following steps, dyes attached to anchor-binding molecules are added one by one, and re-addition of Cy3-polymerase and imaging is repeated to see if a new FRET signal appears. Gain of the FRET signal immediately reveals the specific nucleotide analogue incorporated. Thus, gain of FRET signal after labeling with streptavidin-Cy5 indicates extension with A, gain of FRET signal after labeling with N₃-Cy5 indicates extension with C, gain of FRET signal after labeling with TCO-Cy5 indicates extension with G, and gain of FRET signal after labeling with Tetrazine-Cy5 indicates extension with T. Finally, treatment with THP removes all the dyes and blocking groups, readying the extended primer for the next cycle of SBS. With ensemble sequencing, a chase step can be carried out in the same way as described in Scheme P3.

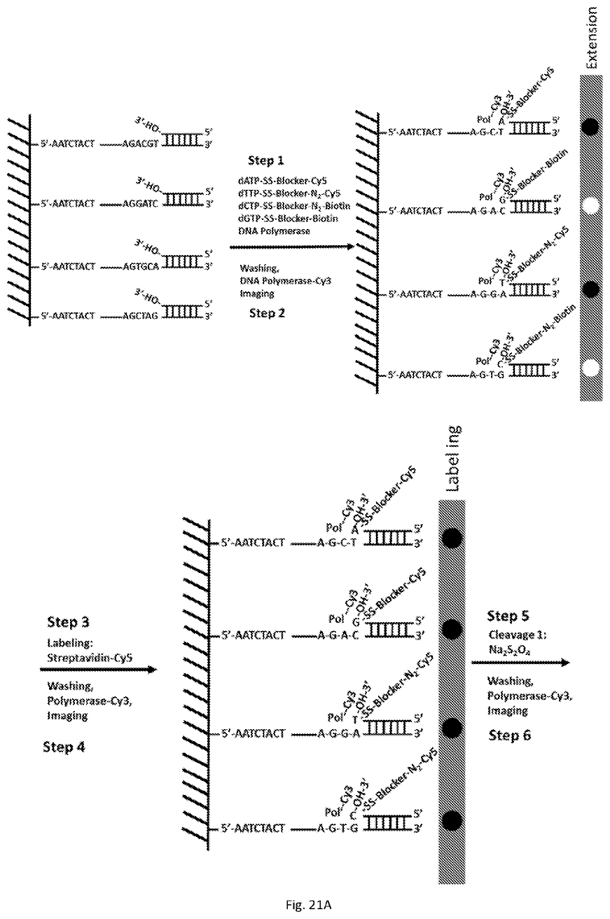

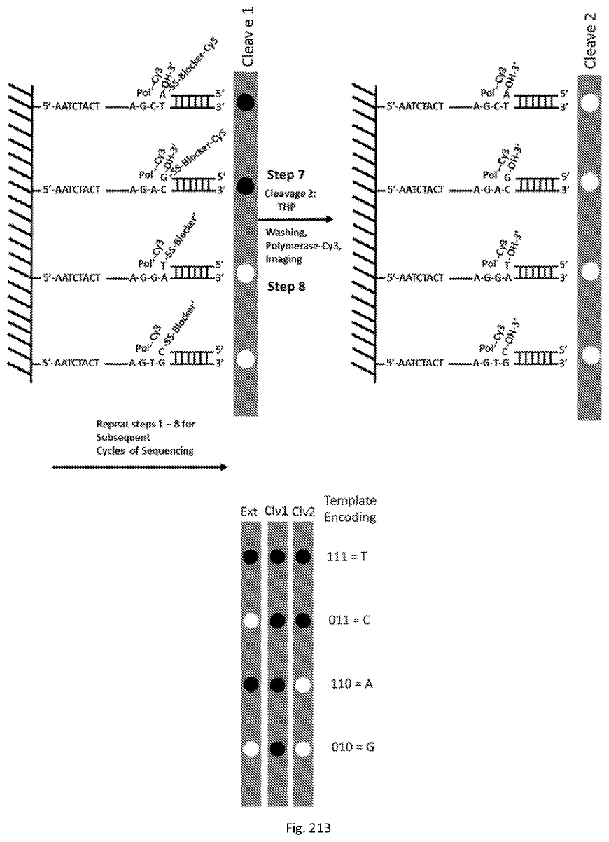

In Scheme P5 (FIGS. 20A-20B), the approach is shown with virtual terminators as in Scheme P3, in this case with a DTM(SS) group between the base and the blocker, and either a DTM(SS) or Azo linker to the anchor molecule (biotin or TCO) distal to the blocking group. Each nucleotide analogue has a different combination of cleavable linker (Azo or DTM(SS)) and anchor (biotin or TCO). Thus, A has only the DTM(SS) cleavable linker and biotin; T has both a DTM(SS) and an Azo linker and TCO; C has both a DTM(SS) and an Azo linker and biotin; and G has only the DTM(SS) cleavable linker and TCO. The four virtual terminators are added together.

After incorporation, an optional replacement of the unlabeled polymerase with polymerase containing the donor dye (Cy3 shown, but Cy2 could also be used) is carried out and background FRET is measured. Next a labeling step is performed using Streptavidin-Cy5 to attach the dye to the biotin anchor on the reversible terminators, and after washing, Cy3-polymerase is added. FRET will be observed for incorporation of either A or C. A second labeling step is performed using Tetrazine-Cy5 to attach the dye to the TCO anchor on the reversible terminators, and after washing, the Cy3-polymerase is added again. The detection of FRET will be indicative of incorporation of T or G. After this, cleavage of the Azo linkers and their attached dyes with sodium dithionite followed by re-addition of Cy3-polymerase and FRET measurement will reveal specifically which nucleotide analogue was incorporated. Loss of signal due to either A or C incorporation will indicate incorporation of C. Loss of signal due to either G or T incorporation will indicate incorporation of T. Remaining signal will indicate the incorporation of A and G, respectively.

Finally, treatment with THP or TCEP will cleave the DTM containing linkers and all blocking groups, removing all remaining dyes, in preparation for the next cycle.

An optional Cy3-polymerase addition will confirm absence of FRET signal. Though not indicated in the scheme, if ensemble sequencing is performed, a chase step can be carried out with non-fluorescent NRTs once during or after each full cycle to guarantee that all the growing primer chains remain in register, in order to avoid skipping bases during the detection steps. In this case cleavage of the 3′ blocking group, e.g., DTM(SS), on these chase nucleotides, is required before commencement of the next cycle.

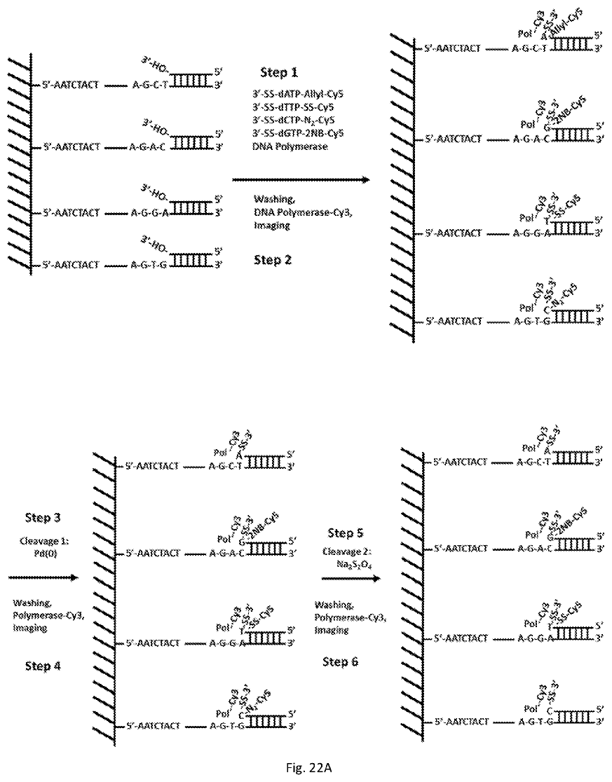

Scheme P6 (FIGS. 21A-21B) is somewhat similar to Scheme P5, again using virtual terminators with a DTM(SS) group in the linker between the base and the blocker, and either a DTM(SS) or Azo group in the linker distal to the blocker to attach to either Cy5 or biotin. Each nucleotide analogue has a different combination of cleavable linker (Azo or DTM(SS)), and dye (Cy5) or anchor (biotin). Thus, A has a DTM(SS) cleavable linker and Cy5, C has an Azo cleavable linker and biotin, G has an SS cleavable linker and biotin, and T has an Azo cleavable linker and Cy5. The four virtual terminators are added together.

After incorporation, an optional replacement of the unlabeled polymerase with polymerase containing the donor dye (Cy3 shown, but Cy2 could also be used) is carried out and FRET is measured. A FRET signal will indicate extension with either A or T. Next a labeling step is performed using Streptavidin-Cy5 to attach the dye to the biotin anchor on the remaining reversible terminators, and after washing, Cy3-polymerase is added. FRET will be observed for incorporation of either C or G. A cleavage step is performed using sodium dithionite to remove the dye from nucleotide analogues with Azo linkers (C and T), and after washing, the Cy3-polymerase is added again. Retention of a FRET signal will be indicative of incorporation of A when the imaging after the extension step indicated A or T; and indicative of G when the imaging after the labeling step indicated C or G. Loss of signal will indicate incorporation of T in the former case and C in the latter case.

Finally, treatment with THP or TCEP will cleave the DTM containing linkers and all blocking groups, removing all remaining dyes, in preparation for the next cycle. An optional Cy3-polymerase addition will confirm absence of FRET signal. In the case of ensemble sequencing, a chase step can be performed exactly as in Scheme P5.

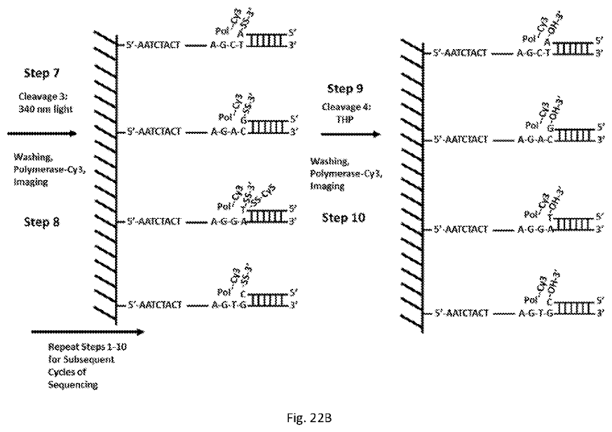

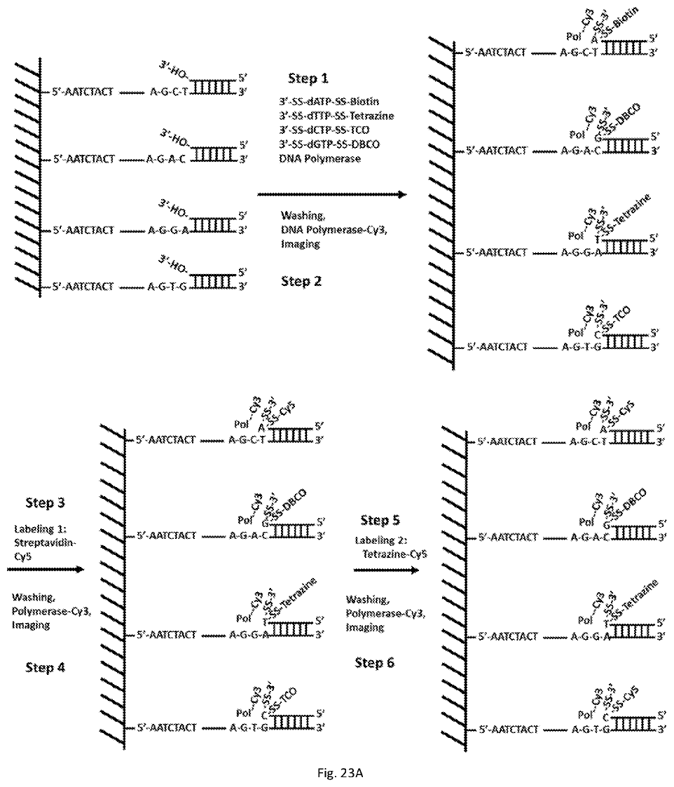

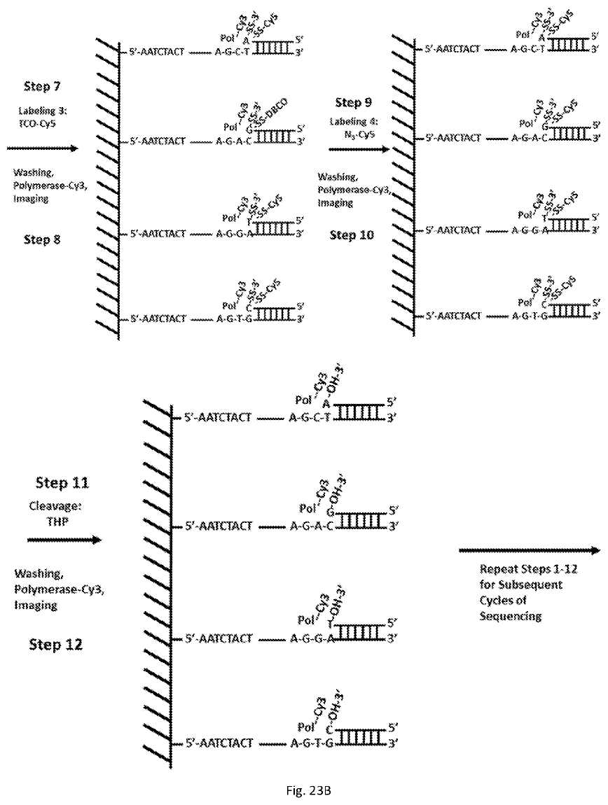

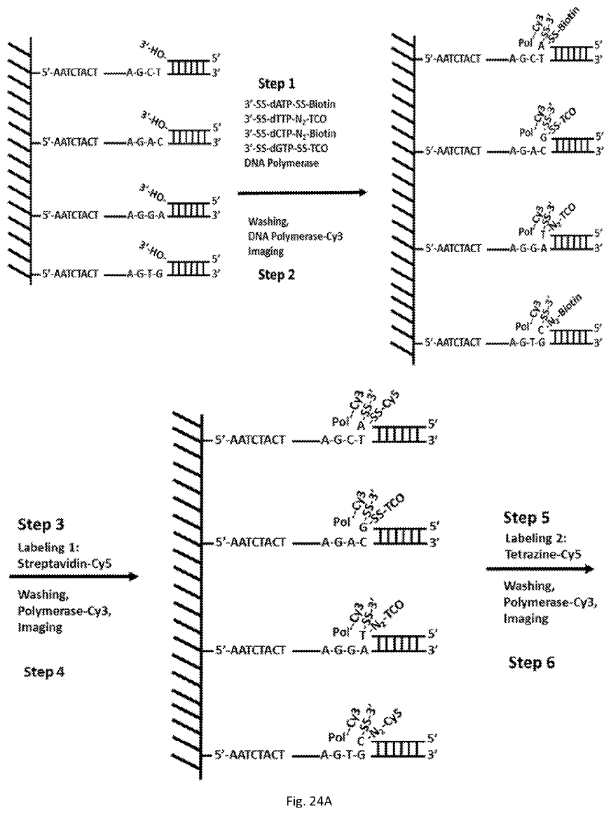

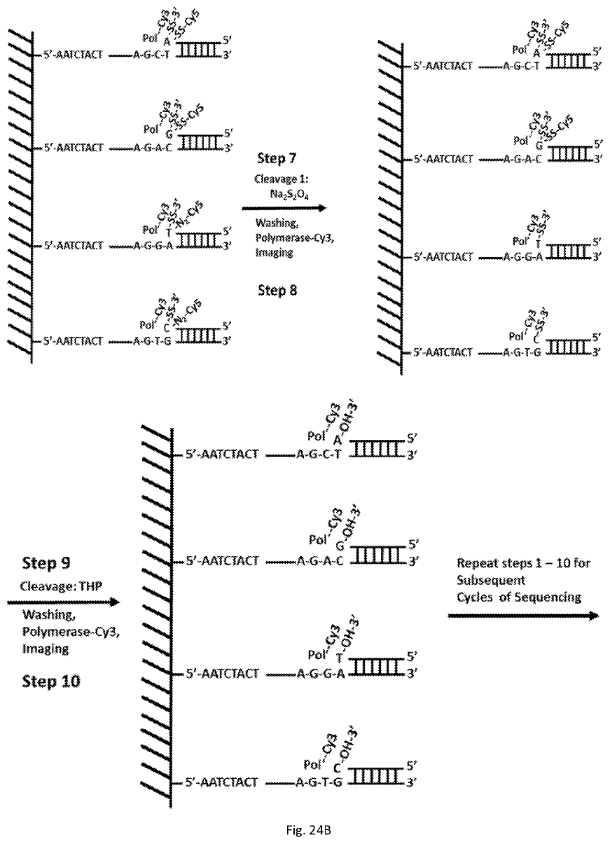

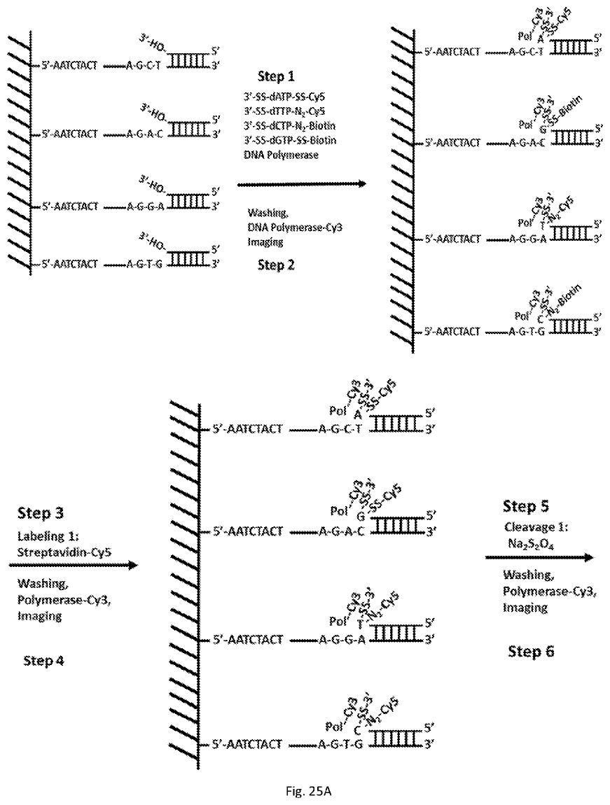

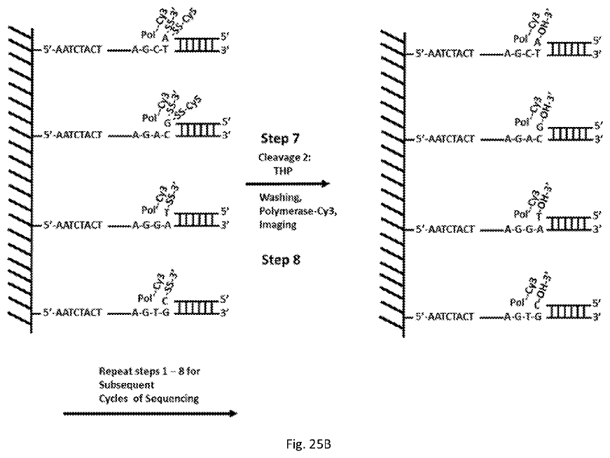

Schemes P7-P10 (FIGS. 22-25) are essentially identical to Schemes P3-P6, respectively, except that nucleotide reversible terminators with 3′-DTM(SS) blockers are used instead of virtual terminators. In Scheme P7 (FIGS. 22A-22B), the cleavable groups on the linkers between the base and the acceptor dye are DTM(SS) for T, allyl for A, Azo for C, and 2-nitrobenzyl for G. In Scheme P8 (FIGS. 23A-23B), the anchors are biotin for A, TCO for C, DBCO for G and tetrazine for T, with Cy5 labeled anchor binding molecules, streptavidin, tetrazine, N₁ and TCO respectively. In Scheme P9 (FIGS. 24A-24B), the orthogonal set of cleavable groups in the linkers and the anchors to which they are attached are DTM(SS) and biotin for A, Azo and TCO for T, Azo and biotin for C, and DTM(SS) and TCO for G. In Scheme P10 (FIGS. 25A-25B), the orthogonal set consists of a DTM(SS) linker between the base and Cy5 for A, an Azo linker between the base and biotin for C, a DTM(SS) linker between the base and biotin for G, and an Azo linker between the base and Cy5 for T. In the final TCEP or THP treatment step, not only are any remaining dyes removed, but the 3′ blocking group is also cleaved restoring the 3′-OH group in readiness for the next cycle of sequencing by synthesis.

Section II: New SBS Methods Involving Energy Transfer with Acceptor Fluorophore on Reversible Terminator and Donor Fluorophore on Adjacent Unincorporable Nucleotide:

The invention described herein provides an approach in which the donor and acceptor dyes are positioned on two adjacent nucleotides where the first (more 5′) incorporable nucleotide is a reversible terminator (virtual terminator or reversibly 3′-blocked dNTP) bearing an acceptor dye (e.g., Cy5) and the second (more 3′) nucleotide is an unincorporable nucleotide bearing a donor dye (e.g., Cy3). (Alternatively, a mixture of Cy5-labeled ddNTPs and unlabeled 3′-blocked dNTPs can be used, along with the Cy3-labeled unincoroporable nucleotides, but only for ensemble sequencing.) A wide variety of unincorporable nucleotides have been described including analogues with α,β-methylene or phosphorothioate instead of natural triphosphate or polyphosphate groups; many other examples are known and several are illustrated in Ju et al, WO 2017/176677 A1, with 4 examples in FIG. 13 herein.

The idea behind this approach is that the unincorporable nucleotides will bind to the position 3′ to the NRT incorporation site, and stay long enough for FRET to occur. While there are many possible schemes, some of which are shown in the examples below, for ease of introducing the approach, the non-limiting examples of the schemes described herein show the general case in which the acceptor dye-containing nucleotides are added one by one (e.g., A, G, C, T, A, G, C, T, etc.). To further simplify these general schemes for ease of understanding, the non-limiting examples described herein are show with traditional nucleotide reversible terminators (NRTs) having a cleavable blocking group at the 3′ O position and a dye attached via a cleavable linker on the base (5 position of pyrimidines or 7 position of purines).

Coincident with or subsequent to carrying out extension in the presence of template, primer, polymerase and one of the acceptor dye-labeled NRTs (e.g., A), a set of the 4 unincorporable nucleotides with attached donor dyes is added, and energy transfer is measured (excitation of donor dye and detection of acceptor dye emission). If the A was incorporated, energy transfer will occur. Cleavage is carried out to remove the acceptor dye and 3′-blocking group from the NRT; there is no need to cleave the dye on the unincorporable nucleotides which are simply washed away. Then the next acceptor dye-labeled NRT (e.g., G) and the set of 4 unincorporable nucleotides with attached donor dyes are added, FRET is measured, and acceptor dyes and blocking groups cleaved. The process is repeated as long as needed to sequence the template.

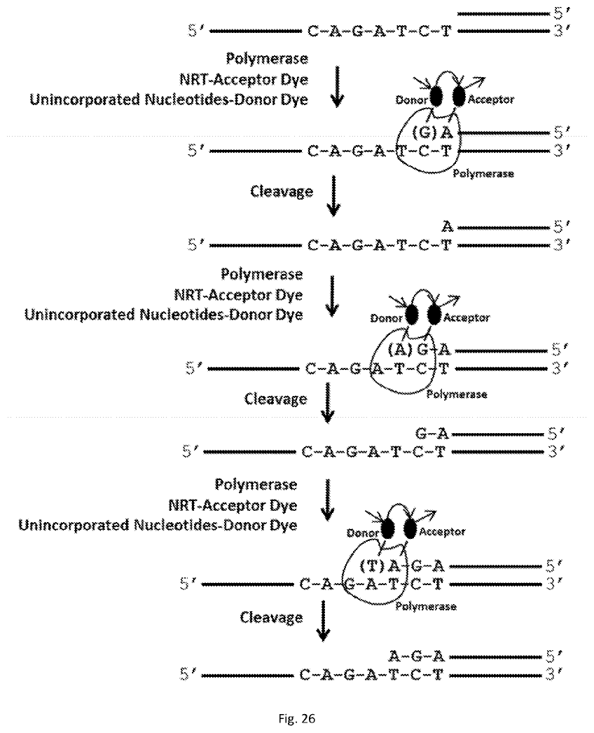

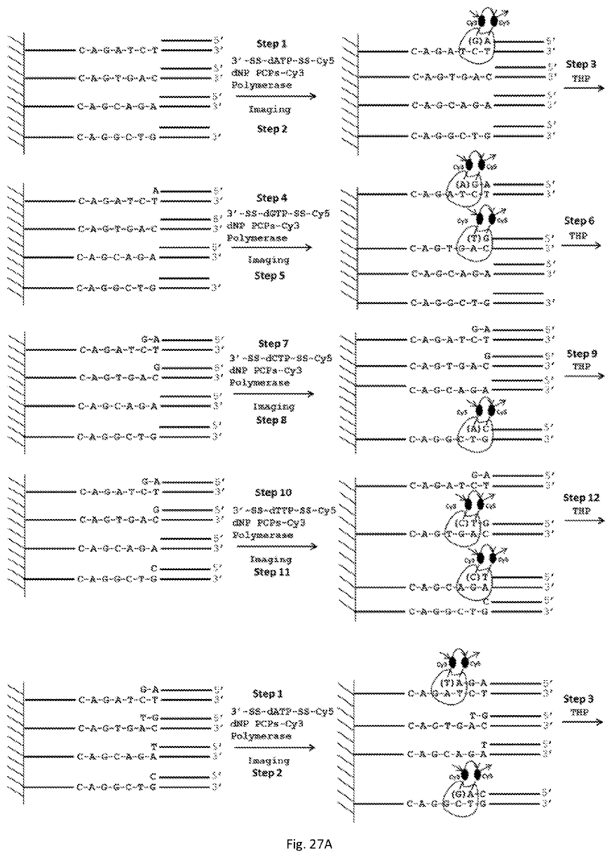

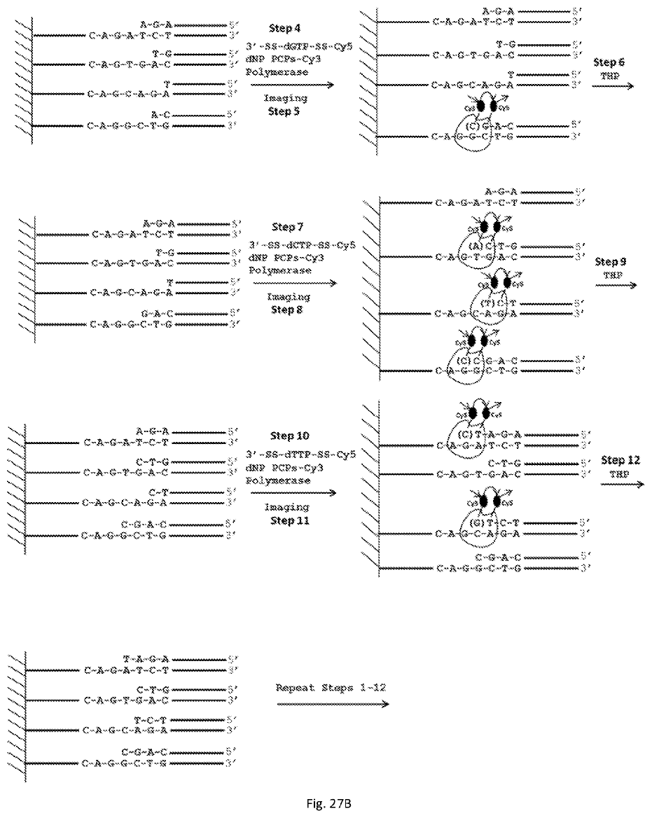

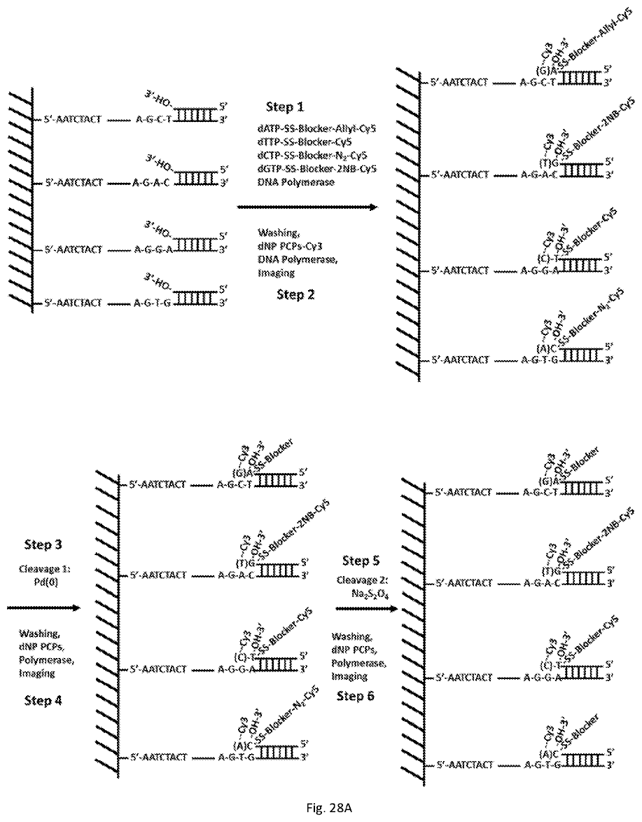

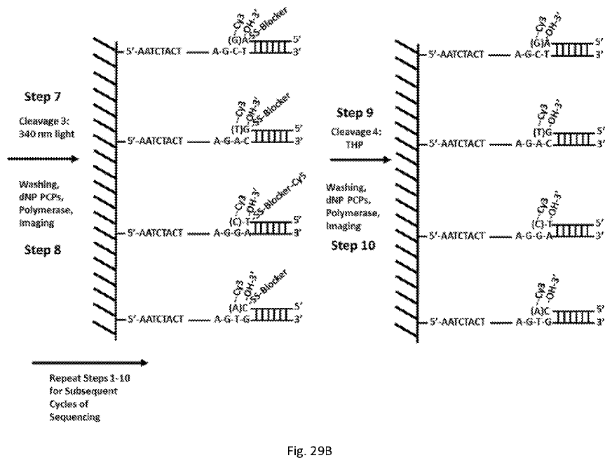

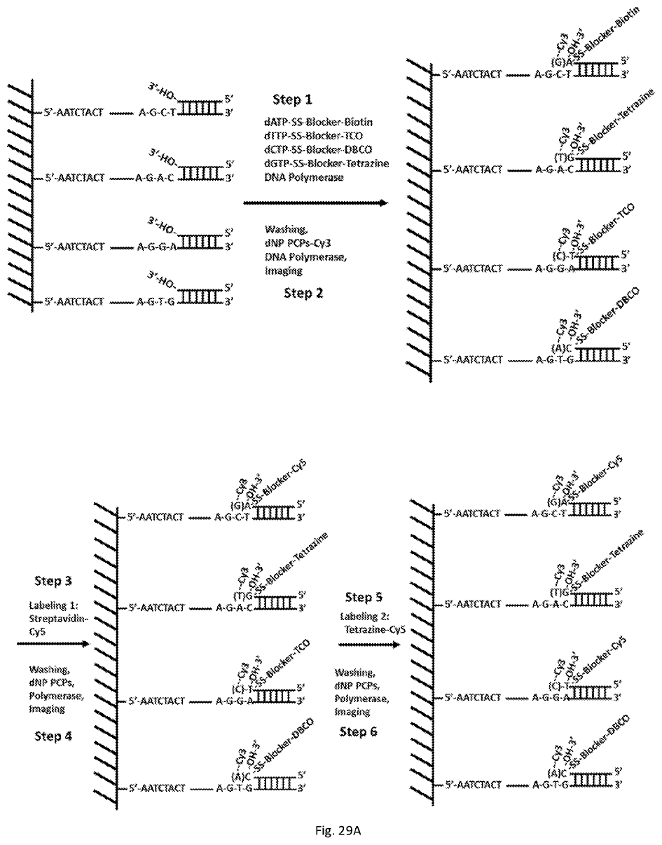

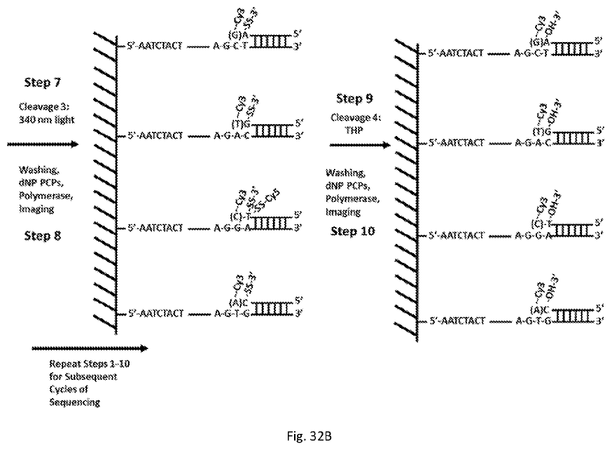

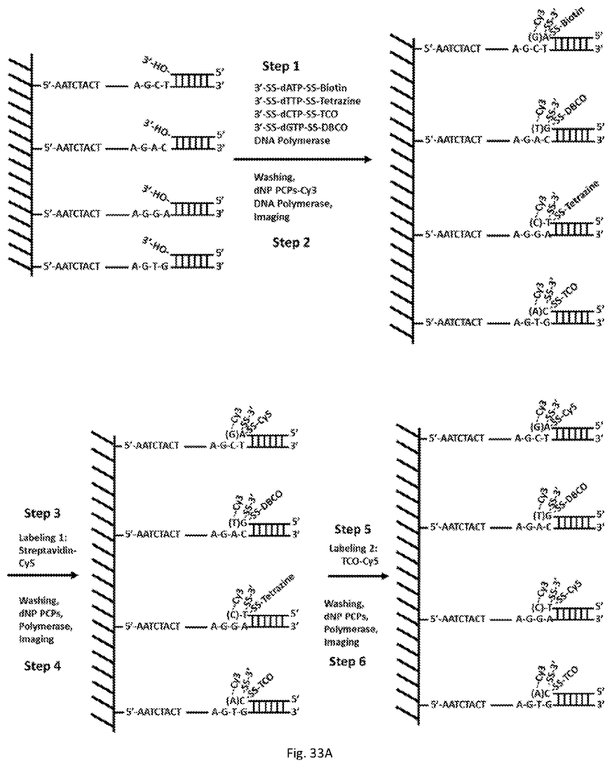

The use of 3′-blocked NRTs (or virtual terminators, if desired) as opposed to natural nucleotides bearing the acceptor dyes is necessary to accurately decode homopolymer stretches (e.g., AAA or GG). (While all four unincorporable nucleotides (A, C, G and T) can be added together, one could simply add a universal unincorporable nucleotide analog (e.g., 2′-deoxyinosine, 2′-deoxynebularine, etc.). The above example requires the four NRTs to be added one by one (Schemes U1 (FIG. 26) and U2 (FIGS. 27A-27B)). Other single-color approaches can also take advantage of the use of donor dye-containing unincorporable nucleotides for energy transfer to an acceptor dye on the NRTs, allowing all four NRTs to be added in each round of SBS. For instance, if a different cleavable linker (azo, allyl, 2-nitrobenzyl, dithiomethyl) is used for attachment of the acceptor dye to the 4 NRTs, FRET can be measured after specifically cleaving each linker (sodium dithionite, Pd(0), ˜340 nm light, TCEP), as in Schemes U3 (FIGS. 28A-28B) and U7 (FIGS. 32A-32B).

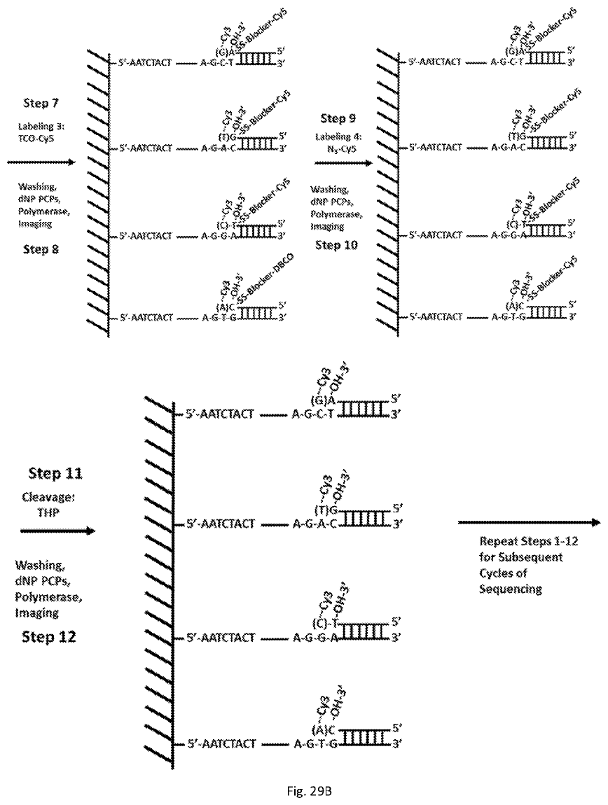

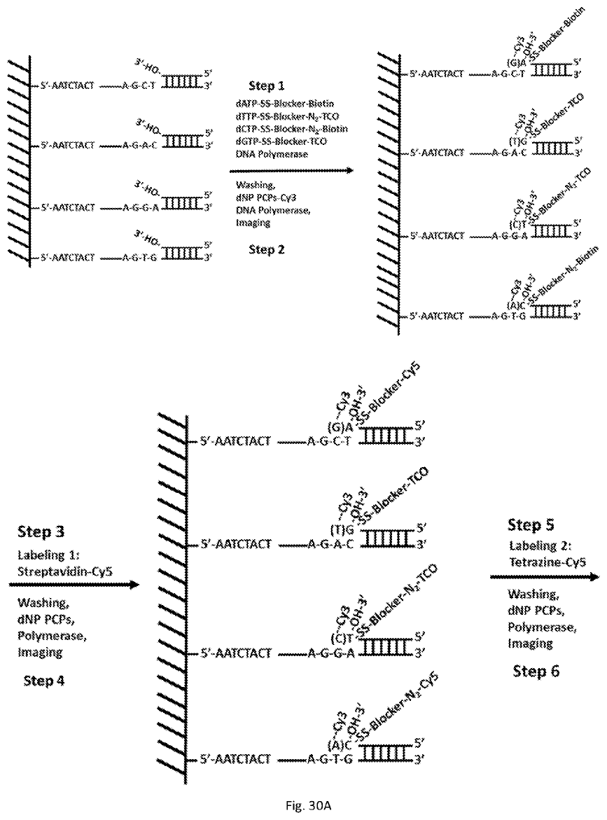

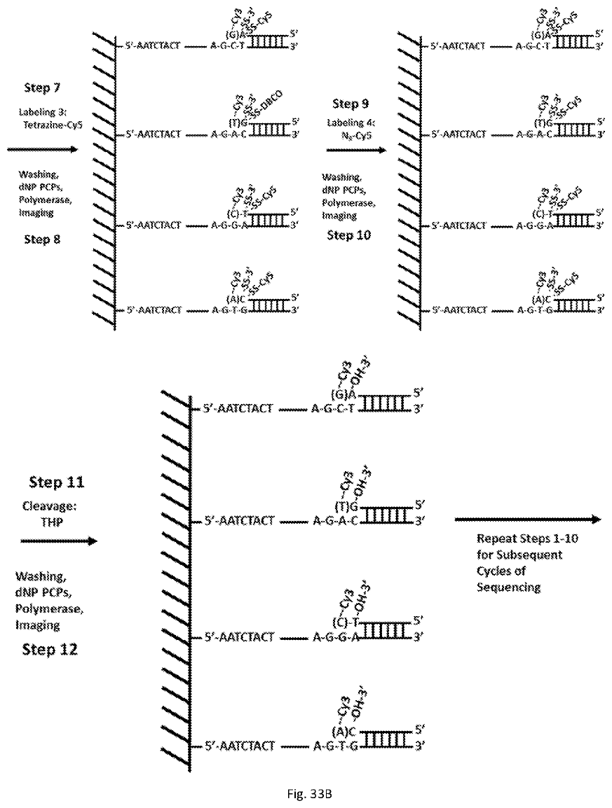

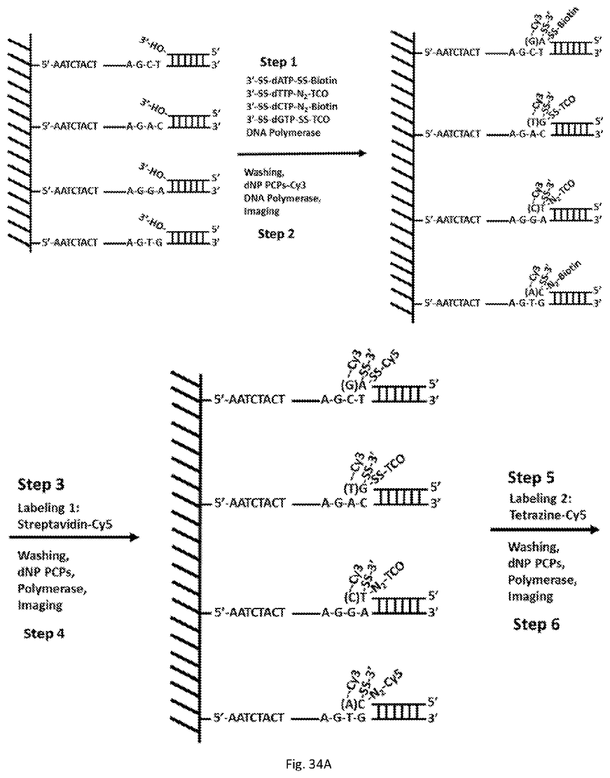

Similarly, if NRTs with one of four different anchors (biotin, TCO, tetrazine, azide) and equivalent acceptor dye-containing anchor binding molecules (streptavidin, tetrazine, TCO, DBCO) are used, FRET can be measured after each labeling reaction, as in Schemes U4 (FIGS. 29A-29B) and U8 (FIGS. 33A-33B).

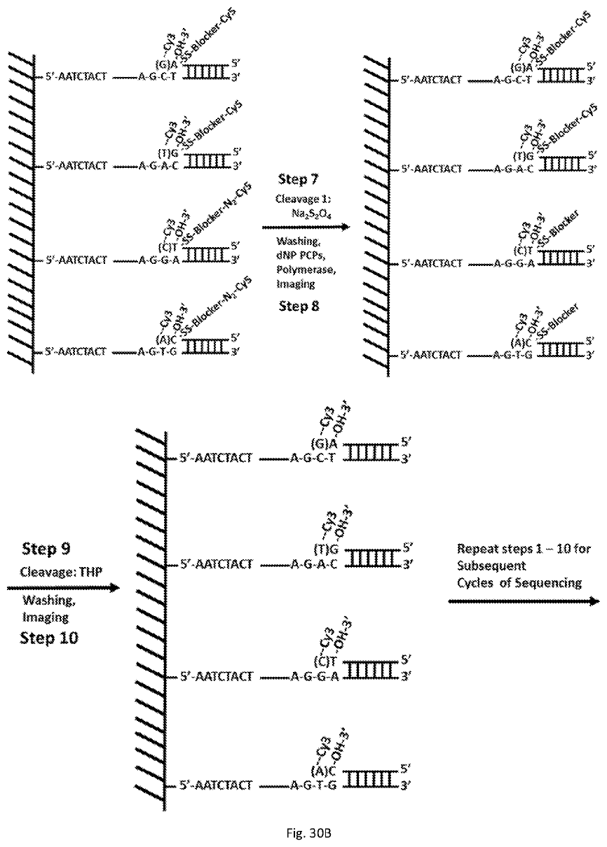

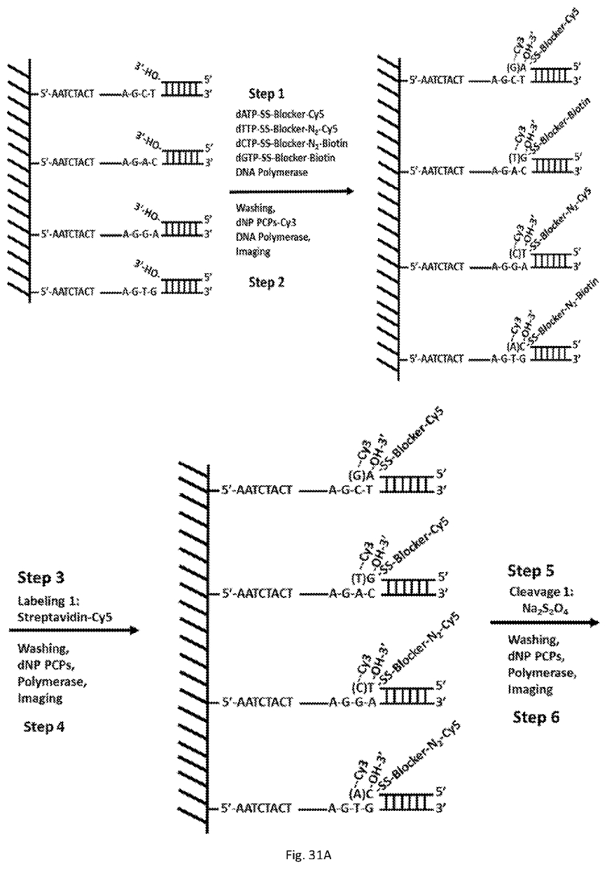

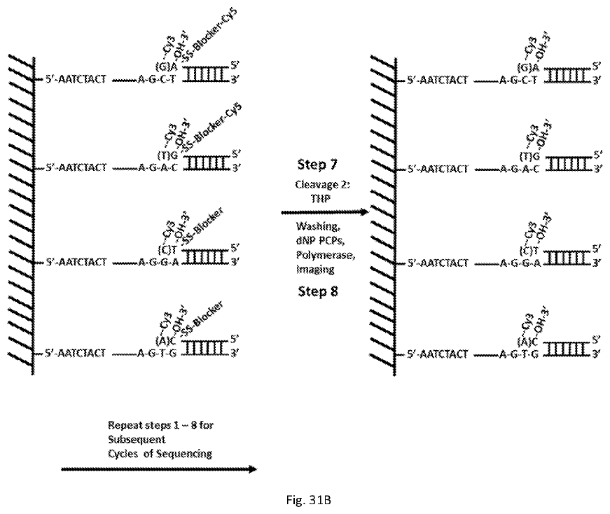

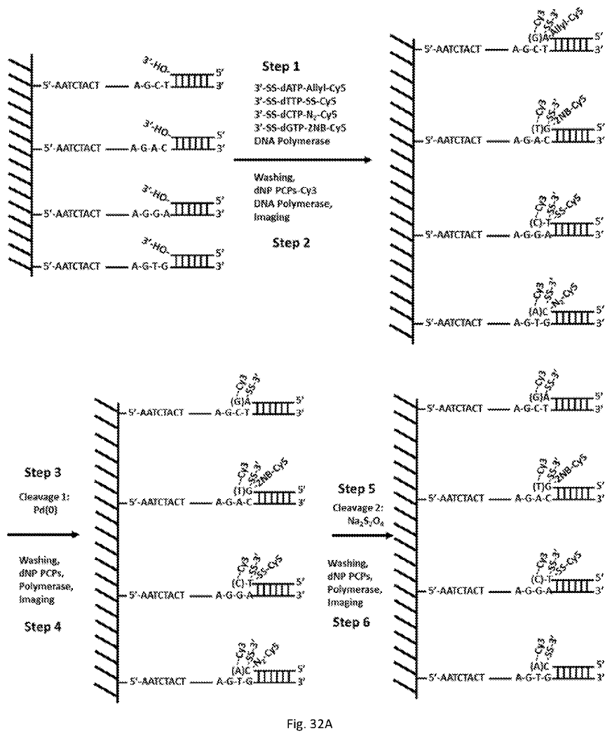

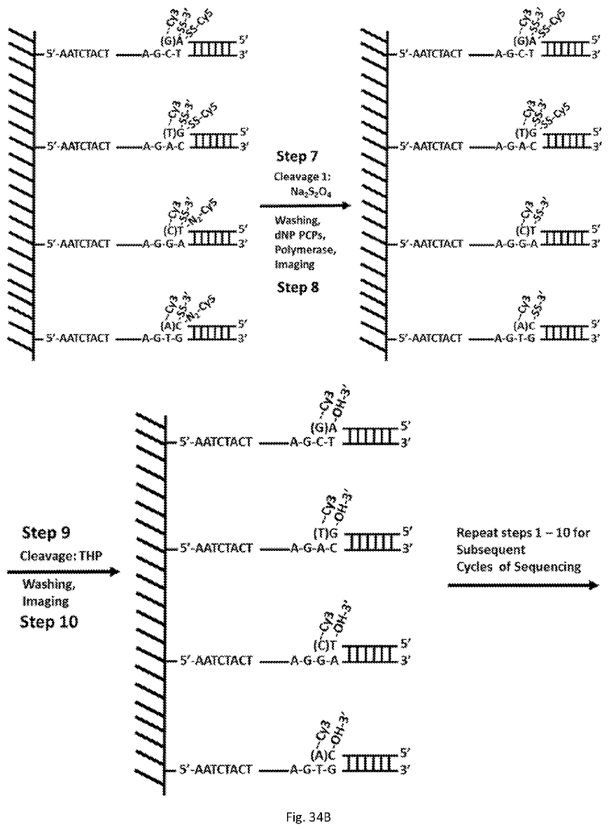

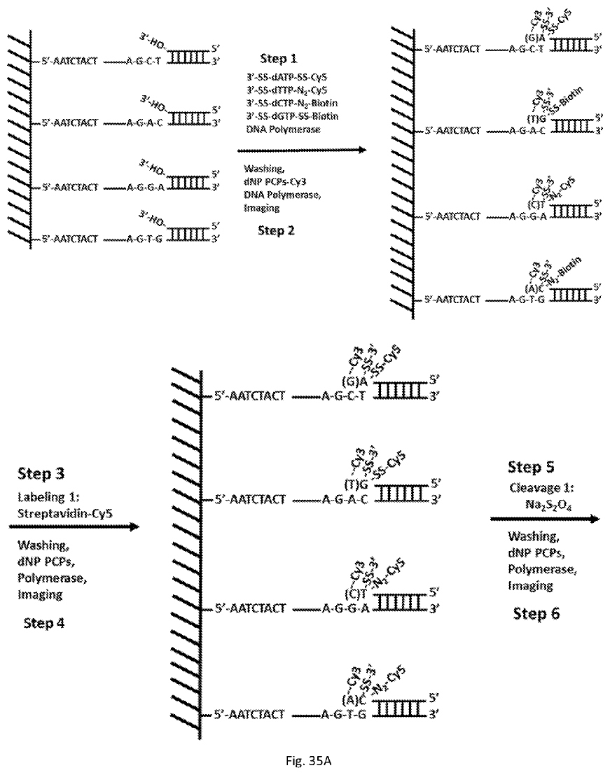

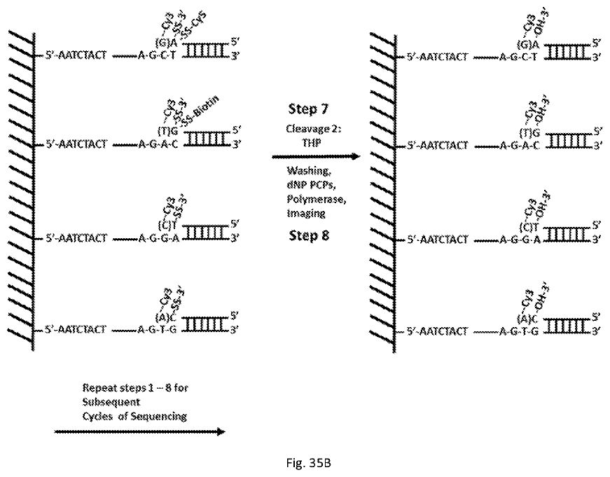

Finally, an orthogonal set of acceptor dye-labeled NRTs possessing either of 2 anchors and either of 2 cleavable linkers can be used, with FRET measured after labeling and cleavage steps (Schemes U5 (FIGS. 30A-30B) and U9 (FIG. 34A-34B), or an orthogonal set with 2 cleavable linkers but with two of these directly linked to the Cy5 and the other two directly attached to biotin, with FRET measured after extension, labeling and cleavage steps (Schemes U6 (FIGS. 31A-31B) and U10 (FIGS. 35A-35B)).

The non-limiting examples of the invention provided herein only show examples with virtual terminators or 3′-blocked NRTs, either of which may be used for both ensemble and single molecule SBS schemes. A hybrid approach incorporating low concentrations of acceptor-labeled dideoxynucleotides in combination with a higher concentration of unlabeled NRTs, along with the unincorporable donor dye-containing unincorporable nucleotides, is also possible and within the scope of the present invention, but only for ensemble sequencing. Finally, while it may be preferable to place the donor dye on the unincorporable nucleotides and the acceptor dye on the NRTs, the opposite placement is also feasible and within the scope of the present invention.

### Example U1: Single Color SBS with Energy Transfer Between Donor Dye on Unincorporable Nucleotide of Growing Primer Strand and Acceptor Dye on Incorporable Nucleotide Analogue with One-by-One Addition of the 4 Incorporable Nucleotide Analogues (Schemes U1 and U2)

The general scheme presented in Scheme U1 demonstrates the basic 1-color method with addition of one nucleotide analogue (3′-blocked NRT or virtual terminator) at a time. The example illustrated in Scheme U1 (FIG. 26) assumes that the correct nucleotide analogue has been added in each cycle of addition. In the initial step, the DNA polymerase reaction permits incorporation, at the 3′ end of the growing primer chain, of the nucleotide analogue complementary to the base at the same position on the template DNA. This nucleotide has a FRET acceptor fluorophore attached via a cleavable linker. Simultaneously or subsequently, a set of unincorporable nucleotides labeled with a FRET donor fluorophore is added. These will bind intermittently and transiently but not be incorporated at the position immediately 3′ of the incorporated reversible terminator. (Although all four unincorporable nucleotides can be added, a single (or perhaps two types of) unincorporable nucleotides with a universal base that can bind with A, C, T and G can be used as well.) The donor and acceptor dyes are brought close enough to each other for energy transfer to take place from donor to acceptor when the donor is excited near its absorption maximum.

The use of rigid or flexible linkers of varying lengths can be used to maximize the FRET. The resulting emission of the acceptor fluorophore (and if desired, the emission of the donor fluorophore) is measured. These excitation, energy transfer, and emission events are indicated by the arrows in the scheme exemplified in FIG. 26. In the next step, the acceptor dyes and any groups that block further nucleotide incorporation are cleaved. These steps are repeated in each cycle. Washes are carried out between steps to remove unincorporated NRTs, polymerases, chemical cleavage agents and other reaction components.

Scheme U2 (FIGS. 27A-27B) provides a specific example of Scheme U1, in which multiple cycles are carried out, using Cy3 as donor and Cy5 as acceptor, and with addition of NRTs in the order A, G, C, T, A, G, C, T, etc. As with all such methods involving addition of one nucleotide at a time, the progression of SBS will be sequence dependent. Thus, in Scheme U2, the 4 DNA templates are extended at somewhat different rates, at least over the short distances shown. Though attachment of the donor dye molecule(s) to the unincorporable nucleotides and acceptor dyes to the NRTs is the method shown in the examples, the opposite placement is also feasible. The use of 3′-blocked NRTs (or virtual terminators, if desired) instead of natural nucleotides is necessary to accurately decode homopolymer stretches (e.g., AAA or GG).

### Example U2: Single Color SBS with Energy Transfer Between Donor Dye on Unincorporable Nucleotide of Growing Primer Strand and Acceptor Dye on Incorporable Nucleotide Analogue with Simultaneous Addition of the 4 Incorporable Nucleotide Analogues (Schemes U3-U10)

In Scheme U3 (FIGS. 28A-28B), the approach is shown with virtual terminators (nucleotide analogues bearing bulky groups (blockers) attached to the base via a cleavable linker) that largely prevent incorporation of the subsequent nucleotide). In addition to this blocking group, the FRET acceptor fluorescent dye (Cy5) is also attached via a second linker, distal to the blocker, on the base. In the example scheme depicted, the linkers for the 4 different bases have a DTM(SS) group between the base and the blocking group, and 3 different cleavable groups, Azo for C, allyl for A, 2-nitrobenzyl for G, or no additional cleavable group between the blocking group and the dye for T. Structures of these and other cleavable linkers are presented in FIG. 3B.

The four virtual terminators are added together. Subsequently (or simultaneously), the four donor dye (Cy3, but Cy2 could also be used)-containing unincorporable nucleotides are added. FRET is measured, presenting as a non-specific signal due to incorporation of any of the 4 nucleotides. In the following steps, the linkers on the reversible nucleotides are cleaved one by one, and addition of the unincorporable nucleotides along with polymerase and imaging is repeated to determine whether the FRET signal is retained or lost. Loss of the FRET signal immediately reveals the specific nucleotide analogue incorporated.

Thus loss of signal after cleavage of the allyl linker with Pd(0) indicates incorporation of A, loss of signal after cleavage of the Azo linker with sodium dithionite indicates incorporation of C, loss of signal after photocleavage of the 2-NB linker with ˜340 nm light indicates incorporation of G, and loss of signal after cleavage of the DTM(SS) linker with THP (which should always be the final cleavage reaction in each cycle) indicates incorporation of T. The THP cleavage also removes all the blocking groups, readying the extended primer for the next cycle of SBS.

Though not indicated in the scheme exemplified in FIGS. 28A-28B, if ensemble sequencing is performed, a chase step can be carried out with non-fluorescent NRTs once during or after each full cycle to guarantee that all the growing primer chains remain in register, in order to avoid skipping bases during the detection steps. In this case cleavage of the 3′ blocking group, e.g., DTM(SS) or azidomethyl, on these chase nucleotides, is required before commencement of the next cycle.

In Scheme U4 (FIGS. 29A-29B), again for virtual terminators, instead of sequential cleavage of the dyes on the four bases in each cycle, the approach involves sequential labeling of the four bases. The blocking groups on the virtual terminators are all connected to the base via the same cleavable linker, and a non-cleavable linker to one of four different anchors is present, distal to the blocker.

In the example scheme depicted in FIGS. 29A-29B, the linkers for the 4 different bases have a DTM(SS) group between the base and the blocking group, and the anchors for A, C, G and T are biotin, DBCO, Tetrazine and TCO respectively. The four virtual terminators are added together. Structures of these and other anchors and anchor binding molecules are presented in FIG. 3B.

After incorporation and addition of polymerase and unincorporable nucleotides containing the donor dye (Cy3 shown, but Cy2 could also be used), FRET is measured, presenting as a non-specific signal due to incorporation of any of the 4 nucleotides. In the following steps, dyes attached to anchor-binding molecules are added one by one, and imaging is repeated in the presence of polymerase and unincorporable nucleotides labeled with donor dye to see if a new FRET signal appears. Gain of the FRET signal immediately reveals the specific nucleotide analogue incorporated. Thus, gain of FRET signal after labeling with streptavidin-Cy5 indicates extension with A, gain of FRET signal after labeling with N₃-Cy5 indicates extension with C, gain of FRET signal after labeling with TCO-Cy5 indicates extension with G, and gain of FRET signal after labeling with Tetrazine-Cy5 indicates extension with T. Finally, treatment with THP removes all the blocking groups, readying the extended primer for the next cycle of SBS. With ensemble sequencing, a chase step can be carried out in the same way as described in Scheme U3 (FIGS. 28A-28B).

In Scheme U5 (FIGS. 30A-30B), the approach is shown with virtual terminators as in Scheme U3 (FIGS. 28A-28B), in this case with a DTM(SS) group between the base and the blocker, and either a DTM(SS) or Azo linker to the anchor molecule (biotin or TCO), distal to the blocking group. Each nucleotide analogue has a different combination of cleavable linker (Azo or DTM(SS)) and anchor (biotin or TCO). Thus, A has only the DTM(SS) cleavable linker and biotin; T has both a DTM(SS) and an Azo linker and TCO; C has both a DTM(SS) and an Azo linker and biotin; and G has only the DTM(SS) cleavable linker and TCO. The four virtual terminators are added together.

Subsequently or simultaneously, the set of unincorporable nucleotides with the attached donor dye (Cy3 shown, but Cy2 could also be used) is added and background FRET is measured. Next a labeling step is performed using Streptavidin-Cy5 to attach the dye to the biotin anchors on the reversible terminators, and after washing, the set of Cy3-labeled unincorporable nucleotides is added. FRET will be observed for incorporation of either A or C. A second labeling step is performed using Tetrazine-Cy5 to attach the dye to the TCO anchors on the reversible terminators and after washing, the set of Cy3-labeled unincorporable nucleotides is added again. The detection of FRET will be indicative of incorporation of T or G. After this, cleavage of the Azo linkers and their attached dyes with sodium dithionite followed by re-addition of Cy3-labeled unincorporable nucleotides and FRET measurement will reveal specifically which nucleotide analogue was incorporated.

Loss of signal due to either A or C incorporation will indicate incorporation of C. Loss of signal due to either G or T incorporation will indicate incorporation of T. Remaining signal will indicate the incorporation of A and G, respectively. Finally, treatment with THP or TCEP will cleave the DTM containing linkers and all blocking groups, removing all remaining dyes, in preparation for the next cycle.

An optional Cy3-polymerase addition will confirm absence of FRET signal. Though not indicated in the scheme, if ensemble sequencing is performed, a chase step can be carried out with non-fluorescent NRTs once during or after each full cycle to guarantee that all the growing primer chains remain in register, in order to avoid skipping bases during the detection steps. In this case cleavage of the 3′ blocking group, e.g., DTM(SS), on these chase nucleotides, is required before commencement of the next cycle.

Scheme U6 (FIGS. 31A-31B) is somewhat similar to Scheme U5 (FIGS. 30A-30B), again using virtual terminators with a DTM(SS) group in the linker between the base and the blocker, and either a DTM(SS) or Azo group in the linker distal to the blocker to attach to either Cy5 or biotin. Each nucleotide analogue has a different combination of cleavable linker (Azo or DTM(SS)), and dye (Cy5) or anchor (biotin). Thus, A has a DTM(SS) cleavable linker and Cy5, C has an Azo cleavable linker and biotin, G has an SS cleavable linker and biotin, and T has an Azo cleavable linker and Cy5. The four virtual terminators are added together. After incorporation, an optional addition of polymerase along with unincorporable nucleotides containing the donor dye (Cy3 shown, but Cy2 could also be used) is carried out and FRET is measured. A FRET signal will indicate extension with either A or T. Next a labeling step is performed using Streptavidin-Cy5 to attach the dye to the biotin anchor on the remaining reversible terminators, and after washing, polymerase and unincorporable nucleotides containing the donor dye are added. FRET will be observed for incorporation of either C or G. A cleavage step is performed using sodium dithionite to remove the dye from nucleotide analogues with Azo linkers (C and T), and after washing, the polymerase and donor dye-labeled unincorporable nucleotides are added again. Retention of a FRET signal will be indicative of incorporation of A when the imaging after the extension step indicated A or T; and indicative of G when the imaging after the labeling step indicated C or G. Loss of signal will indicate incorporation of T in the former case and C in the latter case. Finally, treatment with THP or TCEP will cleave the DTM containing linkers and all blocking groups, removing all remaining dyes, in preparation for the next cycle. An optional imaging step after addition of polymerase and unincorporable nucleotides with donor dye will confirm absence of FRET signal. In the case of ensemble sequencing, a chase step can be performed exactly as in Scheme P5 (FIGS. 20A-20B).

Exemplified Schemes U7 (FIGS. 32A-33B), U8 (FIGS. 33A-33B), U9 (FIGS. 34A-34B) and U10 (FIGS. 35A-35B) are essentially identical to Schemes U3 (FIGS. 28A-28B), U4 (FIGS. 29A-29B), U5 (FIGS. 30A-30B) and U6 (FIGS. 31A-31B) respectively, except that nucleotide reversible terminators with 3′-DTM(SS) blockers are used instead of virtual terminators. In Scheme U7 (FIGS. 32A-33B), the cleavable groups on the linkers between the base and the acceptor dye are DTM(SS) for T, allyl for A, Azo for C, and 2-nitrobenzyl for G. In Scheme U8 (FIGS. 33A-33B), the anchors are biotin for A, TCO for C, DBCO for G and tetrazine for T, with Cy5 labeled anchor binding molecules, streptavidin, tetrazine, N₃ and TCO respectively. In Scheme U9 (FIGS. 34A-34B), the orthogonal set of cleavable groups in the linkers and the anchors to which they are attached are DTM(SS) and biotin for A, Azo and TCO for T, Azo and biotin for C, and DTM(SS) and TCO for G. In Scheme U10 (FIGS. 35A-35B), the orthogonal set consists of a DTM(SS) linker between the base and Cy5 for A, an Azo linker between the base and biotin for C, a DTM(SS) linker between the base and biotin for G, and an Azo linker between the base and Cy5 for T. In the final TCEP or THP treatment step, not only are any remaining dyes removed, but the 3′ blocking group is also cleaved restoring the 3′-OH group in readiness for the next cycle of sequencing by synthesis.

The same FRET acceptor dye-labeled nucleotide reversible terminators and virtual terminators can be used for the schemes exemplified in Sections I and II. Examples of these are presented in FIGS. 4-12, with potential cleavable groups for linkers between base and dye or anchor illustrated in FIGS. 3A-3B. The 3′-blocked nucleotide reversible terminators exemplified in FIG. 4 can be used with Schemes P1 (FIG. 16), P2 (FIGS. 17A-17B), U1 (FIG. 26), and U2 (FIGS. 27A-27B). The virtual terminators exemplified in FIG. 5 can be used with Schemes P3 (FIGS. 18A-18B) and U3 (FIGS. 28A-28B). The virtual terminators exemplified in FIG. 6 can be used with Schemes P4 (FIGS. 19A-19B) and U4 (FIGS. 29A-29B). The virtual terminators exemplified in FIG. 7 can be used for Schemes P5 (FIGS. 20A-20B) and U5 (FIGS. 29A-29B). The virtual terminators exemplified in FIG. 8 can be used with Schemes P6 (FIGS. 21A-21B) and U6 (FIGS. 30A-30B). The 3′-blocked nucleotide reversible terminators (NRTs) exemplified in FIG. 9 can be used with Schemes P7 (FIGS. 22A-22B) and U7 (FIGS. 31A-31B). The NRTs exemplified in FIG. 10 can be used with Schemes P8 (FIGS. 23A-23B) and U8 (FIGS. 32A-32B). The NRTs exemplified in FIG. 11 can be used with Schemes P9 (FIGS. 24A-24B) and U9 (FIGS. 33A-33B). The NRTs exemplified in FIG. 12 can be used with Schemes P10 (FIGS. 25A-25B) and U10 (FIGS. 34A-34B).

Many other sets of nucleotide analogues with different acceptor dyes or dye clusters, and different combinations of anchors and cleavable groups in linkers, can be used. Schemes for synthesis of similar molecules have been presented in Ju et al. PCT/US2019/022326, which is hereby incorporated by reference in its entirety.

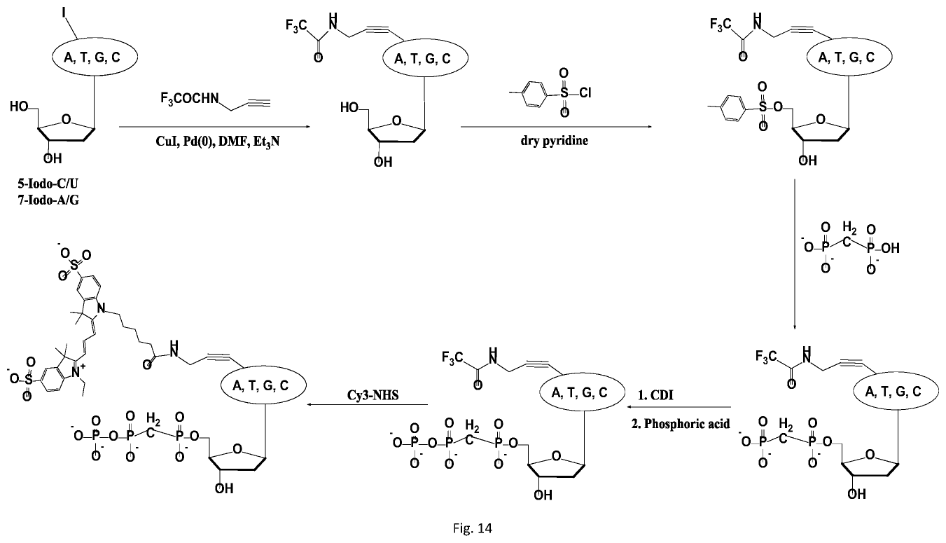

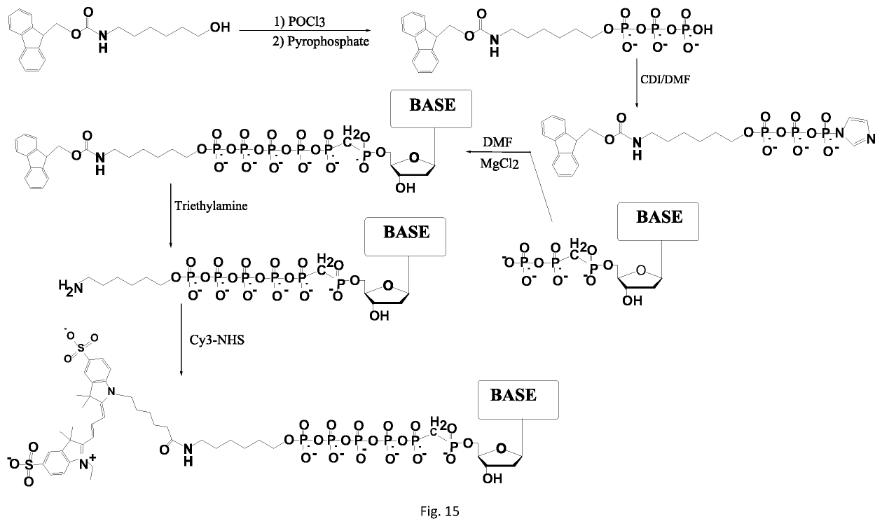

FIG. 13 provides examples of FRET donor-dye labeled unincorporable nucleotides for use with Schemes U1-U10 (FIGS. 26-35), and synthetic schemes for two example unincorporable nucleotides, with dyes attached to the base or terminal phosphate, respectively, are presented in FIGS. 14 and 15. Synthetic schemes for a variety of unincorporable nucleotides have been described in the literature (Liang et al. (2008; Gharizadeh et al. (2002)).

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

### REFERENCES

Bowers, J., Mitchell, J., Beer, E., Buzby, P. R., Causey, M., Efcavitch, J. W., Jarosz, M., Krzymanska-Olejnik, E., Kung, L., Lipson, D., Lowman, G. M., Marappan, S., McInerney, P., Plat, A., Roy, A., Siddiqi, S. M., Steinmann, K., Thompson, J. F. (2009) Virtual terminator nucleotides for next-generation DNA sequencing. Nature Meth 6:593-595.
Braslavsky I, Hebert B, Kartalcv E, Quake S R (2003) Sequence information can be obtained from single DNA molecules. Proc Natl Acad Sci USA 100:3960-3964.
Duthie R S, Kalve I M, Samcls S B, Hamilton S, Livshin I, Khot M, Nampali S, Kumar S, Fuller C W (2002) Novel cyanine dye-labeled dideoxynucleoside criphosphates for DNA sequencing. Bioconj Chem 13:699-706.
Eriksen M, Horvath P, Sorensen M A, Semsey S, Oddershede L B, Jauffred L (2013) A novel complex: A quantum dot conjugated to an active T7 RNA polymerase. J Nanomaterials:ID 468105, 9 pages.
Gharizadeh B, Nordstrom T, Ahmadian A, Ronaghi M, Nyren P. Long-read pyrosequencing using pure 2′-deoxyadenosine-5‘—O’-(1-thiotriphosphate) Sp-isomer. Anal Biochem 301:82-90.
Fuller C, Kumar S, et al (2016) Real-time single molecule electronic DNA sequencing by synthesis using tagged-nucleotides on Genia chip. Proc Natl Acad Sci USA 113:5233-5238.
Guo J, Xu N, Li Z, Zhang S, Wu J, Ki, D H, Marma M S, Meng Q, Cao H, Li X, Shi S, Yu L, Kalachikov S, Russo J J, Turro N J, Ju J (2008) Four-color DNA sequencing with 3′-O-modified nucleotide reversible terminators and chemically cleavable fluorescent dideoxynucleotides. Proc Natl Acad Sci USA. 105: 914-950.
Hobbs F W Jr, Trainor G L (1992) U.S. Pat. No. 5,131,507 A. Alkynylamino-nucleotides. Hung S C, Ju J, Mathies R A, Glazer A N (1996) Cyanine dyes with high absorption cross section as donor chromophores in energy transfer primers. Anal Biochem 243:15-27.
Ju J (2007) DNA sequencing by nanopore using modified nucleotides. U.S. Pat. No. 8,889,348 B2, US 20090298072 A1.
Ju J, Chen X, Li X, Li Z, Hsieh M K, Chien M, Shi S, Ren J, Guo C, Kumar S, Russo J J, Tao C, Jockusch S, Kalachikov S (2017) WO 2017/205336 A1. Nucleotide derivatives and methods of use thereof.
Ju J, Cho Y, Kumar S, Kalachikov S, Tao C, Chien M, Russo J J (2016) WO 2016/154215 A1. Polymer tagged nucleotides for single molecule electronic SNP assay.
Ju J, Glazer A N, Mathies R A (1996) Cassette labeling for facile construction of energy transfer fluorescent primers. Nucleic Acids Res 24:1144-1148.
Ju J, Kim D H, Guo J, Meng Q, Li Z, Cao H (2016) US 2016/0024574 A1. DNA sequencing with non-fluorescent nucleotide reversible terminators and cleavable label modified nucleotide terminators.
Ju J, Kumar S, Li Z, Tac C, Chien M, Russo J J, Kalachikov S, Shepard K, Rosenstein J K (2013) DNA sequencing by synthesis using modified nucleotides and nanopore detection. EP 2652153 A2 (WO 2012083249 A2)
Ju J, Li X, Chen X, Li Z, Kumar S, Shi S, Guo C, Ren J, Hsieh M K, Chien M, Tao C, Erturk E, Kalachikov S, Russo J J (2017) WO 2017/058953 A1. Design and synthesis of novel disulfide linker based nucleotides as reversible terminators for DNA sequencing by synthesis.
Ju J, Wu J, Li Z (2017) U.S. Pat. No. 9,624,539. DNA sequencing by synthesis using Raman and Infrared spectroscopy detection.
Ju J, Kumar S, Russo J, Jockusch S, Li Z, Li X, Kalachikov S, Morozova I (2017 WO 2017/176677 A1. Fluorescence Energy Transfer-Based Single Molecule/Ensemble DNA Sequencing by Synthesis.
Ju Ju, Li X, Kumar S, Chen X, Russo J, Chien M, Jockusch S, Tao C, Wang X, Kalachikov S, Morozova I, Shi S (2019) PCT/US2019/022326. Nucleotide Analogues and Use Thereof for Nucleic Acid Sequencing and Analysis.
Kumar S, Sood A (2006) Labeled Nucleoside Polyphosphates. U.S. Pat. No. 7,041,812 Kumar S, Tao C, Chien M, Hellner B, Balijepalli A, Robertson J W F, Li Z, Russo J J, Reiner J E, Kasianowicz J., Ju J (2012) PEG-Labeled Nucleotides and Nanopore Detection for Single Molecule DNA Sequencing by Synthesis. Scientific Reports 2, 684.
Liang F, Jain N, Hutchens T, Shock D D, Beard W A, Wilson S H, Chiarelli M P, Cho B P (2008) Alpha,beta-methylene-2′-deoxynucleoside 5′-triphosphates as noncleavable substrates for DNA polymerases: isolation, characterization, and stability studies of novel 2′-deoxycyclonucleosides, 3,5′-cyclo-dG, and 2,5′-cyclo-dT. J Med Chem 51:6460-70.
Mitra R D, Shendure J, Olejnik J, Edyta Krzymanska O, Church G M (2003) Fluorescent in Situ sequencing on polymerase colonies. Anal Biochem 320:55-65.
Seo, T S, Bai X, Kim D H, Meng Q, Shi S, Ruparel H, Li Z, Turro N J, Ju J (2005) Four-color DNA sequencing by synthesis on a chip using photocleavable fluorescent nucleotides. Proc Natl Acad Sci USA 102:5926-5931.
Turcatti G, Romieu A, Fedurco M, Tairi A P (2008) A new class of reversible fluorescent nucleotides: synthesis and optimization as reversible terminators for DNA sequencing by synthesis. Nucleic Acids Res 36:e25.
Yang Y Y, Grammel M, Raghavan A S, Charron G, Hang H C (2010) Comparative analysis of cleavable azobenzene-based affinity tags for biorthogonal chemical proteomics. Chem Biol 17:1212-1222.

1. A method of sequencing nucleic acid comprising:
a) providing at least one nucleic acid template hybridized to a primer;
b) providing a first nucleic acid polymerase and extending the primer hybridized to said at least one nucleic acid template with the first nucleic acid polymerase and a fluorescently labeled nucleotide analogue if the nucleotide analogue is complementary to a nucleotide residue which is immediately 5′ to the nucleotide residue of the nucleic acid template hybridized to the 3′ terminal nucleotide residue of the primer, wherein the nucleotide analogue is either:
(i) a fluorescently labeled nucleotide analogue comprising a base and a fluorescent label and a blocking group attached to the base of the nucleotide analogue via a cleavable linker, wherein said blocking group prevents or greatly reduces incorporation of a subsequent nucleotide analogue into the extended primer strand, and wherein said fluorescent label comprises an energy transfer acceptor or donor dye; or
(ii) a fluorescently labeled reversibly blocked nucleotide analogue comprising a base and a fluorescent label attached to the nucleotide analogue via a cleavable linker and a blocking group at the 3′-OH position, wherein said blocking group prevents incorporation of a subsequent nucleotide analogue into the extended primer strand, and wherein the fluorescent label comprises an energy transfer acceptor or donor dye;
c) removing the first nucleic acid polymerase and providing a second nucleic acid polymerase having an attached fluorescent label, wherein said fluorescent label comprises an energy transfer acceptor or donor dye for the energy transfer acceptor or donor dye attached to the nucleotide analogue in step b;
d) identifying the fluorescence signal due to incorporation of the fluorescently labeled nucleotide analogue onto the primer;
e) optionally extending any unextended primer with a 3′ blocked nucleotide analogue without any base modifications;
f) cleaving the label and the blocking group from any incorporated nucleotide analogue of step b);
g) wherein if no fluorescence signal is detected in step d), iteratively repeating steps b) to f) with a fluorescently labeled nucleotide analogue having a different base until the fluorescently labeled nucleotide analogue is incorporated;
h) optionally extending any unextended primer with a 3′ blocked nucleotide analogue without any base modifications if the optional chase step e) was not carried out;
i) iteratively performing steps b) to h) for each nucleotide residue of the nucleic acid template,
thereby determining the sequence of the nucleic acid template.

2. The method of claim 1, wherein the fluorescently labeled nucleotide analogue has a blocking group attached to the base or at the 3′-OH position.

3. The method of claim 1, wherein the dye on the nucleotide analogue is Cy5 or ATTO647N and the dye on the second nucleic acid polymerase of step c) is Cy3, and/or wherein the cleavable linker on the base is DTM.

4. The method of claim 1, wherein the 3′ blocking group of the fluorescently labeled nucleotide analogue is DTM or azidomethyl, and the cleavage is carried out with THP.

5. The method of claim 1, wherein the dye on the fluorescently labeled nucleotide analogues is an energy transfer donor dye and the dye on the second nucleotide polymerase is an energy transfer acceptor dye, or wherein the dye on the fluorescently labeled nucleotide analogues is an energy transfer acceptor dye and the dye on the second nucleotide polymerase is an energy transfer donor dye, and/or wherein the dye on the nucleotide analogue and/or second nucleic acid polymerase is a dye cluster.

6. The method of claim 1, wherein the fluorescently labeled nucleotide analogue is selected from any one of the nucleotide analogues of FIG. 4, FIG. 5, and/or FIG. 9.

7. (canceled)

8. A kit comprising all the required nucleotide analogues, polymerases, cleavage agents and other reaction buffer components for carrying out the method of claim 1.

9. A method of sequencing nucleic acid comprising:
a) providing at least one nucleic acid template hybridized to a primer;
b) providing a first nucleic acid polymerase and four different labeled nucleotide analogues (A, C, T, G) and extending the primer hybridized to said at least one nucleic acid template with the first nucleic acid polymerase and one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
(i) fluorescently labeled nucleotide analogues comprising a base and a blocking group linked to the base via a cleavable linker and a fluorescent label linked distal to the blocking group via either an uncleavable or a different cleavable linker, wherein said blocking group prevents or greatly reduces incorporation of a subsequent nucleotide analogue into the extended primer strand, wherein each of the different nucleotide analogues (A, C, G, T) have the same fluorescent label and different cleavable linkers, and wherein said fluorescent label comprises an energy transfer acceptor or donor dye; or
(ii) fluorescently labeled nucleotide analogues comprising a base and a fluorescent label attached to the base via a cleavable linker and a blocking group at the 3′-OH position, wherein said blocking group prevents incorporation of a subsequent nucleotide analogue into the extended primer strand, and wherein each of the different nucleotide analogues (A, C, G, T) have the same fluorescent label and different cleavable linkers, and wherein said fluorescent label comprises an energy transfer acceptor or donor dye;
c) removing the first nucleic acid polymerase and providing a second nucleic acid polymerase having an attached fluorescent label, wherein said fluorescent label comprises an energy transfer acceptor or donor dye for the energy transfer acceptor or donor dye attached to the nucleotide analogue incorporated in step b;
d) optionally extending any unextended primer with a 3′ blocked nucleotide analogue without any base modifications;
e) identifying the fluorescence resonance energy transfer (FRET) signal due to incorporation of the fluorescently labeled nucleotide analogue;
f) contacting the incorporated labeled nucleotide analogues with a cleaving agent that cleaves the cleavable linker to remove the label from one of the four different labeled nucleotide analogues, wherein said cleaving agent does not cleave the cleavable label from the remaining labeled nucleotide analogues;
g) replenishing the second nucleic acid polymerase and identifying any loss of FRET signal due to the cleavage carried out in step f) to partially or completely identify the incorporated nucleotide;
h) iteratively repeating steps f) and g) with a cleaving agent that cleaves the cleavable linker to remove the label from a different labeled nucleotide analogue, wherein said cleaving agent does not cleave the label from the remaining labeled nucleotide analogues;
i) determining the labeled nucleotide analogue incorporated in step b) by comparing the results obtained in the multiple iterations of step g); and
j) cleaving the blocking group and at the same time cleaving any remaining fluorescent labels from the extended primers, and iteratively carrying out steps b to j to obtain the sequence of the nucleic acid template.

10. The method of claim 8, wherein the fluorescently labeled nucleotide analogue has a blocking group attached to the base or at the 3′-OH position.

11. The method of claim 8, wherein the dye on the nucleotide analogue is Cy5 or ATTO647N and the dye on the second nucleic acid polymerase of step c) is Cy3, and/or wherein dyes comprise dye clusters.

12. The method of claim 8, wherein the four labeled nucleotide analogues of step b) (i) each comprise the same type of cleavable linker linking the blocking group to the base, and three of the four labeled nucleotide analogues comprise a different cleavable linker linking the fluorescent label distal to the blocking group, and/or wherein the cleavable linkers comprise DTM, azo, allyl and 2-nitrobenzyl and the cleaving agents comprise THP, sodium dithionite, Pd(0) and UV light (˜340 nm) respectively.

13. The method of claim 8, wherein the four labeled nucleotide analogues of step b) (ii), each comprise a different cleavable linker linking the fluorescent label to the base, and wherein the cleaving agent the cleaves one of the cleavable linkers also cleaves the blocking group at the 3′-OH position, and wherein the cleaving agent that cleaves the blocking group at the 3′-OH contacts the incorporated labeled nucleotide analogue in the final iteration of step f), and/or wherein the cleavable linkers comprise DTM, azo, allyl and 2-nitrobenzyl and the cleaving agents comprise THP, sodium dithionite, Pd(0) and UV light (˜340 nm) respectively.

14. The method of claim 8, wherein the dye on the fluorescently labeled nucleotide analogues is an energy transfer donor dye and the dye on the second nucleotide polymerase is an energy transfer acceptor dye, or wherein the dye on the fluorescently labeled nucleotide analogues is an energy transfer acceptor dye and the dye on the second nucleotide polymerase is an energy transfer donor dye, and/or wherein the four labeled nucleotide analogues consist of those found in a) FIG. 5 or b) FIG. 9.

15. A kit comprising all the required nucleotide analogues, polymerases, cleavage agents and other reaction buffer components for carrying out the method of claim 8.

16. A method of sequencing nucleic acid comprising:
a) providing at least one nucleic acid template hybridized to a primer;
b) providing a first nucleic acid polymerase and four different anchor labeled nucleotide analogues (A, C, T, G) and extending the primer hybridized to said at least one nucleic acid template with the first nucleic acid polymerase and one of the anchor labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different anchor labeled nucleotide analogues are either:
(i) anchor labeled nucleotide analogues each comprising a base, a blocking group linked to the base via a cleavable linker, and an anchor linked to the base via an uncleavable linker distal to the blocking group, wherein said blocking group prevents or greatly reduces incorporation of a subsequent nucleotide analogue into the extended primer strand, wherein each different anchor labeled nucleotide analogue (A, C, G, T) has a different anchor and the same cleavable linker; or
(ii) anchor labeled nucleotide analogues each comprising a base, an anchor linked to the base via a cleavable linker, and a blocking group at the 3′-OH position, wherein said blocking group prevents incorporation of a subsequent nucleotide analogue into the extended primer strand, and wherein each different anchor labeled nucleotide analogue (A, C, G, T) has different anchor from the remaining anchor labeled nucleotide analogues and the same linker;
c) removing the first nucleic acid polymerase and providing a second nucleic acid polymerase having an attached fluorescent label, wherein said fluorescent label attached to the polymerase is an energy transfer donor or acceptor dye;
d) optionally extending any unextended primer with a 3′ blocked nucleotide analogue without any base modifications;
e) identifying any background fluorescence resonance energy transfer (FRET) signal;
f) labeling any primer extension products with a fluorescently labeled anchor binding molecule specific for one of the four anchors of the nucleotide analogues of step b), wherein the anchor binding molecule comprises a fluorescent label, wherein said fluorescent label is an energy transfer donor or acceptor dye for the energy transfer donor or acceptor dye attached to the second nucleic acid polymerase;
g) optionally replenishing the second nucleic acid polymerase and identifying any fluorescence resonance energy transfer (FRET) signal due to the anchor binding molecule binding to the anchor labeled nucleotide analogue incorporated in step b);
h) iteratively repeating steps f) and g) with a fluorescently labeled anchor binding molecule specific for each of the remaining anchor labeled nucleotide analogues one by one, wherein the same fluorescent dye is attached to all four anchor binding molecules;
i) determining the specific nucleotide analogue incorporated by comparing the results obtained in the multiple iterations of step a);
j) contacting the incorporated with a cleaving agent to cleave the blocking group and the anchor and fluorescent labels from the incorporated nucleotide analogue of step b); and
iteratively carrying out steps b) to j) to thereby obtain the sequence of the nucleic acid template.

17. The method of claim 15, wherein the four anchor labeled nucleotide analogues are those from a) step b) i) or b) step b) ii), and/or wherein the dyes each comprise dye clusters.

18. The method of claim 15, wherein the four anchor labeled nucleotide analogues of step b) (i) each comprise a blocking group attached to the base via the same cleavable linker, a different anchor attached distal to the blocking group via an uncleavable linker, and wherein the anchor of each anchor labeled nucleotide analogue binds to a different anchor binding molecule, and/or wherein each of the four different anchors independently comprises one of biotin, TCO, DBCO or tetrazine and each of the fluorescently labeled anchor binding molecules independently comprises one of streptavidin, tetrazine, azido and TCO respectively.

19. The method of claim 15, wherein the four anchor labeled nucleotide analogues of step b) ii) each comprise a different anchor attached to the base via the same cleavable linker, and wherein the anchor of each anchor labeled nucleotide analogue binds to a different anchor binding molecule, and/or wherein each of the four different anchors independently comprises one of biotin, TCO, DBCO and tetrazine, and wherein each of the fluorescently labeled anchor binding molecules independently comprises one of streptavidin, tetrazine, azido and TCO respectively.

20. The method of claim 15, wherein the dye on the fluorescently labeled anchor binding molecules is an energy transfer donor dye and the dye on the second nucleotide polymerase is an energy transfer acceptor dye, or wherein the dye on the fluorescently labeled anchor binding molecules is an energy transfer acceptor dye and the dye on the second nucleotide polymerase is an energy transfer donor dye, and/or wherein the anchor labeled nucleotide analogues and corresponding anchor binding molecules are those of a) FIG. 6 or b) FIG. 10.

21. A kit comprising all the required nucleotide analogues, polymerases, labeled anchor binding molecules, cleavage agents and other reaction buffer components for carrying out the method of claim 15

22-61. (canceled)
